SEQUENCE LISTING (99 sequences)

SEQ ID NO: 1 - protein, 180 aa, Phocaeicola vulgatus
MNETELVERCGKGDNLARKQLYERYAGQLMAVCVRYTGDREVAQDVLHDGFLNIFRSFSQFTYKGEGSLKAWLTRIMVNEALGYLRKKASTNQEVIVEELPDVIDGDEDDFEQIPQSVLMQFIKELPDGYRTVFNLYVLEEKGHKEIAEMLGITEHTSSSQLYRAKTLLMKKINDYRKRI

SEQ ID NO: 2 - protein, 431 aa, Phocaeicola vulgatus
MKEKDKWIKNFRTRMEGYSEPAPADLWEQLEKELDTVPKVIPMWRRWQAVAAVALLVVVSSLTVWFWQSPSANYLEKQSAELNVMHESDELAPGSITPEQPMALVVPAARSGKKQHVKVGGVAKAEALAAEQEVLLNKEVEEIQIEENYIEEQKEESVIVEQKQQKQAGRSSYSAAKTNYAYVAPHRKKDRNWTVGLSTGNGTFSSSTSMDGYLPLPNGTRNATMNSAYGVETRAEIDKLVQFNNLSEGKDAQSDIKYRMPVTFGASLRFDLSDDWAVETGVTYTQLSSETRSGTEKNNYGWEEKLHYVGIPLKVNRNIWSNKRFEVYASAGGAVEKCVSGKRSIIGSVSTSNAGKDEQFSGGEENVKVKPLQWSLSAAAGAQFKITEKLGIYAEPGVVYYFDDGSNVNTIRKEHPFNFNIQLGVRFTLPK

SEQ ID NO: 3 - DNA, 1296 bp, Phocaeicola vulgatus
  source 1..1296: mol_type = unassigned DNA
atgaaagaaaaagataaatggataaaaaatttccgcacccggatggagggctattcggaaccggctcctgctgatctttgggaacagttggaaaaggaactggatacggttccgaaagtaattccgatgtggcgtcgctggcaggctgtagcagctgttgccttgttagtggttgtgtcttcattgaccgtatggttttggcagtcaccttctgcaaactatttggagaaacagtcggcagagttgaatgtaatgcacgaatccgatgagttggcgccgggatcaatcactcctgagcaacctatggccttggtagttcctgctgcccgctctgggaaaaaacaacatgtaaaagttggtggggttgctaaggcagaggcattggctgccgaacaggaggttttgctaaataaagaggtggaagagatacaaatagaggaaaattatattgaagagcagaaagaggaatctgttatagtggaacaaaaacagcagaaacaggccggcagatcttcctattcggctgccaaaacgaattacgcttacgtggctcctcatagaaagaaggacagaaactggacggtaggactttctaccggtaatggaaccttttcatcttcgactagtatggatggatatttgcctttgcctaatggcacaaggaatgcaacaatgaatagcgcttatggagtggaaacaagggctgaaattgataagctggttcagttcaataacttgtcggaaggaaaggatgcgcagtctgatataaaatatcgtatgcctgttacttttggagcttcccttcgttttgatttaagtgatgattgggctgtagaaaccggggtgacttatacccagctttcatccgaaacacgttcgggaaccgaaaaaaacaattatggttgggaagagaaactgcattatgtgggtattcctctgaaagtgaaccggaatatatggagtaacaagcggtttgaagtgtatgcctcggcaggtggagcggtagaaaaatgtgtgtctggaaagcggagcattatcggcagtgtttctacatcaaatgcaggaaaagatgaacagttttcgggaggggaggaaaatgtcaaagtgaaacctttgcaatggtctttgtctgcagcggcaggtgcgcagtttaagattacggagaaattgggtatttatgcggagccgggagttgtttattactttgatgacggcagtaatgtaaatacgattcgcaaggaacatccttttaattttaatatacagttgggtgtgcggtttactttgccgaaataa

SEQ ID NO: 4 - protein, 421 aa, Bacteroides salyersiae
MKDEKMKDREMWMDKLKEKLENYSEPIPPFGWEQLEKELVPPVKKRILYPYRRWGAAAAAVLLVVASSLSIYFLNTPTADEIRHTVAPVLASDPDLLPPAHDPDMQVAKVEPVQPRPVIAQARKVTERQGIEPSVPAETVTCPVEEGVNPIREAEAKETLPDETKETKEESGTVVRPHQRSGQDKLHIPAEKPKAKKGRWSVGAAVGNGGGTSFSGNGAVEPLHSSFSDQRLNLAPTAGNEIIQIPNNQVVVFKEGVPYLKRTDEIVDIKHHQPVSFGLSVRKGLAKGFSVETGVTYTLLSSDVQTVVSDARIDQKLHYIGIPVRANWNFFDKDRFTLYVAAGGMVEKCVYGKLGNDKLTVNPLQFSVSGAVGAQFNATKHIGVYVEPGVAYFFDDGSDVQTIRKDTPFNFNLQAGIRFTY

SEQ ID NO: 5 - protein, 441 aa, Bacteroides uniformis
MKERMEDELWLKKIKDKLEDYSEPLPVAGWERLEKELSVSGAPITGPHRMIPFRRWAVAAAAVLLVAVSSVSLWLLQSPVGNEMRHTSVPALAVAPDVLPEQTVPAIRTNSIEPAYRAHGNASAPNKEASHPLVAQHIRISVGEEQQEEMLPVETADEVVGIGQQAEEPVVEDTDHETTMQTEEPETREDRYRPSGRDKLHLPEKNSSGRDAKGWAVGLSVGNTGGFSLANEGEANVMSDYMPGSPIYGGNVDLSSTANGIVTIPDGQELVFKDGMPYLQRREKKIADIDHKQPLSFGVSVRKNLAKGFSVESGLTYTYLASDVRYEGSSEKISQKLHYIGIPVRANWNFVDAKNFTMYVSAGGAIEKCVYGKIGTESETVKPVQLSVMGAVGAQYNISNRVGLYVEPGVSYFFDDGSSIQTIRKENPCNFTLQAGIRLTY

SEQ ID NO: 6 - protein, 414 aa, Parabacteroides merdae
MSNKEMKDKERDIFDDLFRSKLQDFEADTMPGDWEAIADRLPVKKSVPFRRTLRYWGAAAVISLLVVTGGVYVFNRDREHLPIAETIRKETEAVENRLTERSESVTPAVVASKPVDRMAKRAVAQVTAVTTSRSDITVDEVVLSPEEDCDTVVVISSKISEASEAIAVTETATLTGTHFLFADAAPVKTEEKKVRAKRWSFGMGGGSVSAGTSNSLNAYALKNTLMTDQELLQLNSPYFNSQSPKTNIRHKTPVSIGLGVSYSINDRFSLQSGLNYTFLSSEWETGAIYHGETKQKLHFIGIPLSLSYKIAEWKRIQFYAAAGAMTEINVAGKLNAKIFVENNEIRHEKKHIRMKEWMWSVNARAGASYPLLRFLSVYAEVGAGYYFDNGSDIETIRSEKPFNVNLQAGFRFGF

SEQ ID NO: 7 - protein, 452 aa, Prevotella copri
MSNDWTNKLRDQLADYQEPVSHDMWAEIEQSLAQSKKVGDEETDVKKTPQARYVVLKRWSVAAAIALLGVGGSYVFLHDDEPGQANLAVQSSFSTASGTSRTSSAVRAEAQSAGSKNAILLAENNSSAHQVNRHSSGRQLNYSKSSVSFQERTVAADNEVMLAAEVEMPEQSVNAEVNGRELSVLQKQNEDSERQMAVSSNHATKPLEADGGQKLYAGRAQDGHFERSYKDKYEQNWTMNLYAENVSLGSGSDGMSNGMYASSDPLAGGGFADHGVFLAAASPLRYAIPKYVEAKHHAPLAIGAQVGIGLAPRLSLSTGVVYTRVASDFKSYGVSEFDTHQVLHYVGVPLGLNYEVWSTGGFHAYVMAGGEADFNVKNDTKVSGHKEDVKRDGVQFSGKASLGAQYDVTPQVGFYIEPGAKYYFDNGSEIENTFKDKKWNFNLQFGLRIHLK

SEQ ID NO: 8 - protein, 420 aa, Bacteroides caccae
MEEKELWMNKLKEKLEDYSEPIPASGWEQLEKELMPPVERKIYPYRKWMMAAAAVILLALVSSVSLYFLGTPAADEIRHTQTPALASVLDALPAVQQPDAQGATADPVSRPMLKTDRLAKTEHNISEQNTYTDQPVIQNEDEFPATDDKIDNGEKEETKLVKDANAGQKKQALETEEPRNNRPRRPSSRDKYHIPTEKKSSQKGTWSMGFAVGNSGGASTEVGGGTQYLSRVSMAAVSNGLMNIPEDKTLVFEDGIPYLRQAKQVIDIKHHQPISVGVSVRKSLGKGFSVESGLTYTLLSSDVKLADGNQEVEQKLHYVGIPLRANWNFLDKKLVTLYVSGGGMIEKCVYGKLGSEKETVKPLQFSVSGAVGVQLNATKRIGIYMEPGVAYFFDDGSSVQTIRKENPFNFNIQAGIRFTY

SEQ ID NO: 9 - protein, 260 aa, Phocaeicola vulgatus
MRKYKATVIVIIMVMLIPFMQSCLDDYDDDIYDMQAGMPFNAALATVVTPSEIPGEAMIESDNDGVAYVVNPDKLTRFETNNPGQRIFYTYINADNPTGDASKKGPFISIDYLQKILTKRMDTLKENEEDIYGHDGINLITPIMGKTHLTLMFQILGFNSNIKHRISLVATEGTVPDANGYMAVELRHNAEGDRQEYPSSGYVSFPLLDVPGYKEGKLQGFKIKMNTINNGEETVTVSYNKSKSTNFPFIQKGISNTKTK

SEQ ID NO: 10 - protein, 245 aa, Phocaeicola vulgatus
MKKLNLLLLAFLVVMGVTLQSCDDSDGYSLGDVAVDWATVNVKGAHVYDFTGDRWGQIWPATTDCFWYSPIDGQRVILYFNPLYDNYPEGYDCSVKVLNIKEILTKPIEELTAENEEAFGNDPVDIFEGNMWISGGYLNIIFNQNMPSKMKHLVSLVKNTTITPVQDGYIHLEYRYNTYADTTGYWRNGAVSFNLKSLKITSETKGIKVKINSAKNGEKEVSFDLKETPSPVGLSQMDFSQMEIK

SEQ ID NO: 11 - protein, 251 aa, Phocaeicola vulgatus
MKKFFGFNKLLASVAVLASVMIFQSCLDDDDNDYYRYALPNALVTVKPVADNSFFMQLDDSTTLLPVNMKNSPYGEKEVRALVNFDPVNESSGEYDKAVHINWIDSILTKPIAGDLGDKNDEVYGTDPVDMINDWVTIAEDGYLTLRFRTMWGNRSQAHFVNLLMSKDAENPYEVEFRHNAFGDVYGEPADGLVAFNLDSLPDTEGKTVKLKLKWKSFNGEKTAEFDYCTRKSTPANSAAITAGRSVLRLK

SEQ ID NO: 12 - protein, 438 aa, Phocaeicola vulgatus
MRKKILFIIPYIPYPLDSGGNQAFFNMVEYLRHKMAVSVLLYPETGRRMQDVESLKKIWDNVDFFIYTPKTKTVRLPKIKHPFYYKWLQKIHASVTRKMNRQQIWTDETGEVVDLVRGKSTLFSSCFQPLDQGYVEYVSQVAHSGFDIIQVEFYELLSLGFLLPENVQTIFVHHELRYIRNEKEITLFREQTAGDRMLFHIAKDFERSALLKYKDVIVLTEVDRKIMEDFIGRKDHIYTSPAVVQVGDRLEQPFVPVQSGRLTFVGSEDHFPNLDAVAWFCHEVIPHLRKRHFSFTLQVIGKWRGECINRLQSEYPELKLAGYVEDLGSFLKGSVAVVPIRIGSGMRMKILDAVLSKVPFVTTAKGVEGIDFKDGEDCLIVDDPAGFAEAVIALSSNPQLQRQLVTHAEDSLRQVYNPGQMQERRLAVYEQILGDKVG

SEQ ID NO: 13 - protein, 612 aa, Phocaeicola vulgatus
MLKQFFSLLKRYILPYRKYLTWALILNFLSQWLNVFSFMAIVPILNILFKIDTKSYEYIPMDIHNLDKDVLINNAYYFVSNFVATNGAFYTLAMMGGILIFMTMLKTAGYFASAAVMVPLRTGIVRDIRIQVYNKVLSLPLSFFSEERKGDIIARMSADVTVVENSLTSSIDMLIRNPIALLVCFVTLFSVSWQMTLFVIFILPLTGWIMGVVSRKLKRQSSTAQAQWGDIMSQLDETLGGLRVIKAFIAESKMSARFSKTNNDFRDAMNEMIIRQSSAHPMSEFLGTCVIVTVLLFGGALILNTNYAPMDAATFIFYLIILYSIINPLKEFSRAFYNIPQGLASMERIDMILKAENHIVEPEQPLPLDAFTDKLEFKNVSFSYVEGRPVLNHINLTVPKGKTIALVGQSGSGKSTLVDLVPRYHDVSEGALLIDGKNVKDVSIHSLRSLIGNVNQEAILFNDTFYNNITFGVENATMEQVIEAAKIANAHDFIMETEKGYDTMIGDRGGRLSGGQRQRVSIARAILKNPPILILDEATSALDTESERLVQEALERLMKSRTTIAIAHRLSTIKNADEICVLYEGDIVERGTHDELIALNGYYKKLNDMQSL

SEQ ID NO: 14 - protein, 310 aa, Phocaeicola vulgatus
MEQIKKLAIVIPAYKGRFLKETLDSIAVQAHKDEFVLYIGDDASPERLDKIVESYQNKVNLVYHRFSENMGGKDLVAHWERCIQLSAEPFIWLFSDDDLMPADGVERVMEVLSRPHHQRGYFFRFPLAVIDGENKRIRANRPLEEGSVSCYRLLLDKLQGKIDSAAVEYVFSREIWQSAGGFVHFPMAWCSDDATWAAFARHAGGVISLPGQPVCWRNVEGANISNSAGHDKDKLHATILFLRWMRNMFSDYVDDPELISALQCYIHTILRISLHKHYNICGLWGVSMALGRFNKRAAFTTFFRNFRLFS

SEQ ID NO: 15 - protein, 225 aa, Phocaeicola vulgatus
MKILLVEDDANLREVTQRSLEKERYVVEIAADYRTALQKIEDYDYDCILLDIMLPDGSGLDLLEKLKEMHKRENVIILSAKDSIEDKVLGLELGADDYLPKPFHLVELNARIKSVIRRHQQGGENSIKYGNIEIFPDKYGVTVAGKKLELNRKEYDILVYFMNRPGRLVNKSTLAESVWGDHIDQVDNFDFIYAQIKNLRKRLKEAGATPELKAVYGFGYKMVVE

SEQ ID NO: 16 - protein, 428 aa, Phocaeicola vulgatus
MKLIYYIIIRISLVLSVLLTGWAILFYFAVMDEVNDEVDDSLEDYSEIIIIRALAGEELPSKNTASNNQYFLREVTKEYAGSCDDIIYKDSMVYIPEKDETEPARILTTIFKDDGEKFFELTVATPSIEKEDLKDAMAGWIIFLYIALLLTIIVINVWVFYRNMRPLYVLLHWLDKYRIGKVNEPLQNNTRVSEFRKLNEAAVRYAERSEQMFEQQKQFIGNASHEMQTPLAICRNRLEMLMEDENLSESQLEELMKTHQTLEHITKLNKSLLLLSKIENGQFTDTVQVEVNKLLRQYLEDYKEVYQYREIITSIEEEGVFYLTINETLAVVLLTNLLKNAFVHNMDGGHIRIVITPHSVMFCNTGAAQPLDARRIFERFYQGKKKEGSTGLGLAIADTICKMQALRLCYEYKSGEHCFTLYASTHNQ

SEQ ID NO: 17 - DNA, 34 bp, synthetic construct
  source 1..34: mol_type = other DNA
caagggatccataagaaaaaggcacatcgagaac

SEQ ID NO: 18 - DNA, 35 bp, synthetic construct
  source 1..35: mol_type = other DNA
gtcgacgcgtttgttacatgaacacaaaatagctg

SEQ ID NO: 19 - DNA, 34 bp, synthetic construct
  source 1..34: mol_type = other DNA
atgtacgcgttttgttggtaagccagaaattgta

SEQ ID NO: 20 - DNA, 34 bp, synthetic construct
  source 1..34: mol_type = other DNA
cgaaggatccaagatcttctcctatatcgcaagc

SEQ ID NO: 21 - DNA, 34 bp, synthetic construct
  source 1..34: mol_type = other DNA
accaggatccaaacacggtataacaaaagaacca

SEQ ID NO: 22 - DNA, 34 bp, synthetic construct
  source 1..34: mol_type = other DNA
aggaggatccactccttccctattttgtttagga

SEQ ID NO: 23 - DNA, 20 bp, synthetic construct
  source 1..20: mol_type = other DNA
ggtatatactatggagttgc

SEQ ID NO: 24 - DNA, 24 bp, synthetic construct
  source 1..24: mol_type = other DNA
actccttccctattttgtttagga

SEQ ID NO: 25 - DNA, 24 bp, synthetic construct
  source 1..24: mol_type = other DNA
gtggatatttgttttccaacatga

SEQ ID NO: 26 - DNA, 24 bp, synthetic construct
  source 1..24: mol_type = other DNA
aagataaagcgtatggaagcaaag

SEQ ID NO: 27 - DNA, 39 bp, synthetic construct
  source 1..39: mol_type = other DNA
acatcatatgaacaatgaacttcctattgaaaatgtaca

SEQ ID NO: 28 - DNA, 34 bp, synthetic construct; identical to SEQ ID NO: 22
  source 1..34: mol_type = other DNA

SEQ ID NO: 29 - DNA, 37 bp, synthetic construct
  source 1..37: mol_type = other DNA
gataatcctattttaacaaacgcaagttcatcttttg

SEQ ID NO: 30 - DNA, 37 bp, synthetic construct
  source 1..37: mol_type = other DNA
caaaagatgaacttgcgtttgttaaaataggattatc

SEQ ID NO: 31 - DNA, 39 bp, synthetic construct
  source 1..39: mol_type = other DNA
gggactgaaactctcacaaactccgcaagcgatggaggt

SEQ ID NO: 32 - DNA, 39 bp, synthetic construct
  source 1..39: mol_type = other DNA
acctccatcgcttgcggagtttgtgagagtttcagtccc

SEQ ID NO: 33 - DNA, 34 bp, synthetic construct
  source 1..34: mol_type = other DNA
gtgttcatgtaacaatgaacttcctattgaaaat

SEQ ID NO: 34 - DNA, 42 bp, synthetic construct
  source 1..42: mol_type = other DNA
attcgagctcggtacccgggactccttccctattttgtttag

SEQ ID NO: 35 - DNA, 40 bp, synthetic construct
  source 1..40: mol_type = other DNA
aatcagaattgactctagagcacggtataacaaaagaacc

SEQ ID NO: 36 - DNA, 30 bp, synthetic construct
  source 1..30: mol_type = other DNA
gttcattgttacatgaacacaaaatagctg

SEQ ID NO: 37 - DNA, 38 bp, synthetic construct
  source 1..38: mol_type = other DNA
taagattagcattatgagtggcatctaagatagctgac

SEQ ID NO: 38 - DNA, 33 bp, synthetic construct
  source 1..33: mol_type = other DNA
ctttataccagccacatactaataacagaaaac

SEQ ID NO: 39 - DNA, 39 bp, synthetic construct
  source 1..39: mol_type = other DNA
agtatgtggctggtataaagatactaatacacattagag

SEQ ID NO: 40 - DNA, 38 bp, synthetic construct
  source 1..38: mol_type = other DNA
cgaattcctgcagcccgggggctaactcagatgctcgc

SEQ ID NO: 41 - DNA, 39 bp, synthetic construct
  source 1..39: mol_type = other DNA
taagattagcattatgagtgggtaggctcgttgaacaag

SEQ ID NO: 42 - DNA, 33 bp, synthetic construct
  source 1..33: mol_type = other DNA
cagcccatcccataagaaaaggatcactcttcc

SEQ ID NO: 43 - DNA, 30 bp, synthetic construct
  source 1..30: mol_type = other DNA
ttttcttatgggatgggctgaaacggaatc

SEQ ID NO: 44 - DNA, 43 bp, synthetic construct
  source 1..43: mol_type = other DNA
cgaattcctgcagcccggggaagcaatgatggctctttctatc

SEQ ID NO: 45 - DNA, 39 bp, synthetic construct
  source 1..39: mol_type = other DNA
aatcagaattgactctagagtcaagaaaccggaagagtg

SEQ ID NO: 46 - DNA, 38 bp, synthetic construct
  source 1..38: mol_type = other DNA
attcgagctcggtacccgggctggctgcattctccttc

SEQ ID NO: 47 - DNA, 43 bp, synthetic construct
  source 1..43: mol_type = other DNA
cggtgtaagattagcattatgagtgtccgcttcatggagactc

SEQ ID NO: 48 - DNA, 32 bp, synthetic construct
  source 1..32: mol_type = other DNA
agcaatgacacggtgagcttgcacgtacaaac

SEQ ID NO: 49 - DNA, 31 bp, synthetic construct
  source 1..31: mol_type = other DNA
acgtgcaagctcaccgtgtcattgctccttc

SEQ ID NO: 50 - DNA, 43 bp, synthetic construct
  source 1..43: mol_type = other DNA
gatatcgaattcctgcagcccgggggtttcgcggtcactcatc

SEQ ID NO: 51 - DNA, 34 bp, synthetic construct
  source 1..34: mol_type = other DNA
ttctggatcccggatgcagtatttttgcagtctt

SEQ ID NO: 52 - DNA, 31 bp, synthetic construct
  source 1..31: mol_type = other DNA
ctacggatccggttccgaatagccctccatc

SEQ ID NO: 53 - DNA, 34 bp, synthetic construct
  source 1..34: mol_type = other DNA
gatgggatcctacagagccaaaaccttattgatg

SEQ ID NO: 54 - DNA, 40 bp, synthetic construct
  source 1..40: mol_type = other DNA
tcatggatccattttatataagaacagtgaaaaagttcat

SEQ ID NO: 55 - DNA, 34 bp, synthetic construct; identical to SEQ ID NO: 51
  source 1..34: mol_type = other DNA

SEQ ID NO: 56 - DNA, 40 bp, synthetic construct; identical to SEQ ID NO: 54
  source 1..40: mol_type = other DNA

SEQ ID NO: 57 - DNA, 42 bp, synthetic construct
  source 1..42: mol_type = other DNA
agtggatcccccgggctgcaccaaaatagggcacgttatatc

SEQ ID NO: 58 - DNA, 31 bp, synthetic construct
  source 1..31: mol_type = other DNA
tctttatgattacctccagtaccggatattc

SEQ ID NO: 59 - DNA, 37 bp, synthetic construct
  source 1..37: mol_type = other DNA
actggaggtaatcataaagaaatggatggaagtgatc

SEQ ID NO: 60 - DNA, 38 bp, synthetic construct
  source 1..38: mol_type = other DNA
cttgatatcgaattcctgcagtggatgatccggagctg

SEQ ID NO: 61 - DNA, 39 bp, synthetic construct
  source 1..39: mol_type = other DNA
taaacatatgtgtacaaatacttatgacgtttgtgaaat

SEQ ID NO: 62 - DNA, 40 bp, synthetic construct
  source 1..40: mol_type = other DNA
ccaccatatgttatggataaacatatcctaaaataccacc

SEQ ID NO: 63 - DNA, 40 bp, synthetic construct
  source 1..40: mol_type = other DNA
taagattagcattatgagtgcgtaggcaaaaacggcatag

SEQ ID NO: 64 - DNA, 37 bp, synthetic construct
  source 1..37: mol_type = other DNA
ctttgaactacattagtaacttcttacaggtgaatac

SEQ ID NO: 65 - DNA, 28 bp, synthetic construct
  source 1..28: mol_type = other DNA
gttactaatgtagttcaaagtcgggtgg

SEQ ID NO: 66 - DNA, 41 bp, synthetic construct
  source 1..41: mol_type = other DNA
cgaattcctgcagcccggggcgacacggttatttgggatag

SEQ ID NO: 67 - DNA, 28 bp, synthetic construct
  source 1..28: mol_type = other DNA
ttcgtcgattcttgccagttgaacctac

SEQ ID NO: 68 - DNA, 33 bp, synthetic construct
  source 1..33: mol_type = other DNA
aattctgttcccgtattgccttatagaaatttc

SEQ ID NO: 69 - DNA, 28 bp, synthetic construct
  source 1..28: mol_type = other DNA
ggcaatacgggaacagaattggccgaag

SEQ ID NO: 70 - DNA, 30 bp, synthetic construct
  source 1..30: mol_type = other DNA
actgccgagcccaccaattccatattcaag

SEQ ID NO: 71 - DNA, 39 bp, synthetic construct
  source 1..39: mol_type = other DNA
taagattagcattatgagtgggtcgaacttataacgggc

SEQ ID NO: 72 - DNA, 28 bp, synthetic construct
  source 1..28: mol_type = other DNA
aactggcaagaatcgacgaacaccctcc

SEQ ID NO: 73 - DNA, 29 bp, synthetic construct
  source 1..29: mol_type = other DNA
gaattggtgggctcggcagtcgaaagaac

SEQ ID NO: 74 - DNA, 38 bp, synthetic construct
  source 1..38: mol_type = other DNA
cgaattcctgcagcccgggggtgacctcccttgcgctc

SEQ ID NO: 75 - DNA, 36 bp, synthetic construct
  source 1..36: mol_type = other DNA
tcttctaaccgcttgacggaaatcaacaaattttag

SEQ ID NO: 76 - DNA, 34 bp, synthetic construct
  source 1..34: mol_type = other DNA
ttgcactacgcagtttgatttctcaggactttac

SEQ ID NO: 77 - DNA, 38 bp, synthetic construct
  source 1..38: mol_type = other DNA
gctcaacaattgcttgacgggtggagctgatgcgtttc

SEQ ID NO: 78 - DNA, 32 bp, synthetic construct
  source 1..32: mol_type = other DNA
tccgtcaagcggttagaagaattgtggaagtc

SEQ ID NO: 79 - DNA, 28 bp, synthetic construct
  source 1..28: mol_type = other DNA
aatcaaactgcgtagtgcaaatatagcg

SEQ ID NO: 80 - DNA, 40 bp, synthetic construct
  source 1..40: mol_type = other DNA
tgcctgttctatttccgaaccgatgtagccattatcgatg

SEQ ID NO: 81 - DNA, 18 bp, synthetic construct
  source 1..18: mol_type = other DNA
gttcggaaatagaacagg

SEQ ID NO: 82 - DNA, 19 bp, synthetic construct
  source 1..19: mol_type = other DNA
ccgtcaagcaattgttgag

SEQ ID NO: 83 - DNA, 42 bp, synthetic construct
  source 1..42: mol_type = other DNA
aatcagaattgactctagagcgctccttcccttcacttattg

SEQ ID NO: 84 - DNA, 47 bp, synthetic construct
  source 1..47: mol_type = other DNA
attcgagctcggtacccgggcattggtgatgatgctatttaagatag

SEQ ID NO: 85 - DNA, 15 bp, synthetic construct
  source 1..15: mol_type = other DNA
gtacacaccgcccgt

SEQ ID NO: 86 - DNA, 23 bp, synthetic construct
  source 1..23: mol_type = other DNA
ctttctctcttccgtatcattac

SEQ ID NO: 87 - DNA, 19 bp, synthetic construct
  source 1..19: mol_type = other DNA
gctaatcccccaatcatac

SEQ ID NO: 88 - DNA, 21 bp, synthetic construct
  source 1..21: mol_type = other DNA
atcaatattgcgtactcgaac

SEQ ID NO: 89 - DNA, 43 bp, synthetic construct
  source 1..43: mol_type = other DNA
aatcagaattgactctagagctgagttgtaatcaaacctattg

SEQ ID NO: 90 - DNA, 39 bp, synthetic construct
  source 1..39: mol_type = other DNA
attcgagctcggtacccggggatagttaaggtgcatcac

SEQ ID NO: 91 - DNA, 39 bp, synthetic construct
  source 1..39: mol_type = other DNA
aatcagaattgactctagaggtctaactttgcaacgttc

SEQ ID NO: 92 - DNA, 50 bp, synthetic construct
  source 1..50: mol_type = other DNA
attcgagctcggtacccgggttaaaatgaatatccaaaactaatataagg

SEQ ID NO: 93 - DNA, 42 bp, synthetic construct; identical to SEQ ID NO: 83
  source 1..42: mol_type = other DNA

SEQ ID NO: 94 - DNA, 47 bp, synthetic construct; identical to SEQ ID NO: 84
  source 1..47: mol_type = other DNA

SEQ ID NO: 95 - protein, 183 aa, Bacteroides salyersiae
MEEQELTELCRQGDNRARKELYEQYAGRMLGICLRYAGDRETAQDLLHDGFLKIFGSFDKFTWRGEGSLRAWMERVMVNVALQYLRKNDVMSQSMALEDAPETYEEPDASEVEAIPQQVLMHFVEELPAGYRTVFNLYTFEDKSHKEIAQLLGINEKSSASQLFRAKTVLARKVKEWLMMNDR

SEQ ID NO: 96 - protein, 182 aa, Bacteroides uniformis
MEEQELAERCRQGDNLARKELYERYAGRMLSVCLRYAGDRETAQDLMHDGFLKLFDSFDKFTWRGEGSLRAWMERVMVNTVLQYLRKNDVMNQSSALENAPEAYEEPEGSSIDVIPQKVLMQFISELPAGYRTVFNLYIFEEKSHKEIARLLGINEKSSASQLTRAKATLAAKVREWMKRND

SEQ ID NO: 97 - protein, 175 aa, Parabacteroides merdae
MTEKQLIEGCRNGERLAQKELYETYSRKMMGVCLRYVSDRETARDLLQDGFVKVFTSMDSYSGLGSFEGWMRKIFVNCALEYLRKSDVLREAVDLDNTVELVHPDSSAISDMSAVELMKLVQELPAGFRTVFNLFAIEGYSHKEISEMMNITESTSRSQFTRAKQLLQRRIHELY

SEQ ID NO: 98 - protein, 197 aa, Prevotella copri
MKLESEKKLLDDINSGSRAAMHRLYERYVGYAMAVALRYVPMRDDAEDVVQDSFIKVFSGISKFEYRGEGALQAWLLRIVTNEAVNFVRQQKRFTIVDEVPDDIEDEEPEVERVPPAELTRMIGELPDGYRLVLNMFVFEQKSHKEIAQLLGIKESSSASQYLRAKKLLGKKVKDYLSQADCSDIKELNHINEKSNE

SEQ ID NO: 99 - protein, 182 aa, Bacteroides caccae
MEELELSEQCRLGNNQARKELYEQYAGRMLGICLRYTGDRDTAQDLLHDGFIKIFDSFDKFTWRGEGSLRAWMERVIVNTALQYLRKNDVMNQSAPLEELPEKYEEPDVSDVEAIPQRVLMQFIEELPAGYRTVFNLYTFEDKSHKEIAQVLGINEKSSASQLFRAKSVLAKRVKEWIMNNG

(19) United States
(12) Patent Application Publication
(10) Pub. No.: US 2026/0014211 A1
(43) Pub. Date: Jan. 15, 2026

(54) METHODS AND COMPOSITIONS FOR INCREASING BACTERIAL ENGRAFTMENT AND POPULATIONS IN THE GUT MICROBIOME

(71) Applicant: THE UNIVERSITY OF CHICAGO, Chicago, IL (US)

(72) Inventor: Laurie COMSTOCK, Chicago, IL (US)

(21) Appl. No.: 18/995,669

(22) PCT Filed: Jul. 19, 2023
(86) PCT No.: PCT/US2023/070531

§ 371 Date: Jan. 16, 2025

Related U.S. Application Data
(60) Provisional application No. 63/390,424, filed on Jul. 19, 2022.

(51) Int. Cl. A61K 35/74; C07K 14/195; C12N 1/20; C12N 15/74; C12R 1/01

(52) U.S. Cl. CPC A61K 35/74; C07K 14/195; C12N 1/20; C12N 15/74; C12R 2001/01

(57) ABSTRACT

The disclosure relates to methods and compositions that provide for bacterial populations with increased potential for engraftment into the gut of mammalian systems. Accordingly, aspects of the disclosure relate to a bacterial cell or population of cells that comprise at least one of: i) a heterologous nucleic acid encoding a ECF-type sigma factor of the family identified above based on the structure and sequence of cognate anti-sigma factor, or a functional fragment thereof; ii) deletion of a functional ECF-type-associated anti-sigma factor; or iii) a heterologous nucleic acid encoding a ECF-type sigma factor-induced gene and/or an O-antigen inducing gene.

### CROSS-REFERENCE TO RELATED APPLICATIONS

This application claims priority to and the benefit of U.S. Provisional Application No. 63/390,424 filed Jul. 19, 2022, the contents of which are incorporated into the present application by reference in its entirety.

This invention was made with Government support under grant number AI093771 awarded by the National Institutes of Health. The Government has certain rights in the invention.

### SEQUENCE LISTING

The instant application contains a Sequence Listing which has been submitted in XML format and is hereby incorporated by reference in its entirety. Said XML copy, created on Jul. 17, 2023, is named “ARCDP0783WO” and is 95 kilobytes in size.

### BACKGROUND

### I. Field of the Invention

The invention generally relates to the field of medicine. More particularly, it concerns compositions and methods for modifying the gut microbiome.

### II. Background

The human gut microbiota makes critical contributions to host health. However, it has also become increasingly clear that several diseases, drug failures, and other health related properties are associated with an altered but microbiome or the over or under-production of various microbial products. Although cause-and-effect relationships are just beginning to be elucidated in humans, strong associations between gut microbiota composition and microbial products and various pathologies provide a compelling case for the development of strategies that alter the gut microbiota or its products and, ideally, reverse adverse health outcomes or increase health promoting properties and molecules.

One of the strategies used to modulate the gut microbiota is by dietary administration of live microorganisms, often referred to as probiotics or live biotherapeutics. However, the degree to which clinical improvements have been causally linked to probiotic-induced microbiota changes has not been established. Moreover, most probiotic strains do not colonize well at all and must be taken daily. Overall, the engraftment, persistence and ecological impacts of introducing live microbes in the human gut appear limited, and fundamental questions remain regarding how best to establish live microbes in the human gut and the modulation of the microbiome through such an approach. Thus, there is a need in the art for techniques that increase engraftment of bacterial organisms to increase efficacy of probiotics and biotherapeutics.

### SUMMARY OF INVENTION

The disclosure relates to methods and compositions that provide for bacterial populations with increased potential for engraftment into the gut of mammalian systems. Accordingly, aspects of the disclosure relate to a bacterial cell or population of cells that comprise at least one of: i) a heterologous nucleic acid encoding a ECF-type sigma factor of the family identified above based on the structure and sequence of cognate anti-sigma factor, or a functional fragment thereof; ii) deletion of a functional ECF-type-associated anti-sigma factor; or iii) a heterologous nucleic acid encoding a ECF-type sigma factor-induced gene and/or an O-antigen inducing gene. It is specifically contemplated that, in some aspects, the bacterial cell or population of cells do not comprise a heterologous nucleic acid encoding a ECF-type sigma factor of the family identified above based on the structure and sequence of cognate anti-sigma factor, or a functional fragment thereof. It is specifically contemplated that, in some aspects, the bacterial cell or population of cells do not comprise deletion of a functional ECF-type-associated anti-sigma factor. It is specifically contemplated that, in some aspects, the bacterial cell or population of cells do not comprise a heterologous nucleic acid encoding a ECF-type sigma factor-induced gene and/or an O-antigen inducing gene.

The term ECF-type sigma factor refers to a sigma factor that is encoded in the same operon as an antisigma factor having a beta-barrel outer membrane structure at the C-terminus. The antisigma factor genes are much longer than typical anti-sigma factor genes with typical sizes of 1220-1400 bp. Accordingly, the sigma factor may be defined or further defined as one that is encoded in the same operon as endogenous antisigma factor and wherein the endogenous antisigma coding region of the nucleic acid is at least 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, 1000, 1001, 1002, 1003, 1004, 1005, 1006, 1007, 1008, 1009, 1010, 1011, 1012, 1013, 1014, 1015, 1016, 1017, 1018, 1019, 1020, 1021, 1022, 1023, 1024, 1025, 1026, 1027, 1028, 1029, 1030, 1031, 1032, 1033, 1034, 1035, 1036, 1037, 1038, 1039, 1040, 1041, 1042, 1043, 1044, 1045, 1046, 1047, 1048, 1049, 1050, 1051, 1052, 1053, 1054, 1055, 1056, 1057, 1058, 1059, 1060, 1061, 1062, 1063, 1064, 1065, 1066, 1067, 1068, 1069, 1070, 1071, 1072, 1073, 1074, 1075, 1076, 1077, 1078, 1079, 1080, 1081, 1082, 1083, 1084, 1085, 1086, 1087, 1088, 1089, 1090, 1091, 1092, 1093, 1094, 1095, 1096, 1097, 1098, 1099, 1100, 1101, 1102, 1103, 1104, 1105, 1106, 1107, 1108, 1109, 1110, 1111, 1112, 1113, 1114, 1115, 1116, 1117, 1118, 1119, 1120, 1121, 1122, 1123, 1124, 1125, 1126, 1127, 1128, 1129, 1130, 1131, 1132, 1133, 1134, 1135, 1136, 1137, 1138, 1139, 1140, 1141, 1142, 1143, 1144, 1145, 1146, 1147, 1148, 1149, 1150, 1151, 1152, 1153, 1154, 1155, 1156, 1157, 1158, 1159, 1160, 1161, 1162, 1163, 1164, 1165, 1166, 1167, 1168, 1169, 1170, 1171, 1172, 1173, 1174, 1175, 1176, 1177, 1178, 1179, 1180, 1181, 1182, 1183, 1184, 1185, 1186, 1187, 1188, 1189, 1190, 1191, 1192, 1193, 1194, 1195, 1196, 1197, 1198, 1199, 1200, 1201, 1202, 1203, 1204, 1205, 1206, 1207, 1208, 1209, 1210, 1211, 1212, 1213, 1214, 1215, 1216, 1217, 1218, 1219, 1220, 1221, 1222, 1223, 1224, 1225, 1226, 1227, 1228, 1229, 1230, 1231, 1232, 1233, 1234, 1235, 1236, 1237, 1238, 1239, 1240, 1241, 1242, 1243, 1244, 1245, 1246, 1247, 1248, 1249, 1250, 1251, 1252, 1253, 1254, 1255, 1256, 1257, 1258, 1259, 1260, 1261, 1262, 1263, 1264, 1265, 1266, 1267, 1268, 1269, 1270, 1271, 1272, 1273, 1274, 1275, 1276, 1277, 1278, 1279, 1280, 1281, 1282, 1283, 1284, 1285, 1286, 1287, 1288, 1289, 1290, 1291, 1292, 1293, 1294, 1295, 1296, 1297, 1298, 1299, 1300, 1301, 1302, 1303, 1304, 1305, 1306, 1307, 1308, 1309, 1310, 1311, 1312, 1313, 1314, 1315, 1316, 1317, 1318, 1319, 1320, 1321, 1322, 1323, 1324, 1325, 1326, 1327, 1328, 1329, 1330, 1331, 1332, 1333, 1334, 1335, 1336, 1337, 1338, 1339, 1340, 1341, 1342, 1343, 1344, 1345, 1346, 1347, 1348, 1349, 1350, 1351, 1352, 1353, 1354, 1355, 1356, 1357, 1358, 1359, 1360, 1361, 1362, 1363, 1364, 1365, 1366, 1367, 1368, 1369, 1370, 1371, 1372, 1373, 1374, 1375, 1376, 1377, 1378, 1379, 1380, 1381, 1382, 1383, 1384, 1385, 1386, 1387, 1388, 1389, 1390, 1391, 1392, 1393, 1394, 1395, 1396, 1397, 1398, 1399, 1400, or any range derivable therein, base pairs. This sigma factor can be easily identified due to the antisigma factor encoded just downstream having these distinguishing features. In some aspects, the anti-sigma factor comprises a beta-barrel outer membrane structure at the C-terminus, a periplasmic region, and an inner membrane domain at near the N-terminus. The associated antisigma factor refers to an antisigma factor having a beta-barrel outer membrane structure at the C-terminus. Examples of these anti-sigma factors are provided as SEQ ID NOS:4-8 and FIG. 11. The ECF-type sigma factor-induced gene includes genes that are upregulated by the sigma factor and/or synthesize a long O-antigen. In some aspects, the ECF-type sigma factor-induced gene refers to genes that make the long O-antigen of LPS.

Also described is a method comprising administering BcpT or BSAP-3 to bacterial cells or the cells of the disclosure. The methods may increase or further increase the cells' potential to engraft into the gut of mammalian systems. Further aspects relate to bacterial cells and populations of cells made by methods of the disclosure. Further aspects relate to compositions comprising cells of the disclosure. The compositions may comprise or further comprise a carrier, such as a pharmaceutical carrier.

Further method aspects relate to a method comprising administering the composition of the disclosure to a subject in need thereof. Also described is a method for increasing bacterial populations in the gut of a subject comprising administering a composition of the disclosure to a subject in need thereof. Yet further aspects relate to a method for increasing the engraftment of bacterial populations in the gut of a subject comprising administering a composition of the disclosure to a subject in need thereof. Also described are methods of treating a patient. In some aspects, the patient has an infection. Also described are methods for reversing adverse health outcomes, including adverse health outcomes associated with bacterial imbalances. Any of the methods can comprise 1, 2, 3, 4, 5, 6 or more of any of the following steps: measuring a bacterial population in a patient, measuring a microbiome profile in a patient, analyzing a biological sample from a patient, modifying a bacterial cell or population of cells, administering a bacterial cell or population of cells (which may be at one or more of the bacterial cell or population of cells disclosed herein), or monitoring a patient for bacterial composition changes.

In some aspects, the cells or cell comprises a deletion of a functional ECF-type-associated anti-sigma factor and deletion of a functional endogenous ECF-type sigma factor. In some aspects, the cell(s) are further classified as Gram negative bacterial cells. In some aspects, the bacterial cell is in the order of Bacteroidales. In some aspects, the bacterial cell is of the genus Prevotella, Phocaeicola, Alistipes, Parabacteroides, Odoribacter, or Bacteroides. It is specifically contemplated that, in some aspects, the bacterial cell is not of one or more of the genera Prevotella, Phocaeicola, Alistipes, Parabacteroides, Odoribacter, or Bacteroides.

In some aspects, the ECF-type sigma factor, functional fragment thereof, associated anti-sigma factor, or sigma factor-induced gene is from P. vulgatus or P. dorei. The cell(s) may be from Phocaeicola vulgatus, and comprise a deletion of a functional ECF-type-associated anti-sigma factor of SEQ ID NO:2. In some aspects, the heterologous nucleic acid encodes for a sigma factor comprising the amino acid sequence of one of SEQ ID NO:1 or 95-99 or an amino acid sequence with at least 70% sequence identity or similarity to one of SEQ ID NO:1 or 95-99. In some aspects, the heterologous nucleic acid encodes for a sigma factor having or having at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or any range derivable therein, percent sequence identity or similarity (or any derivable range therein) to one of SEQ ID NO:1 or 95-99. In some aspects, the nucleic acid encodes for a protein comprising one or more amino acid substitutions relative to one of SEQ ID NO:1 or 95-99. In some aspects, the one or more amino acid substitutions are conservative substitutions. In some aspects, the heterologous nucleic acid is on a plasmid. Alternatively, the heterologous nucleic acid may be inserted into the bacteria's genome.

The ECF-type sigma factor may comprise a sigma factor in the same operon as an anti-sigma factor comprising a beta-barrel outer-membrane structure. The antisigma factor may comprise an amino acid sequence of one of SEQ ID NOS:4-8 or an amino acid sequence with at least 70% sequence identity or similarity to one of SEQ ID NOS:4-8. In some aspects, the antisigma factor comprises an amino acid sequence having or having at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or any range derivable therein, percent sequence identity or similarity (or any derivable range therein) to one of SEQ ID NOS:4-8. In some aspects, the antisigma factor comprises a beta-barrier outer-membrane structure that is structurally similar to the beta-barrel outer membrane structure of SEQ ID NOS:4-8.

The ECF-type sigma factor-induced gene may comprise a gene encoding a protein with a NigD-like domain (pfam17415 or pfam12667) or ortholog thereof. In some aspects, the ECF-type sigma factor-induced gene comprises a gene selected from MON98_03293, MON98_09762, MON98_02415, MON98_01358, MON98_02916, MON98_01737, MON98_02416, MON98_00321, MON98_02417, MON98_01738, MON98_01576, and/or MON98_01669 and orthologs thereof. In some aspects, the ECF-type sigma factor-induced gene encodes for a protein of one of SEQ ID NOS:9-16 or an amino acid sequence with at least 70% sequence identity or similarity to one of SEQ ID NOS:9-16. In some aspects, the ECF-type sigma factor-induced gene encodes for a protein having an amino acid sequence having or having at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or any range derivable therein, percent sequence identity or similarity (or any derivable range therein) to one of SEQ ID NOS:9-16. In some aspects, the nucleic acid encodes for a protein comprising one or more amino acid substitutions relative to one of SEQ ID NOS:9-16. In aspects of the disclosure, the amino acid substitutions are conservative substitutions. In some aspects, the ECF-type sigma factor-induced gene comprises a gene selected from M0N98_00584, M0N98_03293, M0N98_00587, M0N98_00589, M0N98_02415, M0N98_02427, M0N98_03734, M0N98_00445, M0N98_01489, M0N98_02425, M0N98_00265, M0N98_02426, M0N98_00782, M0N98_01605, M0N98_00239, M0N98_02430, M0N98_03606, M0N98_04006, M0N98_03761, M0N98_01702, M0N98_02454, M0N98_01269, M0N98_00697, M0N98_01581, M0N98_04150, M0N98_01473, M0N98_00875, M0N98_03760, M0N98_01586, M0N98_02161, M0N98_00006, M0N98_02497, M0N98_00300, M0N98_00005, M0N98_03981, M0N98_00215, M0N98_00240, M0N98_00234, M0N98_00232, M0N98_00213, M0N98_00211, M0N98_02131, M0N98_02000, M0N98_03357, M0N98_01585, M0N98_01610, M0N98_00352, M0N98_00251, M0N98_01950, M0N98_01589, M0N98_01571, M0N98_00853, M0N98_04091, M0N98_01611, M0N98_04078, M0N98_00351, M0N98_03282, M0N98_00236, M0N98_03341, M0N98_00229, M0N98_02428, M0N98_00224, M0N98_02424, M0N98_00985, M0N98_01582, M0N98_00218, M0N98_02809, M0N98_01056, M0N98_00209, M0N98_04079, M0N98_02432, M0N98_03736, M0N98_00876, M0N98_01579, M0N98_01423, M0N98_02504, M0N98_00204, M0N98_02991, M0N98_00962, M0N98_02994, M0N98_00242, M0N98_03588, M0N98_01358, M0N98_04217, M0N98_03982, M0N98_04036, M0N98_03663, M0N98_03257, M0N98_03762, M0N98_03267, M0N98_03345, M0N98_00223, M0N98_00288, M0N98_00241, M0N98_03725, M0N98_00339, M0N98_00226, M0N98_02498, M0N98_00214, M0N98_01800, and orthologs and combinations thereof.

The cells may comprise or further comprise a heterologous nucleic acid encoding a transgene. The transgene may be an enzyme, a cytotoxic molecule, a therapeutic molecule, or an antimicrobial molecule. The cells may comprise or further comprise a protein or fragment thereof expressed from the heterologous nucleic acid. Nucleic acids may include RNA or DNA. In some aspects, the nucleic acid is a DNA molecule.

In the method aspects of the disclosure, the BcpT or BSAP-3 may be administered in a concentration of 0.1-200 μg/ml. In some aspects, BcpT or BSAP-3 is administered in a concentration of 1-20 μg/ml. The concentration administered refers to the concentration of the BcpT or BSAP-3 protein that is in contact with the cells, such as the concentration of the BcpT or BSAP-3 in a composition comprising the cells. In some aspects, the concentration of the administered BcpT or BSAP-3 is, is at least, or is at most 0.1, 0.5, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110,111, 112, 113, 114, 115, 116, 117, 118, 119, 120,121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, or any range derivable therein, μg/ml.

The cells of the disclosure may be further defined as live cells. The compositions may be formulated as a tablet, capsule, or food product. The subject may be a human subject. In some aspects, the subject is a human, mouse, rabbit, dog, cat, horse, rat, or laboratory test animal. In some aspects, the subject is a healthy subject. In some aspects, the subject is one that is suffering from inflammation in the gut. In some aspects, the subject is suffering from irritable bowel syndrome, inflammatory bowel disease, colitis, constipation, hemorrhoids, colon cancer, or a cancer of the digestive system.

In certain aspects the nucleic acid encodes for a protein or polypeptide (wild-type or modified) that may comprise, but is not limited to, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 275, 300, 325, 350, 375, 400, 425, 450, 475, 500, 525, 550, 575, 600, 625, 650, 675, 700, 725, 750, 775, 800, 825, 850, 875, 900, 925, 950, 1000, 1200, 1400, 1600, 1800, or 2000 amino acid residues and any range derivable therein. It is contemplated that the nucleic acids may encode for polypeptides that are mutated by truncation, rendering them shorter than their corresponding wild-type form, also, they might be altered by fusing or conjugating a heterologous protein or polypeptide sequence with a particular function (e.g., for targeting or localization, for enhanced immunogenicity, for purification purposes, etc.).

The nucleic acids may encode for polypeptides or proteins that may include 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 (or any derivable range therein) or more variant amino acids or nucleic acid substitutions or be at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% (or any derivable range therein) similar, identical, or homologous to at least, or at most 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 300, 400, 500, 550, 1000 or more contiguous amino acids or nucleic acids, or any range derivable therein, of SEQ ID NOS:1-17 or 95-99.

The nucleic acids of the disclosure may encode for polypeptides having, having at least, or having at most 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, or 615 substitutions (or any range derivable therein).

The substitution may be at amino acid position 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, or 650 of any of SEQ ID NOS:1-17 or 95-99 (or any derivable range therein) and may be a substitution with any amino acid or may be a substitution with a alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, or valine.

In some aspects, the nucleic acid encodes for a protein or polypeptide that comprises amino acids 1 to 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, or 320 (or any derivable range therein) of SEQ ID NOS:1-17 or 95-99.

In some aspects the nucleic acid encodes for a protein or polypeptide comprising amino acids 1 to 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, or 320 (or any derivable range therein) of SEQ ID NOS:1-17 or 95-99 and have or have at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% (or any derivable range therein) sequence identity or similarity to one of SEQ ID NOS:1-17 or 95-99.

In some aspects, the nucleic acid encodes for a protein or polypeptide comprising, comprising at least, or comprising at most 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, or 320 (or any derivable range therein) contiguous amino acids of SEQ ID NOS:1-17 or 95-99.

In some aspects, the nucleic acid encodes for a protein or polypeptide comprising, comprising at least, or comprising at most 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, or 320 (or any derivable range therein) contiguous amino acids of SEQ ID NOS:1-17 or 95-99 that are at least, at most, or exactly 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% (or any derivable range therein) similar, identical, or homologous to one of SEQ ID NOS:1-17 or 95-99.

In some aspects there is a nucleic acid molecule encoding for a polypeptide starting at position 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, or 950 (or any range derivable therein) of any of SEQ ID NOS:1-17 or 95-99 and comprising at least, at most, or exactly 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107,108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, or 950 (or any derivable range therein) contiguous amino acids of any of SEQ ID NOS:1-17 or 95-99.

The nucleotide as well as the protein, polypeptide, and peptide sequences for various genes have been previously disclosed, and may be found in the recognized computerized databases. Two commonly used databases are the National Center for Biotechnology Information's Genbank and GenPept databases (on the World Wide Web at ncbi.nlm.nih.gov/) and The Universal Protein Resource (UniProt; on the World Wide Web at uniprot.org). The coding regions for these genes may be amplified and/or expressed using the techniques disclosed herein or as would be known to those of ordinary skill in the art.

The following is a discussion of changing the amino acid subunits of a protein to create an equivalent, or even improved, second-generation variant polypeptide or peptide. For example, certain amino acids may be substituted for other amino acids in a protein or polypeptide sequence with or without appreciable loss of interactive binding capacity with structures such as, for example, antigen-binding regions of antibodies or binding sites on substrate molecules. Since it is the interactive capacity and nature of a protein that defines that protein's functional activity, certain amino acid substitutions can be made in a protein sequence and in its corresponding DNA coding sequence, and nevertheless produce a protein with similar or desirable properties. It is thus contemplated by the inventors that various changes may be made in the DNA sequences of genes which encode proteins without appreciable loss of their biological utility or activity.

The term “functionally equivalent codon” is used herein to refer to codons that encode the same amino acid, such as the six different codons for arginine. Also considered are “neutral substitutions” or “neutral mutations” which refers to a change in the codon or codons that encode biologically equivalent amino acids.

Amino acid sequence variants of the disclosure can be substitutional, insertional, or deletion variants. A variation in a polypeptide of the disclosure may affect 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or more non-contiguous or contiguous amino acids of the protein or polypeptide, as compared to wild-type (or any range derivable therein). A variant can comprise an amino acid sequence that is at least 50%, 60%, 70%, 80%, or 90%, including all values and ranges there between, identical to any sequence provided or referenced herein. A variant can include 2, 3, 4, 5, 6, 7, 8, 9, 10, 11, 12, 13, 14, 15, 16, 17, 18, 19, 20, or more substitute amino acids.

It also will be understood that amino acid and nucleic acid sequences may include additional residues, such as additional N- or C-terminal amino acids, or 5′ or 3′ sequences, respectively, and yet still be essentially identical as set forth in one of the sequences disclosed herein, so long as the sequence meets the criteria set forth above, including the maintenance of biological protein activity where protein expression is concerned. The addition of terminal sequences particularly applies to nucleic acid sequences that may, for example, include various non-coding sequences flanking either of the 5′ or 3′ portions of the coding region.

Deletion variants typically lack one or more residues of the native or wild type protein. Individual residues can be deleted or a number of contiguous amino acids can be deleted. A stop codon may be introduced (by substitution or insertion) into an encoding nucleic acid sequence to generate a truncated protein.

Insertional mutants typically involve the addition of amino acid residues at a non-terminal point in the polypeptide. This may include the insertion of one or more amino acid residues. Terminal additions may also be generated and can include fusion proteins which are multimers or concatemers of one or more peptides or polypeptides described or referenced herein.

Substitutional variants typically contain the exchange of one amino acid for another at one or more sites within the protein or polypeptide, and may be designed to modulate one or more properties of the polypeptide, with or without the loss of other functions or properties. Substitutions may be conservative, that is, one amino acid is replaced with one of similar chemical properties. “Conservative amino acid substitutions” may involve exchange of a member of one amino acid class with another member of the same class. Conservative substitutions are well known in the art and include, for example, the changes of: alanine to serine; arginine to lysine; asparagine to glutamine or histidine; aspartate to glutamate; cysteine to serine; glutamine to asparagine; glutamate to aspartate; glycine to proline; histidine to asparagine or glutamine; isoleucine to leucine or valine; leucine to valine or isoleucine; lysine to arginine; methionine to leucine or isoleucine; phenylalanine to tyrosine, leucine or methionine; serine to threonine; threonine to serine; tryptophan to tyrosine; tyrosine to tryptophan or phenylalanine; and valine to isoleucine or leucine. Conservative amino acid substitutions may encompass non-naturally occurring amino acid residues, which are typically incorporated by chemical peptide synthesis rather than by synthesis in biological systems. These include peptidomimetics or other reversed or inverted forms of amino acid moieties.

Alternatively, substitutions may be “non-conservative”, such that a function or activity of the polypeptide is affected. Non-conservative changes typically involve substituting an amino acid residue with one that is chemically dissimilar, such as a polar or charged amino acid for a nonpolar or uncharged amino acid, and vice versa. Non-conservative substitutions may involve the exchange of a member of one of the amino acid classes for a member from another class.

One skilled in the art can determine suitable variants of polypeptides as set forth herein using well-known techniques. One skilled in the art may identify suitable areas of the molecule that may be changed without destroying activity by targeting regions not believed to be important for activity. The skilled artisan will also be able to identify amino acid residues and portions of the molecules that are conserved among similar proteins or polypeptides. In further aspects, areas that may be important for biological activity or for structure may be subject to conservative amino acid substitutions without significantly altering the biological activity or without adversely affecting the protein or polypeptide structure.

In making such changes, the hydropathy index of amino acids may be considered. The hydropathy profile of a protein is calculated by assigning each amino acid a numerical value (“hydropathy index”) and then repetitively averaging these values along the peptide chain. Each amino acid has been assigned a value based on its hydrophobicity and charge characteristics. They are: isoleucine (+4.5); valine (+4.2); leucine (+3.8); phenylalanine (+2.8); cysteine/cysteine (+2.5); methionine (+1.9); alanine (+1.8); glycine (−0.4); threonine (−0.7); serine (−0.8); tryptophan (−0.9); tyrosine (−1.3); proline (1.6); histidine (−3.2); glutamate (−3.5); glutamine (−3.5); aspartate (−3.5); asparagine (−3.5); lysine (−3.9); and arginine (−4.5). The importance of the hydropathy amino acid index in conferring interactive biologic function on a protein is generally understood in the art (Kyte et al., J. Mol. Biol. 157:105-131 (1982)). It is accepted that the relative hydropathic character of the amino acid contributes to the secondary structure of the resultant protein or polypeptide, which in turn defines the interaction of the protein or polypeptide with other molecules, for example, enzymes, substrates, receptors, DNA, antibodies, antigens, and others. It is also known that certain amino acids may be substituted for other amino acids having a similar hydropathy index or score, and still retain a similar biological activity. In making changes based upon the hydropathy index, in certain aspects, the substitution of amino acids whose hydropathy indices are within ±2 is included. In some aspects of the invention, those that are within ±1 are included, and in other aspects of the invention, those within ±0.5 are included.

It also is understood in the art that the substitution of like amino acids can be effectively made based on hydrophilicity. U.S. Pat. No. 4,554,101, incorporated herein by reference, states that the greatest local average hydrophilicity of a protein, as governed by the hydrophilicity of its adjacent amino acids, correlates with a biological property of the protein. In certain aspects, the greatest local average hydrophilicity of a protein, as governed by the hydrophilicity of its adjacent amino acids, correlates with its immunogenicity and antigen binding, that is, as a biological property of the protein. The following hydrophilicity values have been assigned to these amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); and tryptophan (−3.4). In making changes based upon similar hydrophilicity values, in certain aspects, the substitution of amino acids whose hydrophilicity values are within ±2 are included, in other aspects, those which are within ±1 are included, and in still other aspects, those within ±0.5 are included. In some instances, one may also identify epitopes from primary amino acid sequences based on hydrophilicity. These regions are also referred to as “epitopic core regions.” It is understood that an amino acid can be substituted for another having a similar hydrophilicity value and still produce a biologically equivalent and immunologically equivalent protein.

Additionally, one skilled in the art can review structure-function studies identifying residues in similar polypeptides or proteins that are important for activity or structure. In view of such a comparison, one can predict the importance of amino acid residues in a protein that correspond to amino acid residues important for activity or structure in similar proteins. One skilled in the art may opt for chemically similar amino acid substitutions for such predicted important amino acid residues.

One skilled in the art can also analyze the three-dimensional structure and amino acid sequence in relation to that structure in similar proteins or polypeptides. In view of such information, one skilled in the art may predict the alignment of amino acid residues of an antibody with respect to its three-dimensional structure. One skilled in the art may choose not to make changes to amino acid residues predicted to be on the surface of the protein, since such residues may be involved in important interactions with other molecules. Moreover, one skilled in the art may generate test variants containing a single amino acid substitution at each desired amino acid residue. These variants can then be screened using standard assays for binding and/or activity, thus yielding information gathered from such routine experiments, which may allow one skilled in the art to determine the amino acid positions where further substitutions should be avoided either alone or in combination with other mutations. Various tools available to determine secondary structure can be found on the world wide web at expasy.org/proteomics/protein structure.

In some aspects of the invention, amino acid substitutions are made that: (1) reduce susceptibility to proteolysis, (2) reduce susceptibility to oxidation, (3) alter binding affinity for forming protein complexes, (4) alter ligand or antigen binding affinities, and/or (5) confer or modify other physicochemical or functional properties on such polypeptides. For example, single or multiple amino acid substitutions (in certain aspects, conservative amino acid substitutions) may be made in the naturally occurring sequence. Substitutions can be made in that portion of the antibody that lies outside the domain(s) forming intermolecular contacts. In such aspects, conservative amino acid substitutions can be used that do not substantially change the structural characteristics of the protein or polypeptide (e.g., one or more replacement amino acids that do not disrupt the secondary structure that characterizes the native antibody).

The compositions of the disclosure may exclude one or more bacteria genera or species described herein or may include less than 1×10⁶, 1×10⁵, 1×10⁴, 1×10³, or 1×10² cells or CFU (or any derivable range therein) of one or more of the bacteria described herein.

In some embodiments, each of the populations of bacteria is present in the composition at a concentration of at least 1×10³ CFU. In some embodiments, the composition is a live bacterial product or a live biotherapeutic product. In some embodiments, the bacteria are lyophilized, freeze dried, or frozen. In some embodiments, the composition is formulated for oral delivery. In some embodiments, the composition formulated for oral delivery is a tablet or capsule. In some embodiments, the tablet or capsule comprises an acid-resistant enteric coating. In some embodiments, the composition is formulated for administration rectally, via colonoscopy, sigmoidoscopy by nasogastric tube, or enema. In some embodiments, the composition is capable of being re-formulated for final delivery as comprising a liquid, a suspension, a gel, a geltab, a semisolid, a tablet, a sachet, a lozenge, a capsule, or as an enteral formulation. In some embodiments, the composition is formulated for multiple administrations. In some embodiments, the composition further comprises a pharmaceutically acceptable excipient.

It is specifically contemplated that any limitation discussed with respect to one embodiment of the invention may apply to any other embodiment of the invention. Furthermore, any composition of the invention may be used in any method of the invention, and any method of the invention may be used to produce or to utilize any composition of the invention. Aspects of an embodiment set forth in the Examples are also embodiments that may be implemented in the context of embodiments discussed elsewhere in a different Example or elsewhere in the application, such as in the Summary of Invention, Detailed Description of the Embodiments, Claims, and description of Figure Legends.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1. Agar spot overlay assays of P. vulgatus and P. dorei strains. Upper panel: pattern of the 18 strains (three down, six across) that were spotted onto the plate and grown overnight to test for production of growth inhibitory factor(s) of the overlaid strains. Strains listed in red font produce BSAP-3, the strain listed in blue font produces bacteriodetocin A. Lower panel: The strain names on the top or bottom of the plates indicate the strain that was used as the overlay strain to test for growth inhibition by product(s) secreted by the strains listed above. All overlay assays were performed a minimum of four times with similar results.

FIG. 2A-H. Identification of the toxin-encoding gene on a mobile plasmid. a. Agar overlay assays of toxin producing strain PvCL04 and two transposon mutants that abrogate its ability to inhibit the growth of PvCL10 and PvCL09. b. Genetic context of the gene into which the two transposons inserted. c. Agar overlay assays showing that deletion of the gene identified by transposon mutagenesis from strains PvCL04 and PdCL02 abrogates toxin activity, which is restored when the gene is added back in trans to these strains. d. Agar spot overlay assay showing that placement of CS034_04058 in trans in B. thetaiotaomicron VPI 5482 confers toxin activity to the strain. e. Orf map of the contig of the sequenced genome of PvCL04 containing CS034_04058. Primers used to determine if the contig is a circular plasmid and to identify the toxin producing gene in other strains are shown. Sites of the transposon insertions of mutants Tn10 and Tn12 are shown. An insertion sequence present in a similar contig of strain PvCL10 is shown under the map with its insertion site mapped relative to the contig of PvCL04. f. EtBr-stained gel showing the results of the PCR using the primers shown in panel E with each of the 18 P. vulgatus and P. dorei strains listed in FIG. 1a. PCR analyses were performed twice. g. The circular 9117 pBCPT plasmid of strain PvCL04 showing the portion of the contig that is similar to the Bacteroidales mobile plasmid pBUN24 (pink outer line). The gene shown in green encodes a plasmid replication protein, the genes in blue encode mobilization proteins, and the genes in orange encode a putative toxin-antitoxin system. h. Overlay assays showing that B. fragilis □T6SSermG and B. ovatus D2 that conjugally received pBCPT-tetQ-ttr from PvCL04 pBCPT-tetQ-ttr acquire the ability to inhibit the growth of PvCL10. All overlay assays were performed at least twice.

FIG. 3A-H. BcpT requires proteolytic cleavage for activity. a. Agar overlay assays using PvCL10 as the overlay strain with 450 ng His-BcpT, or His-BcpT cleaved with Factor Xa, or Factor Xa alone. b. Coomassie-stained polyacrylamide gel of His-BcpT cleaved with Factor Xa for various periods of time showing cleavage products a, b and c. c. Western immunoblot of supernatants of overnight cultures of the indicated strain or mutant probed with α-BcpT showing a representative of three independent blots. d. Sequence of BcpT with the native signal sequence showing the SpII cleavage site and the sequence of the construct with the His-tag. The N-terminal sequences of the a, b, and c fragments of panel B determined by amino acid sequencing are boxed. Fragment a is colored blue and fragment c is colored green. Fragment b is the combination of the beige and green sequences. e. Coomassie stained gels of recombinant BcpT with mutants at LTR sites, R65N and R199N, cleaved with FXa. The (*) indicates the presence of a probable protein from E. coli in that lane and the following 3 lanes. f. Agar overlay assays using PvC110 were spotted with the purified BcpT WT and activation site mutants. The horizontal and vertical hashmarks are from the polystyrene plate grids. g. Western blot of supernatants of overnight cultures of the reconstructed BcpT site mutants R65N, R199N, R65RN199N in the PdCL02 ΔbcpT background probed with α-BcpT. h Agar overlay assays using PvCL10 as the overlay strain with WT PdCL02 and each of the PdCL02 reconstructed BcpT site mutants in the PdCL02 ΔbcpT background. The (*) indicates reconstructed plasmid. All overlay assays, Coomassie-stained gels and western blots were performed at least twice.

FIG. 4A-E. C11 family proteases of P. dorei can activate BcpT. a. Left panel: Coomassie-stained gel of purified recombinant doripains (Dpns) and their ability to cleave BcpT. Purified recombinant doripain A (DpnA) incubated with trypsin at a 1:50 (w:w) trypsin to DpnA ratio for 15 mins at 37° C. was sufficient to cleave the activation loop without degradation of the protein. DpnA activated in this manner is referred to as DpnA(a). Red boxes indicate cleaved doripain B (DpnB) fragments that eluted with contaminating proteins from E. coli, possibly due to their association with the active protease Right panel: FXa and DpnB cleaved BcpT showing the same sized fragments are generated. Arrows indicate the DpnB protease fragments that result from its activation b. Agar overlay assays showing that the Dpns can activate BcpT like FXa. and inhibit the growth of Pv8482. Dpns did not activate the R65N or the R199N-R65N double mutants, however, a weak activation of the R199N was observed. Although this assay is only semi-quantitiative the spot of the undiluted R199N (5 g) was most closely resembled that of 0.04 g the WT activity, which is less than 10% of WT. c. Western immunoblot of purified BcpT and site mutants of BcpT cleaved by DpnA(a) and DpnB. The blot was probed with α-BcpT. Note that the smaller fragment in the R65N in the DpnA is smaller than that of the DpnB cleaved R65N mutant. We have found that DpnA cleaves the His Tag more efficiently than DpnB which accounts for the difference in size of the small fragment of ˜2.5 kDa. d. Western immunoblot analysis of BcpT cleavage products in the supernatant of PdCL02 and dpn mutants and complemented mutants. e. Agar overlay assay analyzing the ability of PdCL02 dpn mutants to inhibit the growth of PvCL10. Overlays and BcpT cleavage analyses were performed two to three times with consistent results. All gels and blots were performed a minimum of two times with consistent results.

FIG. 5A-E. Identification of BcpT receptor. a. Receptor blots of membranes of Pv8482 WT and O-antigen mutant probed with unactivated or activated BcpT and detected with affinity purified α-BcpT. b. Receptor blot of purified LPS from the indicated strains probed with Alexa 488 labeled activated BcpT. c. Overlay assays of resistance of O-antigen mutants of PvCL10 and By 8482 to purified BcpT. d. Agar overlay assays showing the sensitivity of various Bacteroidales species and strains to BcpT. The left most spot of each overlay is 1 μg of toxin and each subsequent spot is a two-fold dilution. e. Agar spot overlay assays showing the ability of each of three pBCPT genes to protect PvCL10 from BcpT toxicity when expressed in trans. Overlay assays were performed twice and receptor probing blots were performed twice with consistent results.

FIG. 6A-G. A BcpT-induced protective regulon. a. Agar spot overlay assays of four transposon (Tn) mutants of PvCL10 that were slightly protected from BcpT and substantial protected from BSAP-3. b. Orf map of the anti-sigma factor encoding gene (MON98_03760) into which all four transposons inserted showing the genetic context of the upstream gene encoding the cognate sigma factor (MON98_03761). Bottom panel shows the regions of the anti-sigma factor including the inner transmembrane region shown in red and the outer membrane β-barrel region and sites of each of the four transposon mutants. c. Agar spot overlay assays showing the sensitivity of the mutant strains of PvCL10 (indicated on right) to each of the toxin producing strains or the toxin deletion mutants. d. Western immunoblot showing LPS O-antigen sizes of various PvCL10 WT and mutant strains. Probed with antiserum to PvCL10 O-antigen. e. Western immunoblot of whole cell lysates of PvCL10 treated with two concentrations of BcpT for three hours in duplicate or OD₆₀₀ matched untreated controls. Bacterial equivalent to 4 μl of the culture were added to each well. The blot is probed with antiserum to the PvCL10 O-antigen. f. List of the 14 genes that are differentially expressed using the criteria listed in the methods section in PvCL10 tn79 compared to WT PvCL10. Green indicates upregulation and red indicates downregulation. FC represents the fold-change and padj represents the p-value adjusted for multiple comparisons by DESeq2. All 14 genes are also differentially expressed when PvCL10 is treated with BcpT. g. Agar spot overlay assays showing the susceptibility of various strains to dilutions of BSAP-3 and BcpT. The first spot of the BSAP-3 plates is 1 μg and the first spot of the BcpT plates is 3 μg and each consecutive spot is a 2-fold dilution left to right on the top row and then left to right on the bottom row. Overlay assays and western immunoblots were performed at least twice with consistent results.

FIG. 7A-F. Analysis of pBCPT plasmid mobilization. a. Plasmid map of pBCPT with gene names from PvCL04. The insertion site of tetQ and the transcriptional termination region (ttr) is shown. b. Amplified intergenic region between CS034_04061 and CS034_04061 showing that the insertion does not affect the promoters of either divergently transcribed gene. c. d. Plates showing the colonies arising on erythromycin plates (recipient strain) and tetracycline/erythromycin plates (transconjugants) for transfer of the pBCPT-tetQ plasmid from PvCL04 to B. fragilis 638RermG_T6SS (C) and B. ovatus D2 (D). e. f. Amplicons resulting from PCR with primers specific to the bcpT gene (top) and a multiplex of the 16S rRNA gene (bottom) of the transconjugants.

FIG. 8. Trypsin digestion of BcpT cleavage site mutants. Purified recombinant wt BcpT and its cleavage site mutants, R65N, R199N and R65N-R199N, were cleaved (5 μg in each reaction) with chymotrypsin. Two-fold dilutions of chymotrypsin starting with 2.5 μg of chymotrypsin were generated in HBS and to each dilution 5 μg of BcpT was added (final reaction volume of 20 μl) and incubated for 30 min at 37° C. The digestion reactions were stopped by the addition of 5 μl of SOS-PAGE sample buffer and heating to 95° C. for 5 min. The cleavage products were separated on an 8-16% SOS-PAGE: the cleavage products are shown in lanes 1-10. Lane 11 contains uncleaved WT BcpT or its derivatives. Lane 12 contains 2.5 μg of chymotrypsin.

FIG. 9A-E. Analysis of BcpT and BcpT site mutants binding to components of membrane fraction and LPS of indicated strains on Western blots. a. Receptor blot of membranes from Bv8482 or its O-antigen mutant probed with BcpT site mutants (see Methods for receptor blots details). b. Silver-stain analysis of LPS extracts from indicated strains. c. Probing of blots containing purified LPS with Alexa-488-labeled BcpT. d. Spot tests of the activated WT BcpT and the Alexa-fluor labeled WT BcpT use to probe the lipid blots in panel e and in FIG. 5. e. Spot tests of activated BcpT (5 μg) on the strains of E. coli and Salmonella enterica.

FIG. 10A-C. Amino terminal fragments of BcpT block BcpT binding to membrane LPS. a. Receptor blot of Bv8482 membranes probed with FXa-cleaved BcpT (1.5 μg/ml) (see Methods for details). b. Receptor blot of Bv8482 membranes probed with FXa-cleaved BcpT in the presence of 20-fold molar excess N-terminal BcpT fragment 18-199. c. Receptor blot of Bv8482 membranes probed with FXa-cleaved BcpT in the presence of 20-fold molar excess N-terminal BcpT fragment 66-199.

FIG. 11A-F shows the predicted structure of the anti-sigma factor in various bacterial species using the program alpha-fold. The antisigma factors are identified by their large size and the beta-barrel outer membrane structure with a long unstructured periplasmic region and then an inner membrane spanning domain at the N-terminus. There is one of each of these anti-sigma factor encoded gene in each Bacteroidales genome. Included below are structures from diverse gut Bacteroidales species including a) Phocaeicola vulgatus, b) Bacteroides salyersiae, c) Bacteroides uniformis, d) Parabacteroides merdae, e) Prevotella copri, and f) Bacteroides caccae, which showing a conserved structure.

### DESCRIPTION OF ILLUSTRATIVE EMBODIMENTS

The inventors found that exposure of sensitive cells to BcpT induces activation of a sigma/antisigma factor pair whose regulon includes genes involved in the synthesis of a protective response to two antibacterial toxins, BcpT and BSAP-3. BcpT defines a previously unidentified family of antibacterial toxins that is widespread in human gut microbiomes. It is hypothesized that administering the toxin or mimicking the administration of the toxin through genetic manipulations of the bacteria to induce or turn on expression of the sigma factor, may increase the potential of the bacteria to engraft into the gut of mammalian systems.

### I. Definitions

The phrases “pharmaceutical composition” or “pharmacologically acceptable composition” refers to molecular entities and compositions that do not produce an adverse, allergic, or other untoward reaction when administered to an animal, such as a human, as appropriate. The preparation of a pharmaceutical composition comprising an antibody or additional active ingredient will be known to those of skill in the art in light of the present disclosure.

As used herein, “pharmaceutically acceptable carrier” includes any and all aqueous solvents (e.g., water, alcoholic/aqueous solutions, saline solutions, parenteral vehicles, such as sodium chloride, and Ringer's dextrose), non-aqueous solvents (e.g., propylene glycol, polyethylene glycol, vegetable oil, and injectable organic esters, such as ethyloleate), dispersion media, coatings, surfactants, antioxidants, preservatives (e.g., antibacterial or antifungal agents, anti-oxidants, chelating agents, and inert gases), isotonic agents, absorption delaying agents, salts, drugs, drug stabilizers, gels, binders, excipients, disintegration agents, lubricants, sweetening agents, flavoring agents, dyes, fluid and nutrient replenishers, such like materials and combinations thereof, as would be known to one of ordinary skill in the art. The pH and exact concentration of the various components in a pharmaceutical composition may be adjusted according to well-known parameters.

The term “unit dose” or “dosage” refers to physically discrete units suitable for use in a subject, each unit containing a predetermined quantity of the therapeutic composition calculated to produce the desired responses discussed herein in association with its administration, i.e., the appropriate route and treatment regimen. The quantity to be administered, both according to number of treatments and unit dose, depends on the effect desired. The actual dosage amount of a composition of the present embodiments administered to a patient or subject can be determined by physical and physiological factors, such as body weight, the age, health, and sex of the subject, the type of disease being treated, the extent of disease penetration, previous or concurrent therapeutic interventions, idiopathy of the patient, the route of administration, and the potency, stability, and toxicity of the particular therapeutic substance. For example, a dose may also comprise from about 1 μg/kg/body weight to about 1000 mg/kg/body weight (this such range includes intervening doses) or more per administration, and any particular dose derivable therein. In non-limiting examples of a range derivable from the numbers listed herein, a range of about 5 μg/kg/body weight to about 100 mg/kg/body weight, about 5 μg/kg/body weight to about 500 mg/kg/body weight, etc., can be administered. The practitioner responsible for administration will, in any event, determine the concentration of active ingredient(s) in a composition and appropriate dose(s) for the individual subject.

A “population” of bacteria may refer to a composition of bacteria comprising a single species, or a mixture of different strains and species.

“Subject” and “patient” refer to either a human or non-human, such as primates, mammals, and vertebrates. In particular embodiments, the subject is a human.

As used herein, the terms “treat,” “treatment,” “treating,” “ameliorating”, or “amelioration” when used in reference to a disease, disorder or medical condition, refer to therapeutic treatments for a condition, wherein the object is to reverse, alleviate, ameliorate, inhibit, slow down or stop the progression or severity of a symptom or condition. The term is also applied to improving, by at least one measure, the gut microbiome health. The term “treating” includes reducing or alleviating at least one adverse effect or symptom of a condition or the reduction of a strain of bacteria or molecules that brings about a conditionally unfavorable state such as resistance to various drug treatments. Treatment is generally “effective” if one or more symptoms or clinical markers are reduced. Alternatively, treatment is “effective” if the progression of a condition is reduced or halted. That is, “treatment” includes not just the improvement of symptoms or markers, but also a cessation or at least slowing of progress or worsening of symptoms that would be expected in the absence of treatment. Beneficial or desired clinical results include, but are not limited to, alleviation of one or more symptom(s), diminishment of extent of the deficit, stabilized (i.e., not worsening) state of a tumor or malignancy, delay or slowing of tumor growth and/or metastasis, and an increased lifespan as compared to that expected in the absence of treatment.

The “gut microbiota” or “gut microbiome” designates the community of microorganisms (and their genomes) living in the intestine of a subject.

The term “heterologous” in the context of nucleic acid or protein refers to a non-endogenous nucleic acid (or protein expressed from the nucleic acid) that was transferred into the cell. The heterologous nucleic acid may be identical to an endogenous nucleic acid, but is still considered heterologous if it was transferred into the cell by a gene transfer method. Furthermore, nucleic acids in cells that are the progeny of parental cells comprising heterologous nucleic acids also comprise a heterologous nucleic acid if the nucleic acid is a replication from a nucleic acid in the parental cell that was heterologous (i.e. transferred into the parental cell by a gene transfer method).

The term “isolated” encompasses a bacterium or other entity or substance that has been (1) separated from at least some of the components with which it was associated when initially produced (whether in nature or in an experimental setting), and/or (2) produced, prepared, purified, and/or manufactured by the hand of man. Isolated bacteria may be separated from at least about 10%, about 20%, about 30%, about 40%, about 50%, about 60%, about 70%, about 80%, about 90%, or more of the other components with which they were initially associated. In some embodiments, isolated bacteria are more than about 80%, about 85%, about 90%, about 91%, about 92%, about 93%, about 94%, about 95%, about 96%, about 97%, about 98%, about 99%, or more than about 99% pure. As used herein, a substance is “pure” if it is substantially free of other components.

The terms “purify,” “purifying” and “purified” refer to a bacterium or other material that has been separated from at least some of the components with which it was associated either when initially produced or generated (e.g., whether in nature or in an experimental setting), or during any time after its initial production. A bacterium or a bacterial population may be considered purified if it is isolated at or after production, such as from a material or environment containing the bacterium or bacterial population, and a purified bacterium or bacterial population may contain other materials up to about 10%, about 20%, about 30%, about 40%, about 50%, about 60%, about 70%, about 80%, about 90%, or above about 90% and still be considered “isolated.” In some embodiments, purified bacteria and bacterial populations are more than about 80%, about 85%, about 90%, about 91%, about 92%, about 93%, about 94%, about 95%, about 96%, about 97%, about 98%, about 99%, or more than about 99% pure. In the instance of bacterial compositions provided herein, the one or more bacterial types present in the composition can be independently purified from one or more other bacteria produced and/or present in the material or environment containing the bacterial type. Bacterial compositions and the bacterial components thereof are generally purified from residual habitat products.

The term “determined to have” refers to a patient population that has been tested and reported as having a certain outcome, such as microbiome status.

The terms “lower,” “reduced,” “reduction,” “decrease,” or “inhibit” are all used herein generally to mean a decrease by a statistically significant amount. However, for avoidance of doubt, “lower,” “reduced,” “reduction, “decrease,” or “inhibit” means a decrease by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (i.e. absent level as compared to a reference sample), or any decrease between 10-100% as compared to a reference level.

The terms “increased,” “increase,” “enhance,” or “activate” are all used herein to generally mean an increase by a statically significant amount; for the avoidance of any doubt, the terms “increased,” “increase,” “enhance,” or “activate” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level.

The term “comprising,” which is synonymous with “including,” “containing,” or “characterized by,” is inclusive or open-ended and does not exclude additional, unrecited elements or method steps. The phrase “consisting of” excludes any element, step, or ingredient not specified. The phrase “consisting essentially of” limits the scope of described subject matter to the specified materials or steps and those that do not materially affect its basic and novel characteristics. The term permits the presence of additional elements that do not materially affect the basic and novel or functional characteristic(s) of that embodiment of the invention. With respect to pharmaceutical compositions, the term “consisting essentially of” includes the active ingredients recited, excludes any other active ingredients, but does not exclude any pharmaceutical excipients or other components that are not therapeutically active. It is contemplated that embodiments described in the context of the term “comprising” may also be implemented in the context of the term “consisting of” or “consisting essentially of.”

The term “consisting of” refers to compositions, methods, and respective components thereof as described herein, which are exclusive of any element not recited in that description of the embodiment.

As used in this specification and the appended claims, the singular forms “a”, “an”, and “the” include plural references unless the context clearly dictates otherwise. Thus for example, references to “the method” includes one or more methods, and/or steps of the type described herein and/or which will become apparent to those persons skilled in the art upon reading this disclosure and so forth.

As used herein, “essentially free,” in terms of a specified component, is used herein to mean that none of the specified component has been purposefully formulated into a composition and/or is present only as a contaminant or in trace amounts. The total amount of the specified component resulting from any unintended contamination of a composition is therefore well below 0.01%. Most preferred is a composition in which no amount of the specified component can be detected with standard analytical methods.

As used herein, the terms “or” and “and/or” are utilized to describe multiple components in combination or exclusive of one another. For example, “x, y, and/or z” can refer to “x” alone, “y” alone, “z” alone, “x, y, and z,” “(x and y) or z,” “x or (y and z),” or “x or y or z.” It is specifically contemplated that x, y, or z may be specifically excluded from an embodiment.

Throughout this application, the term “about” is used according to its plain and ordinary meaning in the area of microbiology to indicate that a value includes the standard deviation of error for the device or method being employed to determine the value.

### II. Microbial Modulators

The present disclosure also provides a pharmaceutical composition comprising one or more microbial populations as described above and at, for example, in the summary of the invention. The bacterial species therefore are present in the dose form as live bacteria, whether in dried, lyophilized, or sporulated form. This may be preferably adapted for suitable administration; for example, in tablet or powder form, potentially with an enteric coating, for oral treatment.

In particular aspects, the composition is formulated for oral administration. Oral administration may be achieved using a chewable formulation, a dissolving formulation, an encapsulated/coated formulation, a multi-layered lozenge (to separate active ingredients and/or active ingredients and excipients), a slow release/timed release formulation, or other suitable formulations known to persons skilled in the art. Although the word “tablet” is used herein, the formulation may take a variety of physical forms that may commonly be referred to by other terms, such as lozenge, pill, capsule, or the like.

While the compositions of the present disclosure are preferably formulated for oral administration, other routes of administration can be employed, however, including, but not limited to, subcutaneous, intramuscular, intradermal, transdermal, intraocular, intraperitoneal, mucosal, vaginal, rectal, and intravenous.

The desired dose of the composition of the present disclosure may be presented in multiple (e.g., two, three, four, five, six, or more) sub-doses administered at appropriate intervals throughout the day.

In one aspect, the disclosed composition may be prepared as a capsule. The capsule (i.e., the carrier) may be a hollow, generally cylindrical capsule formed from various substances, such as gelatin, cellulose, carbohydrate or the like.

In another aspect, the disclosed composition may be prepared as a suppository. The suppository may include but is not limited to the bacteria and one or more carriers, such as polyethylene glycol, acacia, acetylated monoglycerides, carnauba wax, cellulose acetate phthalate, corn starch, dibutyl phthalate, docusate sodium, gelatin, glycerin, iron oxides, kaolin, lactose, magnesium stearate, methyl paraben, pharmaceutical glaze, povidone, propyl paraben, sodium benzoate, sorbitan monooleate, sucrose talc, titanium dioxide, white wax and coloring agents.

In some aspects, the disclosed microbial modulator composition may be prepared as a tablet. The tablet may include the bacteria and one or more tableting agents (i.e., carriers), such as dibasic calcium phosphate, stearic acid, croscarmellose, silica, cellulose and cellulose coating. The tablets may be formed using a direct compression process, though those skilled in the art will appreciate that various techniques may be used to form the tablets.

In other aspects, the disclosed microbial modulator composition may be formed as food or drink or, alternatively, as an additive to food or drink, wherein an appropriate quantity of bacteria is added to the food or drink to render the food or drink the carrier.

The microbial modulator compositions of the present disclosure may further comprise one or more prebiotics known in the art, such as lactitol, inulin, or a combination thereof.

In some embodiments, the nutritional supplement is produced by a bacterium associated with a healthy human gut microbiome.

### III. Administration of Therapeutic Compositions

The compositions of the disclosure may be administered in any suitable manner known in the art. The compositions may be administered in doses of once a day, twice a day, or at a time period of, of at least, or of at most 1, 2, or 3 times a day or a week for, for at least, or for at most 1, 2, 3, 4, 5, 6, 7, 8, 9, 10, weeks or 1, 2, 3, 4, 5, 6, 7, 8, 9, 10, or 12 months.

In some aspects, the composition is formulated for oral administration. The skilled artisan knows a variety of formulas which can encompass living or killed microorganisms and which can present as food supplements (e.g., pills, tablets and the like) or as functional food such as drinks or fermented yogurts.

In some aspects, the compositions are administered, by implantation, intrathecally, intranasally, topically, or orally,

In some aspects, a particular dosage of bacteria, cells, or cellular populations are administered. In some aspects, a single dose will contain an amount of bacteria (such as a specific bacteria or species, genus, or family described herein) of at least, at most, or exactly 1×10⁴, 1×10⁵, 1×10⁶, 1×10⁷, 1×10⁸, 1×10⁹, 1×10¹⁰, 1×10¹¹, 1×10¹², 1×10¹³, 1×10¹⁴, 1×10¹⁵ or greater than 1×10¹⁵ CFU (or any derivable range therein) of a specified bacteria. In some embodiments, a single dose will contain at least, at most, or exactly 1×10⁴, 1×10⁵, 1×10⁶, 1×10⁷, 1×10⁸, 1×10⁹, 1×10¹⁰, 1×10¹¹, 1×10¹², 1×10¹³, 1×10¹⁴, 1×10¹⁵ or greater than 1×10¹⁵ CFU (or any derivable range therein) of total bacteria. In some embodiments, the composition comprises or the method comprises administration of at least, at most, or exactly 1, 2, 3, 4, 5, 6, 7, 8, 9, 10, 11, 12, 13, 14, 15, 20, 25, 30, 40, or 50 (or any derivable range therein) of different bacterial species, different bacterial genus, different bacterial family, or different bacterial isolated populations (such as different modified bacterial populations).

In some embodiments, the compositions comprise, comprise at most, or comprise at least about 1×10³ cells of bacteria or at least about 1×10⁴, 1×10⁵, 1×10⁶, 1×10⁷, 1×10⁸, 1×10⁹, 1×10¹⁰, 1×10¹¹, 1×10¹², 1×10¹³, 1×10¹⁴, 1×10¹⁵ cells (or any derivable range therein). In some embodiments, a single dose will contain an amount of bacteria (such as a specific bacteria or species, genus, family, or modified population described herein) of at least, at most, or exactly 1×10⁴, 1×10⁵, 1×10⁶, 1×10⁷, 1×10⁸, 1×10⁹, 1×10¹⁰, 1×10¹¹, 1×10¹², 1×10¹³, 1×10¹⁴, 1×10¹⁵ or greater than 1×10¹⁵ cells (or any derivable range therein) of a specified bacteria. In some embodiments, a single dose will contain at least, at most, or exactly 1×10⁴, 1×10⁵, 1×10⁶, 1×10⁷, 1×10⁸, 1×10⁹, 1×10¹⁰, 1×10¹¹, 1×10¹², 1×10¹³, 1×10¹⁴, 1×10¹⁵ or greater than 1×10¹⁵ cells (or any derivable range therein) of total bacteria.

The administration of the compositions may include various “unit doses.” Unit dose is defined as containing a predetermined-quantity of the therapeutic composition. The quantity to be administered, and the particular route and formulation, is within the skill of determination of those in the clinical arts. A unit dose need not be administered as a single injection but may comprise continuous infusion over a set period of time. In some embodiments, a unit dose comprises a single administrable dose.

The quantity to be administered, both according to number of treatments and unit dose, depends on the treatment effect desired. An effective dose is understood to refer to an amount necessary to achieve a particular effect. In the practice in certain embodiments, it is contemplated that doses in the range from 10 mg/kg to 200 mg/kg can affect the protective capability of these agents. Thus, it is contemplated that doses include doses of about 0.1, 0.5, 1, 5, 10, 15, 20, 25, 30, 35, 40, 45, 50, 55, 60, 65, 70, 75, 80, 85, 90, 100, 105, 110, 115, 120, 125, 130, 135, 140, 145, 150, 155, 160, 165, 170, 175, 180, 185, 190, 195, and 200, 300, 400, 500, 1000 μg/kg, mg/kg, μg/day, or mg/day or any range derivable therein. Furthermore, such doses can be administered at multiple times during a day, and/or on multiple days, weeks, or months.

Precise amounts of the therapeutic composition also depend on the judgment of the practitioner and are peculiar to each individual. Factors affecting dose include physical and clinical state of the patient, the route of administration, the intended goal of treatment (alleviation of symptoms versus cure, or other desired effect) and the potency, stability and toxicity of the particular therapeutic substance or other therapies a subject may be undergoing.

### IV. Methods of Determining Microbiome Composition

The methods may comprise or further comprise determining microbiome composition in a subject. The subject may be one that has been treated with the compositions of the disclosure or one that has not yet been treated with compositions of the disclosure. In some aspects, the methods relate to obtaining a microbiome profile. In some embodiments, obtaining a microbiome profile comprises the steps of or the ordered steps of: i) obtaining a sample obtained from a subject (e.g., a human subject), ii) isolating one or more bacterial species from the sample, iii) isolating one or more nucleic acids from at least one bacterial species, iv) sequencing the isolated nucleic acids, and v) comparing the sequenced nucleic acids to a reference nucleic acid sequence. When performing the methods necessitating genotyping, any genotyping assay can be used. For example, this can be done by sequencing the 16S or the 23S ribosomal subunit or by metagenomics shotgun sequencing associated with metatranscriptomics.

Methods for determining microbiome composition may include one or more microbiology methods such as sequencing, next generation sequencing, wester blotting, comparative genomic hybridization, PCR, ELISA, etc.

### V. Kits

Certain aspects of the disclosure also encompass kits for performing the methods of the disclosure, such as detection of, diagnosis of, or treatment of certain conditions and/or detection and qualitative or quantitative characterization of microorganisms. Such kits can be prepared from readily available materials and reagents. For example, such kits can comprise any one or more of the following materials: enzymes, reaction tubes, buffers, detergent, primers, probes, antibodies. In a preferred embodiment these kits include the needed apparatus for performing DNA and RNA extraction, PCR, RT-PCR, and gel electrophoresis. Instructions for performing the assays can also be included in the kits.

In a particular aspect, these kits may comprise a plurality of agents for assessing or identifying microorganisms, wherein the kit is housed in a container. The kits may further comprise instructions for using the kit for assessing sequences, means for converting and/or analyzing sequence data to generate prognosis. The agents in the kit for measuring biomarker expression may comprise a plurality of PCR probes and/or primers for qRT-PCR and/or a plurality of antibody or fragments thereof for assessing expression of the biomarkers. In another embodiment, the agents in the kit for measuring biomarker expression may comprise an array of polynucleotides complementary to the mRNAs of the biomarkers of the invention. Possible means for converting the expression data into expression values and for analyzing the expression values to generate scores that predict survival or prognosis may be also included.

Kits may comprise a container with a label. Suitable containers include, for example, bottles, vials, and test tubes. The containers may be formed from a variety of materials such as glass or plastic. The container may hold a composition which includes a probe that is useful for prognostic or non-prognostic applications, such as described above. The label on the container may indicate that the composition is used for a specific prognostic or non-prognostic application, and may also indicate directions for either in vivo or in vitro use, such as those described above. The kit may comprise the container described above and one or more other containers comprising materials desirable from a commercial and user standpoint, including buffers, diluents, filters, needles, syringes, and package inserts with instructions for use.

Further kit embodiments relate to kits comprising the therapeutic compositions of the disclosure. The kits may be useful in the treatment methods of the disclosure and comprise instructions for use.

### VI. Sequences

| | | SEQ |
|---|---|---|
| | | ID |
| Description | Sequence | NO: |
| Phocaeicola | MNETELVERCGKGDNLARKQLYERYAGQLMAVCVRYTGD | 1 |
| vulgatus | REVAQDVLHDGFLNIFRSFSQFTYKGEGSLKAWLTRIMVNE | |
| CL10T00C06 | ALGYLRKKASTNQEVIVEELPDVIDGDEDDFEQIPQSVLMQF | |
| sigma factor | IKELPDGYRTVFNLYVLEEKGHKEIAEMLGITEHTSSSQLYR | |
| | AKTLLMKKINDYRKRI | |
| Phocaeicola | MKEKDKWIKNFRTRMEGYSEPAPADLWEQLEKELDTVPKV | 2 |
| vulgatus | IPMWRRWQAVAAVALLVVVSSLTVWFWQSPSANYLEKQS | |
| CL10T00C06 - | AELNVMHESDELAPGSITPEQPMALVVPAARSGKKQHVKV | |
| anti-sigma factor | GGVAKAEALAAEQEVLLNKEVEEIQIEENYIEEQKEESVIVE | |
| | QKQQKQAGRSSYSAAKTNYAYVAPHRKKDRNWTVGLSTG | |
| | NGTFSSSTSMDGYLPLPNGTRNATMNSAYGVETRAEIDKLV | |
| | QFNNLSEGKDAQSDIKYRMPVTFGASLRFDLSDDWAVETG | |
| | VTYTQLSSETRSGTEKNNYGWEEKLHYVGIPLKVNRNIWSN | |
| | KRFEVYASAGGAVEKCVSGKRSIIGSVSTSNAGKDEQFSGG | |
| | EENVKVKPLQWSLSAAAGAQFKITEKLGIYAEPGVVYYFDD | |
| | GSNVNTIRKEHPFNFNIQLGVRFTLPK | |
| Phocaeicola | atgaaagaaaaagataaatggataaaaaatttccgcacccggatggagggctattcggaaccg | 3 |
| vulgatus | gctcctgctgatctttgggaacagttggaaaaggaactggatacggttccgaaagtaattccgat | |
| CL10T00C06 - | gtggcgtcgctggcaggctgtagcagctgttgccttgttagtggttgtgtcttcattgaccgtatg | |
| anti-sigma factor | gttttggcagtcaccttctgcaaactatttggagaaacagtcggcagagttgaatgtaatgcacg | |
| | aatccgatgagttggcgccgggatcaatcactcctgagcaacctatggccttggtagttcctgct | |
| | gcccgctctgggaaaaaacaacatgtaaaagttggtggggttgctaaggcagaggcattggct | |
| | gccgaacaggaggttttgctaaataaagaggtggaagagatacaaatagaggaaaattatattg | |
| | aagagcagaaagaggaatctgttatagtggaacaaaaacagcagaaacaggccggcagatct | |
| | tcctattcggctgccaaaacgaattacgcttacgtggctcctcatagaaagaaggacagaaact | |
| | ggacggtaggactttctaccggtaatggaaccttttcatcttcgactagtatggatggatatttgcc | |
| | tttgcctaatggcacaaggaatgcaacaatgaatagcgcttatggagtggaaacaagggctga | |
| | aattgataagctggttcagttcaataacttgtcggaaggaaaggatgcgcagtctgatataaaata | |
| | tcgtatgcctgttacttttggagcttcccttcgttttgatttaagtgatgattgggctgtagaaaccgg | |
| | ggtgacttatacccagctttcatccgaaacacgttcgggaaccgaaaaaaacaattatggttggg | |
| | aagagaaactgcattatgtgggtattcctctgaaagtgaaccggaatatatggagtaacaagcg | |
| | gtttgaagtgtatgcctcggcaggtggagcggtagaaaaatgtgtgtctggaaagcggagcatt | |
| | atcggcagtgtttctacatcaaatgcaggaaaagatgaacagttttcgggaggggaggaaaatg | |
| | tcaaagtgaaacctttgcaatggtctttgtctgcagcggcaggtgcgcagtttaagattacggag | |
| | aaattgggtatttatgcggagccgggagttgtttattactttgatgacggcagtaatgtaaatacga | |
| | ttcgcaaggaacatccttttaattttaatatacagttgggtgtgcggtttactttgccgaaataa | |
| Bacteroides | MKDEKMKDREMWMDKLKEKLENYSEPIPPFGWEQLEKEL | 4 |
| salyersiae | VPPVKKRILYPYRRWGAAAAAVLLVVASSLSIYFLNTPTAD | |
| CL02T12C01 | EIRHTVAPVLASDPDLLPPAHDPDMQVAKVEPVQPRPVIAQ | |
| (HMPREF1071_ | ARKVTERQGIEPSVPAETVTCPVEEGVNPIREAEAKETLPDE | |
| 02299) anti- | TKETKEESGTVVRPHQRSGQDKLHIPAEKPKAKKGRWSVG | |
| sigma | AAVGNGGGTSFSGNGAVEPLHSSFSDQRLNLAPTAGNEIIQI | |
| | PNNQVVVFKEGVPYLKRTDEIVDIKHHQPVSFGLSVRKGLA | |
| | KGFSVETGVTYTLLSSDVQTVVSDARIDQKLHYIGIPVRAN | |
| | WNFFDKDRFTLYVAAGGMVEKCVYGKLGNDKLTVNPLQF | |
| | SVSGAVGAQFNATKHIGVYVEPGVAYFFDDGSDVQTIRKDT | |
| | PFNFNLQAGIRFTY | |
| Bacteroides | MKERMEDELWLKKIKDKLEDYSEPLPVAGWERLEKELSVS | 5 |
| uniformis ATCC | GAPITGPHRMIPFRRWAVAAAAVLLVAVSSVSLWLLQSPVG | |
| 8492 | NEMRHTSVPALAVAPDVLPEQTVPAIRTNSIEPAYRAHGNA | |
| (BACUNI_04532) | SAPNKEASHPLVAQHIRISVGEEQQEEMLPVETADEVVGIGQ | |
| anti-sigma | QAEEPVVEDTDHETTMQTEEPETREDRYRPSGRDKLHLPEK | |
| | NSSGRDAKGWAVGLSVGNTGGFSLANEGEANVMSDYMPG | |
| | SPIYGGNVDLSSTANGIVTIPDGQELVFKDGMPYLQRREKKI | |
| | ADIDHKQPLSFGVSVRKNLAKGFSVESGLTYTYLASDVRYE | |
| | GSSEKISQKLHYIGIPVRANWNFVDAKNFTMYVSAGGAIEK | |
| | CVYGKIGTESETVKPVQLSVMGAVGAQYNISNRVGLYVEP | |
| | GVSYFFDDGSSIQTIRKENPCNFTLQAGIRLTY | |
| Parabacteroides | MSNKEMKDKERDIFDDLFRSKLQDFEADTMPGDWEAIADR | 6 |
| merdae ATCC | LPVKKSVPFRRTLRYWGAAAVISLLVVTGGVYVFNRDREH | |
| 43184 | LPIAETIRKETEAVENRLTERSESVTPAVVASKPVDRMAKRA | |
| (PARMER_03597) | VAQVTAVTTSRSDITVDEVVLSPEEDCDTVVVISSKISEASEA | |
| anti-sigma | IAVTETATLTGTHFLFADAAPVKTEEKKVRAKRWSFGMGG | |
| | GSVSAGTSNSLNAYALKNTLMTDQELLQLNSPYFNSQSPKT | |
| | NIRHKTPVSIGLGVSYSINDRFSLQSGLNYTFLSSEWETGAIY | |
| | HGETKQKLHFIGIPLSLSYKIAEWKRIQFYAAAGAMTEINVA | |
| | GKLNAKIFVENNEIRHEKKHIRMKEWMWSVNARAGASYPL | |
| | LRFLSVYAEVGAGYYFDNGSDIETIRSEKPFNVNLQAGFRFG | |
| | F | |
| Prevotella copri | MSNDWTNKLRDQLADYQEPVSHDMWAEIEQSLAQSKKVG | 7 |
| DSM 18205 | DEETDVKKTPQARYVVLKRWSVAAAIALLGVGGSYVFLHD | |
| (PREVCOP_06006) | DEPGQANLAVQSSFSTASGTSRTSSAVRAEAQSAGSKNAILL | |
| anti-sigma | AENNSSAHQVNRHSSGRQLNYSKSSVSFQERTVAADNEVM | |
| | LAAEVEMPEQSVNAEVNGRELSVLQKQNEDSERQMAVSSN | |
| | HATKPLEADGGQKLYAGRAQDGHFERSYKDKYEQNWTMN | |
| | LYAENVSLGSGSDGMSNGMYASSDPLAGGGFADHGVFLAA | |
| | ASPLRYAIPKYVEAKHHAPLAIGAQVGIGLAPRLSLSTGVVY | |
| | TRVASDFKSYGVSEFDTHQVLHYVGVPLGLNYEVWSTGGF | |
| | HAYVMAGGEADFNVKNDTKVSGHKEDVKRDGVQFSGKAS | |
| | LGAQYDVTPQVGFYIEPGAKYYFDNGSEIENTFKDKKWNFN | |
| | LQFGLRIHLK | |
| Bacteroides | MEEKELWMNKLKEKLEDYSEPIPASGWEQLEKELMPPVER | 8 |
| caccae ATCC | KIYPYRKWMMAAAAVILLALVSSVSLYFLGTPAADEIRHTQ | |
| 43185 dup2 | TPALASVLDALPAVQQPDAQGATADPVSRPMLKTDRLAKT | |
| (C5Z00_10740) | EHNISEQNTYTDQPVIQNEDEFPATDDKIDNGEKEETKLVKD | |
| anti-sigma | ANAGQKKQALETEEPRNNRPRRPSSRDKYHIPTEKKSSQKG | |
| | TWSMGFAVGNSGGASTEVGGGTQYLSRVSMAAVSNGLMN | |
| | IPEDKTLVFEDGIPYLRQAKQVIDIKHHQPISVGVSVRKSLGK | |
| | GFSVESGLTYTLLSSDVKLADGNQEVEQKLHYVGIPLRANW | |
| | NFLDKKLVTLYVSGGGMIEKCVYGKLGSEKETVKPLQFSVS | |
| | GAVGVQLNATKRIGIYMEPGVAYFFDDGSSVQTIRKENPFN | |
| | FNIQAGIRFTY | |
| Phocaeicola | MRKYKATVIVIIMVMLIPFMQSCLDDYDDDIYDMQAGMPF | 9 |
| vulgatus | NAALATVVTPSEIPGEAMIESDNDGVAYVVNPDKLTRFETN | |
| CL10T00C06 | NPGQRIFYTYINADNPTGDASKKGPFISIDYLQKILTKRMDTL | |
| (NigD) | KENEEDIYGHDGINLITPIMGKTHLTLMFQILGFNSNIKHRIS | |
| M0N98_03762 | LVATEGTVPDANGYMAVELRHNAEGDRQEYPSSGYVSFPL | |
| | LDVPGYKEGKLQGFKIKMNTINNGEETVTVSYNKSKSTNFP | |
| | FIQKGISNTKTK | |
| Phocaeicola | MKKLNLLLLAFLVVMGVTLQSCDDSDGYSLGDVAVDWAT | 10 |
| vulgatus | VNVKGAHVYDFTGDRWGQIWPATTDCFWYSPIDGQRVILY | |
| CL10T00C06 | FNPLYDNYPEGYDCSVKVLNIKEILTKPIEELTAENEEAFGN | |
| (NigD) | DPVDIFEGNMWISGGYLNIIFNQNMPSKMKHLVSLVKNTTIT | |
| M0N98_02415 | PVQDGYIHLEYRYNTYADTTGYWRNGAVSFNLKSLKITSET | |
| | KGIKVKINSAKNGEKEVSFDLKETPSPVGLSQMDFSQMEIK | |
| Phocaeicola | MKKFFGFNKLLASVAVLASVMIFQSCLDDDDNDYYRYALP | 11 |
| vulgatus | NALVTVKPVADNSFFMQLDDSTTLLPVNMKNSPYGEKEVR | |
| CL10T00C06 | ALVNFDPVNESSGEYDKAVHINWIDSILTKPIAGDLGDKNDE | |
| (NigD) | VYGTDPVDMINDWVTIAEDGYLTLRFRTMWGNRSQAHFV | |
| M0N98_03293 | NLLMSKDAENPYEVEFRHNAFGDVYGEPADGLVAFNLDSL | |
| | PDTEGKTVKLKLKWKSFNGEKTAEFDYCTRKSTPANSAAIT | |
| | AGRSVLRLK | |
| Phocaeicola | MRKKILFIIPYIPYPLDSGGNQAFFNMVEYLRHKMAVSVLLY | 12 |
| vulgatus | PETGRRMQDVESLKKIWDNVDFFIYTPKTKTVRLPKIKHPFY | |
| CL10T00C06 | YKWLQKIHASVTRKMNRQQIWTDETGEVVDLVRGKSTLFS | |
| M0N98_00432 | SCFQPLDQGYVEYVSQVAHSGFDIIQVEFYELLSLGFLLPEN | |
| | VQTIFVHHELRYIRNEKEITLFREQTAGDRMLFHIAKDFERS | |
| | ALLKYKDVIVLTEVDRKIMEDFIGRKDHIYTSPAVVQVGDR | |
| | LEQPFVPVQSGRLTFVGSEDHFPNLDAVAWFCHEVIPHLRK | |
| | RHFSFTLQVIGKWRGECINRLQSEYPELKLAGYVEDLGSFLK | |
| | GSVAVVPIRIGSGMRMKILDAVLSKVPFVTTAKGVEGIDFK | |
| | DGEDCLIVDDPAGFAEAVIALSSNPQLQRQLVTHAEDSLRQ | |
| | VYNPGQMQERRLAVYEQILGDKVG | |
| Phocaeicola | MLKQFFSLLKRYILPYRKYLTWALILNFLSQWLNVFSFMAIV | 13 |
| vulgatus | PILNILFKIDTKSYEYIPMDIHNLDKDVLINNAYYFVSNFVAT | |
| CL10T00C06 | NGAFYTLAMMGGILIFMTMLKTAGYFASAAVMVPLRTGIV | |
| M0N98_00431 | RDIRIQVYNKVLSLPLSFFSEERKGDIIARMSADVTVVENSLT | |
| | SSIDMLIRNPIALLVCFVTLFSVSWQMTLFVIFILPLTGWIMG | |
| | VVSRKLKRQSSTAQAQWGDIMSQLDETLGGLRVIKAFIAES | |
| | KMSARFSKTNNDFRDAMNEMIIRQSSAHPMSEFLGTCVIVT | |
| | VLLFGGALILNTNYAPMDAATFIFYLIILYSIINPLKEFSRAFY | |
| | NIPQGLASMERIDMILKAENHIVEPEQPLPLDAFTDKLEFKN | |
| | VSFSYVEGRPVLNHINLTVPKGKTIALVGQSGSGKSTLVDLV | |
| | PRYHDVSEGALLIDGKNVKDVSIHSLRSLIGNVNQEAILFND | |
| | TFYNNITFGVENATMEQVIEAAKIANAHDFIMETEKGYDTM | |
| | IGDRGGRLSGGQRQRVSIARAILKNPPILILDEATSALDTESE | |
| | RLVQEALERLMKSRTTIAIAHRLSTIKNADEICVLYEGDIVER | |
| | GTHDELIALNGYYKKLNDMQSL | |
| Phocaeicola | MEQIKKLAIVIPAYKGRFLKETLDSIAVQAHKDEFVLYIGDD | 14 |
| vulgatus | ASPERLDKIVESYQNKVNLVYHRFSENMGGKDLVAHWERC | |
| CL10T00C06 | IQLSAEPFIWLFSDDDLMPADGVERVMEVLSRPHHQRGYFF | |
| M0N98_00430 | RFPLAVIDGENKRIRANRPLEEGSVSCYRLLLDKLQGKIDSA | |
| | AVEYVFSREIWQSAGGFVHFPMAWCSDDATWAAFARHAG | |
| | GVISLPGQPVCWRNVEGANISNSAGHDKDKLHATILFLRWM | |
| | RNMFSDYVDDPELISALQCYIHTILRISLHKHYNICGLWGVS | |
| | MALGRFNKRAAFTTFFRNFRLFS | |
| Phocaeicola | MKILLVEDDANLREVTQRSLEKERYVVEIAADYRTALQKIE | 15 |
| vulgatus | DYDYDCILLDIMLPDGSGLDLLEKLKEMHKRENVIILSAKDS | |
| CL10T00C06 | IEDKVLGLELGADDYLPKPFHLVELNARIKSVIRRHQQGGEN | |
| M0N98_02416 | SIKYGNIEIFPDKYGVTVAGKKLELNRKEYDILVYFMNRPGR | |
| | LVNKSTLAESVWGDHIDQVDNFDFIYAQIKNLRKRLKEAGA | |
| | TPELKAVYGFGYKMVVE | |
| Phocaeicola | MKLIYYIIIRISLVLSVLLTGWAILFYFAVMDEVNDEVDDSLE | 16 |
| vulgatus | DYSEIIIIRALAGEELPSKNTASNNQYFLREVTKEYAGSCDDII | |
| CL10T00C06 | YKDSMVYIPEKDETEPARILTTIFKDDGEKFFELTVATPSIEK | |
| M0N98_02417 | EDLKDAMAGWIIFLYIALLLTIIVINVWVFYRNMRPLYVLLH | |
| | WLDKYRIGKVNEPLQNNTRVSEFRKLNEAAVRYAERSEQM | |
| | FEQQKQFIGNASHEMQTPLAICRNRLEMLMEDENLSESQLE | |
| | ELMKTHQTLEHITKLNKSLLLLSKIENGQFTDTVQVEVNKLL | |
| | RQYLEDYKEVYQYREIITSIEEEGVFYLTINETLAVVLLTNLL | |
| | KNAFVHNMDGGHIRIVITPHSVMFCNTGAAQPLDARRIFER | |
| | FYQGKKKEGSTGLGLAIADTICKMQALRLCYEYKSGEHCFT | |
| | LYASTHNQ | |
| Bacteroides | MEEQELTELCRQGDNRARKELYEQYAGRMLGICLRYAGDR | 95 |
| salyersiae | ETAQDLLHDGFLKIFGSFDKFTWRGEGSLRAWMERVMVNV | |
| CL02T12C01 | ALQYLRKNDVMSQSMALEDAPETYEEPDASEVEAIPQQVL | |
| (HMPREF1071_ | MHFVEELPAGYRTVFNLYTFEDKSHKEIAQLLGINEKSSASQ | |
| 02298) sigma | LFRAKTVLARKVKEWLMMNDR | |
| factor | | |
| Bacteroides | MEEQELAERCRQGDNLARKELYERYAGRMLSVCLRYAGD | 96 |
| uniformis ATCC | RETAQDLMHDGFLKLFDSFDKFTWRGEGSLRAWMERVMV | |
| 8492 | NTVLQYLRKNDVMNOSSALENAPEAYEEPEGSSIDVIPQKV | |
| (BACUNI_04533) | LMQFISELPAGYRTVFNLYIFEEKSHKEIARLLGINEKSSASQ | |
| sigma factor | LTRAKATLAAKVREWMKRND | |
| Parabacteroides | MTEKQLIEGCRNGERLAQKELYETYSRKMMGVCLRYVSDR | 97 |
| merdae ATCC | ETARDLLQDGFVKVFTSMDSYSGLGSFEGWMRKIFVNCAL | |
| 43184 | EYLRKSDVLREAVDLDNTVELVHPDSSAISDMSAVELMKLV | |
| (PARMER_03596) | QELPAGFRTVFNLFAIEGYSHKEISEMMNITESTSRSQFTRAK | |
| sigma factor | QLLQRRIHELY | |
| Prevotella copri | MKLESEKKLLDDINSGSRAAMHRLYERYVGYAMAVALRY | 98 |
| DSM 18205 | VPMRDDAEDVVQDSFIKVFSGISKFEYRGEGALQAWLLRIV | |
| (PREVCOP_06007) | TNEAVNFVRQQKRFTIVDEVPDDIEDEEPEVERVPPAELTRM | |
| sigma factor | IGELPDGYRLVLNMFVFEQKSHKEIAQLLGIKESSSASQYLR | |
| | AKKLLGKKVKDYLSQADCSDIKELNHINEKSNE | |
| Bacteroides | MEELELSEQCRLGNNQARKELYEQYAGRMLGICLRYTGDR | 99 |
| caccae ATCC | DTAQDLLHDGFIKIFDSFDKFTWRGEGSLRAWMERVIVNTA | |
| 43185 dup2 | LQYLRKNDVMNQSAPLEELPEKYEEPDVSDVEAIPQRVLMQ | |
| (C5Z00_10745) | FIEELPAGYRTVFNLYTFEDKSHKEIAQVLGINEKSSASQLFR | |
| sigma factor | AKSVLAKRVKEWIMNNG | |

### VII. Examples

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1—A Proteolytically-Activated Antimicrobial Toxin Encoded on a Mobile Plasmid of Bacteroidales Induces a Protective Response

### A. Introduction

Here, the inventors identify and characterize an antibacterial toxin from P. vulgatus and P. dorei whose primary structure is unrelated to any characterized protein. They show the toxin, designated BcpT, is encoded on a mobile plasmid, requires cleavage at two sites to activate its antibacterial activity, and utilizes the Lipid A-core glycan as its receptor. Exposure of sensitive cells to BcpT induces activation of a sigma/antisigma factor pair whose regulon includes genes involved in the synthesis of a protective response to two antibacterial toxins, BcpT and BSAP-3. BcpT defines a previously unidentified family of antibacterial toxins that is widespread in human gut microbiomes.

### B. Results

1. Inhibitory Activity of P. vulgatus and P. dorei Strains

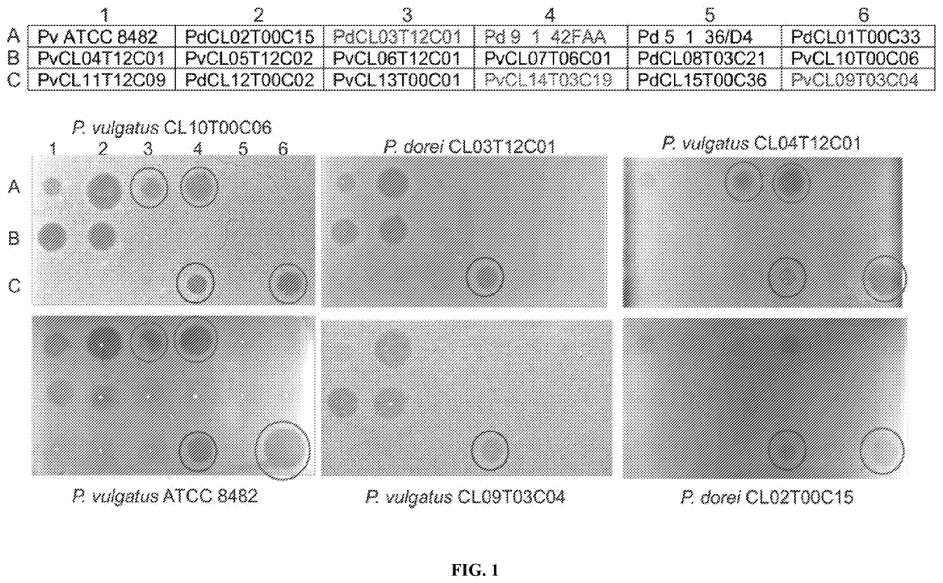

To analyze the breadth of antimicrobial toxins produced by P. vulgatus and P. dorei strains, the inventors tested 18 strains (FIG. 1, chart) for production of secreted molecules that inhibit the growth of other P. vulgatus and P. dorei strains using an agar spot overlay assay (13). Three of these strains are known to produce BSAP-3 (FIG. 1, spots A3, A4, C6) (3) and one produces bacteroidetocin A (5) (FIG. 1, C4). P. vulgatus CL10T00C06 (PvCL10) and P. vulgatus ATCC 8482 (Pv8482) are known to be sensitive to BSAP-3 and bacteroidetocin A. Here the inventors identified three other strains that produce molecule(s) that inhibit their growth (FIG. 1, left panels) (spots A2, B1, B2), as well as a faint zone of growth inhibition by a molecule produced by Pv8482 (A1). The middle panels of FIG. 1 show sensitivity of two BSAP-3 producing strains, PdCL03T12C01 and PvCL09T03C04 (PvCL09) to molecules produced by this panel of strains. These strains are resistant to their own BSAP-3 toxin as they have an altered receptor (3). However, both of these strains are sensitive to molecules produced by the strains that targeted PvCL10 and Pv8482 (A2, B1, B2), and are also sensitive to bacteroidetocin A (spot C4). The right-most panels show overlays using PvCL04T12C01 (PvCL04) and P. dorei CL02T00C15 (PdCL02) (spots B1, A2), two of the strains shown here to produce an uncharacterized toxin(s). PvCL04 and PdCL02 are relatively resistant to their own toxin(s) and appear to be less sensitive to BSAP-3 and bacteroidetocin A than strain PvCL10.

### 2. Identification of an Antibacterial Toxin

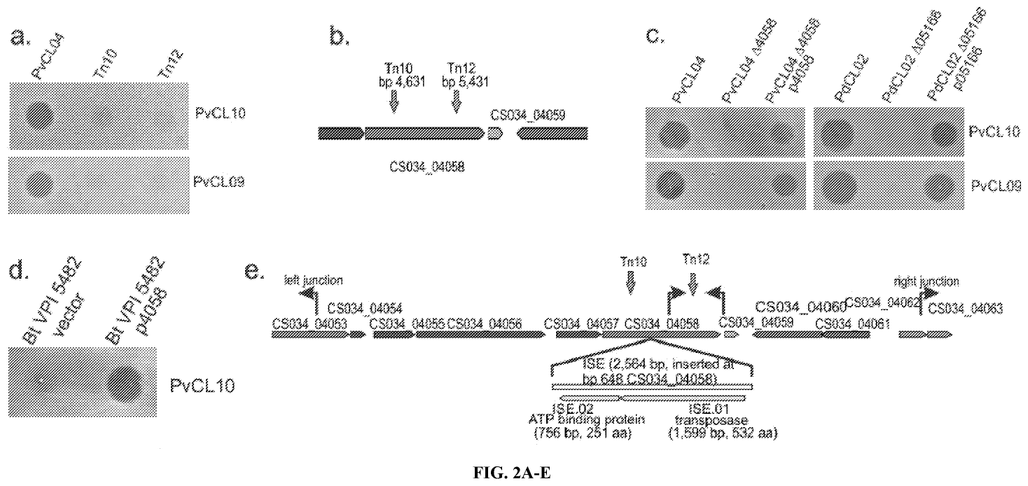

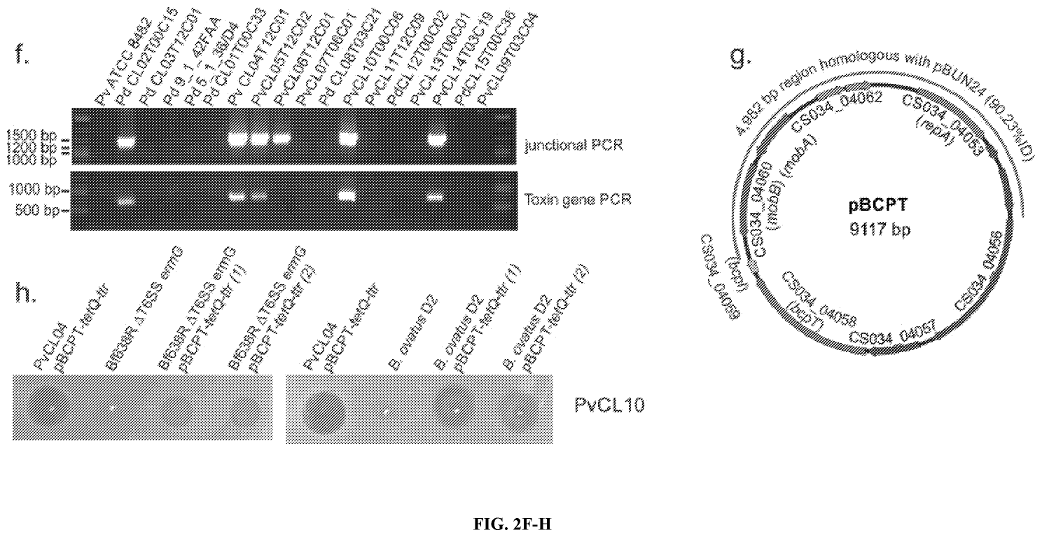

The genome sequence of PdCL02 is publicly available (GenBank accession JH724123), and here the inventors sequenced the genomes of PvCL04 and PvCL05T12C02 (PvCL05) to study the genetic basis of toxin production by these strains. The inventors screened a transposon bank of strain PvCL04 for loss of toxin activity and identified two transposon mutants that lost the ability to inhibit the growth of strains PvCL10 and PvCL09 using the agar overlay assay (FIG. 2a). Both transposon insertions interrupted the same gene (CS034_04058) (FIG. 2b). The genomes of PvCL05 and PdCL02 each contain a gene that is identical to CS034_04058, as does the genome of the bacteroidetocin A producing strain PvCL14. CS034_04058 encodes a 499 amino acid protein with a predicted signal peptidase II cleavage site, indicating it is likely an outer surface lipoprotein. Mutants with internal deletions of this gene in both PvCL04 and PdCL02 (the homologous gene is designated HMPREF1063_05166 in PdCL02) do not inhibit growth of either PvCL10 or PvCL09 and toxin activity is restored when the gene is added in trans to these mutants on a plasmid (FIG. 2c). When CS034_04058 is transferred into B. thetaiotaomicron VPI5482, it confers toxin activity to this strain (FIG. 2d).

### 3. The Toxin is Encoded on a Small Mobile Plasmid

The contigs containing the toxin encoding gene in PvCL04, PvCL05, PdCL02 and PvCL14 are similar in size (9089-9140 bp), suggesting this gene is carried on a plasmid. Using PCR with two outwardly facing primers (FIG. 2e), the inventors confirmed the DNA is circular (FIG. 2f, 2g). The inventors used these primers and a second primer set (FIG. 2e) to PCR amplify the end of the toxin gene, with DNA from all 18 strains tested in FIG. 1 (eight of which do not have genome sequences). Amplicons across the contig junction are produced for all strains containing the toxin gene. PvCL06T12C01 contained a similar plasmid based on amplification across the junction, but no toxin gene was amplified (FIG. 2f) nor toxin activity detected (FIG. 1). The other unexpected finding is that the toxin gene was amplified using PvCL10 as template (FIG. 2f), however, PvCL10 does not produce this toxin and is very sensitive to it (FIG. 1). Analysis of the PvCL10 genome sequence shows that the toxin gene is interrupted at bp 638 by a 2563 bp insertion sequence disrupting the gene (FIG. 2e).

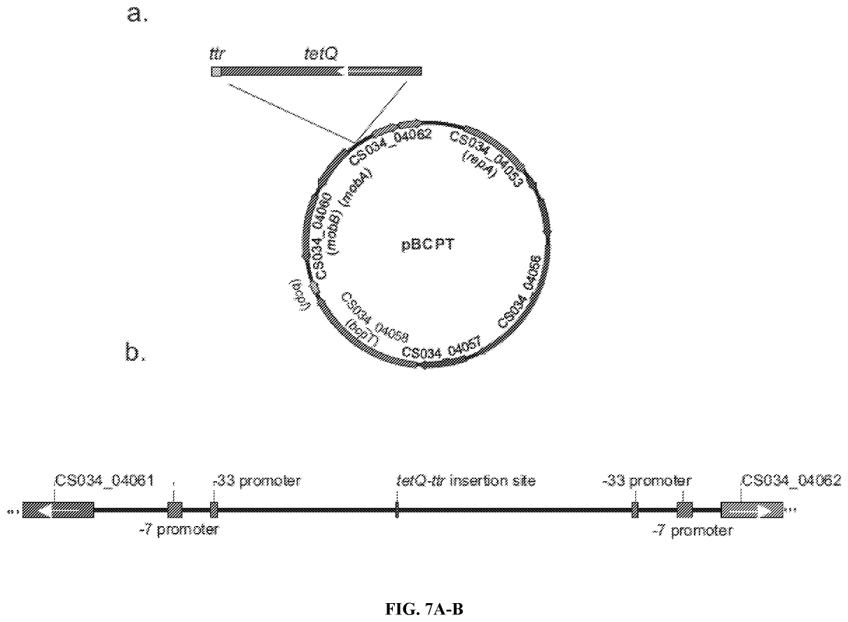

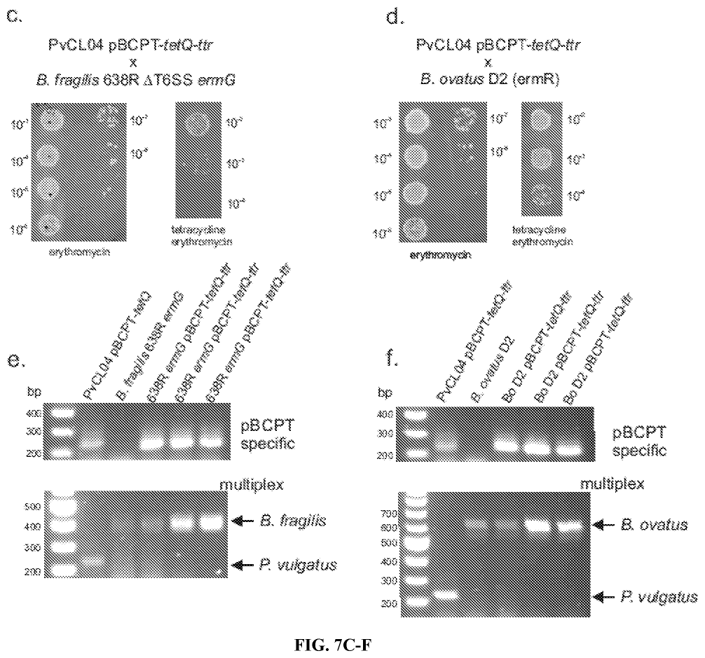

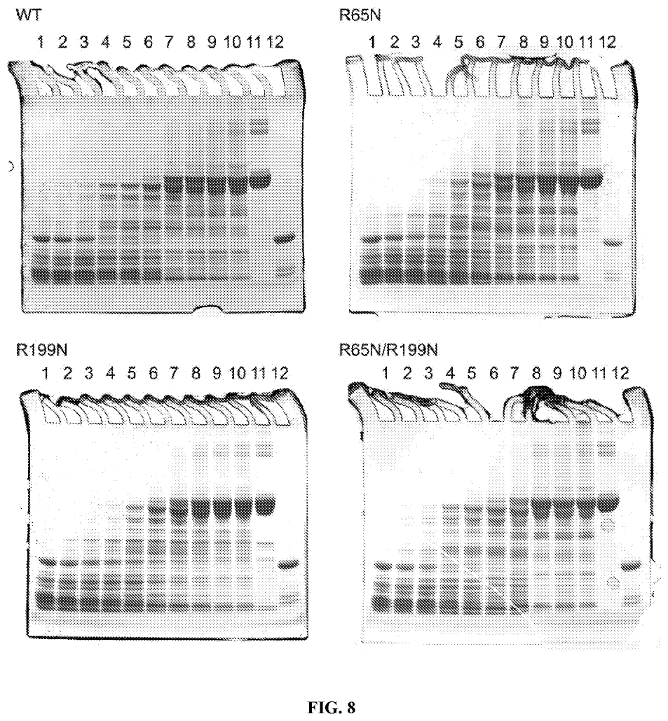

Further analysis of the sequence of this plasmid revealed that more than half of the plasmid (4982 bp) is 90.23% identical to a previously described ˜8.9 kb cryptic plasmid (pBUN24) of B. uniformis BUN24 (14), which was isolated from the feces of a six-year-old girl. Strains of P. vulgatus, Bacteroides intestinalis and Parabacteroides distasonis isolated from the same individual at age 11 also contained plasmids of identical size, two of which are 100% identical to pBUN24, suggesting intra-ecosystem conjugal transfer (14). The region of pBUN24 that aligns with the plasmid reported here is shown as a pink line of FIG. 2g and includes genes involved in replication, plasmid maintenance and mobilization (Supplementary Table 1, (14)). Four other genes including the toxin gene are not present in pBUN24. Based on this plasmid's likely mobility via conjugation, the inventors named this toxin BcpT, for Bacteroidales conjugally transferred plasmid-encoded toxin and the plasmid was named pBCPT. To confirm that pBCPT is mobile, the inventors used strain PvCL04 as the pBCPT donor and first deleted ermG from the chromosome, making the strain erythromycin sensitive. Next, the inventors added tetQ with a downstream transcriptional termination region (ttr) into pBCPT between genes CS034_04061 and 04062 (FIG. 7a, b). For transfer studies, this strain was co-cultured on plates with B. fragilis 638RΔT6SSermG, which is unable to fire its GA3 T6SS so that it cannot antagonize the donor strain, and B. ovatus D2 (naturally erythromycin resistant). Tetracycline and erythromycin resistant transconjugants were selected (FIG. 7c, d) and passaged twice and then PCR was performed, which confirmed that pBCPT was transferred to the recipient B. fragilis and B. ovatus strains (FIG. 7e, f). In addition, these two strains were able to inhibit the growth of BcpT sensitive strain PvCL10 in overlay assays, albeit to a lesser extent than the donor PvCL04 (FIG. 2h). These data confirm that pBCPT is a mobile plasmid.

### 4. BcpT Activation

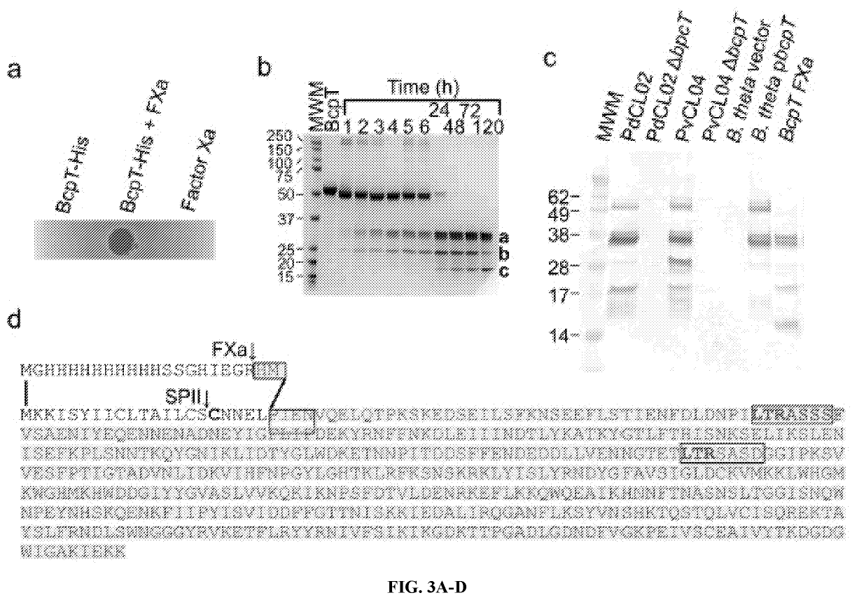

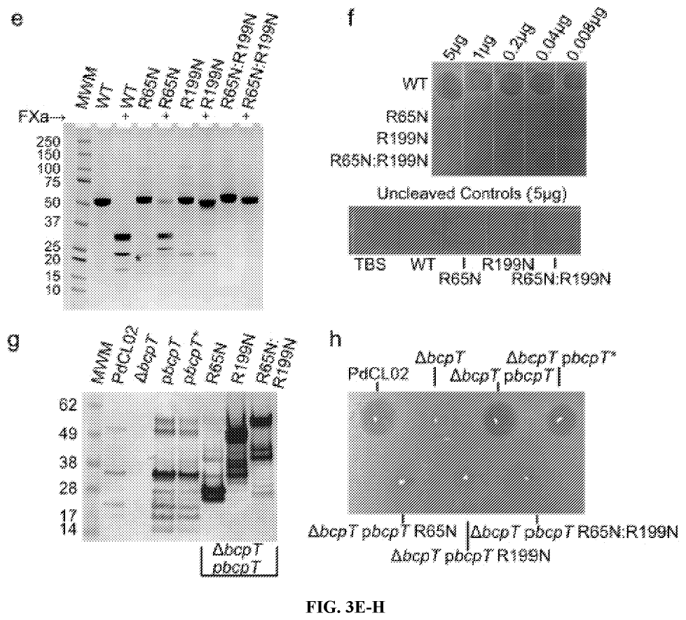

To gain a deeper understanding of the antibacterial activity of BcpT, an N-terminal His-tagged derivative was constructed. The recombinant protein purified from E. coli did not exhibit antibacterial activity when spotted onto an overlay of sensitive strain PvCL10 (FIG. 3a). Predicting that the N-terminal His-tag may inhibit activity, the inventors used Factor Xa (FXa) to remove the His-tag. This resulted in antibacterial activity (FIG. 3a), suggesting that the tag inhibited toxin activity. However, analysis of the cleavage pattern produced when the His-tagged BcpT is treated with FXa revealed it cleaves not only at the FXa specific site (I-E-G-R), but also at other sites, resulting in three fragments, not including the His-tag (FIG. 3b). Fragments a and b appear first, whereas fragment c appears upon longer incubation times and coincides with a decreased abundance of fragment b. N-terminal sequencing showed FXa cleaves after R65 and R199 of two Leu-Thr-Arg (LTR) motifs (FIG. 3d). Fragment a encompasses residues 200-499, fragment c encompasses residues 66-199, and fragment b encompasses residues 18-199. Fragment b is an intermediate cleavage product when the R65 site is uncleaved, as FXa cleaves more slowly at R65. Culture supernatants of PdCL02 and PvCL04 revealed BcpT fragments similar in size to those generated by FXa cleaved recombinant BcpT (FIG. 3c) demonstrating that BcpT cleavage occurs in the producing strains. BcpT is also present in cleaved form in the supernatant of B. thetaiotaomicron VPI-5482 when bcpT is provided in trans (FIG. 3c), demonstrating that this protease activity is not specific to toxin-producing strains. BcpT has two potential sites of glycosylation¹⁵, and therefore the migration of these fragments may be slightly altered compared to those generated from the recombinant protein.

To test whether cleavage at R65 and/or R199 is required for BcpT activation, single and double asparagine substitutions of R65 and R199 were generated, the proteins were purified and treated with FXa. BcpT^{R65N} is cleaved only at the R199 site, whereas BcpT^{R199N} and BcpT^{R65N:R199N} are not cleaved by FXa (other than releasing the His-tag) (FIG. 3e). Agar spot overlay assays using purified FXa treated His-BcpT site mutants showed that these altered BcpT proteins fail to inhibit the growth of PvCL10 (FIG. 3f), indicating cleavage at one or both sites is necessary for activity (FIG. 3f). This difference in activity was not due to aberrant folding as the chymotrypsin digestion profiles of all three mutant proteins are similar to WT at various ratios of chymotrypsin to protein (FIG. S2).

To confirm that R65 and R199 are necessary for cleavage of native BcpT in the producing strain, BcpT site mutant constructs were altered to replace the His-tag with the native signal sequence, cloned into a Bacteroidales expression vector, and transferred into PdCL02 ΔbcpT. The reconstructed wild-type construct, designated pbcpT*, is cleaved in the same manner as the original pbcpT construct, whereas the Asn site mutants at R65 or R199 result in aberrant cleavage patterns (FIG. 3g) and are inactive (FIG. 3h).

5. Identification of Proteases that Activate BcpT

The rate of BcpT cleavage by FXa is slow, typically taking 24-48 hours to fully cleave BcpT and this cleavage was serendipitous, as the LTR sequence is not a known recognition site for FXa. Fragipain (Fpn) is a C11 protease family member and is required for activation of B. fragilis enterotoxin, Bft (16), which affects epithelial cells (17). Fragipain also releases many proteins from the surface of B. fragilis (18). Fpn-like proteases are widely distributed in the Bacteroidetes phylum (18). Fpn cleaves Bft at a QTR site and based on the activation sites of BcpT (LTR), the inventors predicted an Fpn-like protease(s) cleaves and activates BcpT. Two genes of the PdCL02 genome, HMPREF1063_01439 and HMPREF1063_00451, encode proteins that are 48% and 45% similar to Fpn, respectively, and 47% similar to each other. The inventors designated these proteins doripain A (DpnA) and doripain B (DpnB), which are conserved in P. dorei and P. vulgatus strains. Both DpnA and DpnB contain SpII cleavage sites with carboxylate containing residues just after the lipidated cysteine suggesting they are transported to the surface (19), as has been shown for Fpn (18).

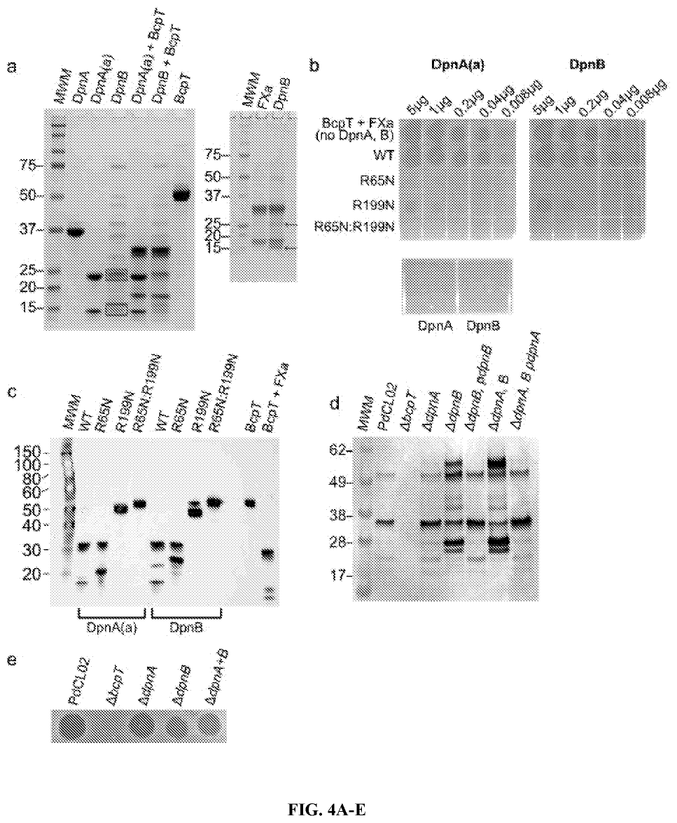

The dpnA and dpnB genes were codon optimized for expression in E. coli and cloned into pET21b removing the signal sequence and creating an N-terminal His-tagged derivative. Fpn, like most C11 proteases, contains an activation loop that occludes the active site of the protein prior to being cleaved (20). Like Fpn, recombinant DpnB is cleaved during expression and purification from E. coli, producing two fragments (FIG. 4a). In contrast, DpnA remains full-length and cannot activate BcpT (FIG. 4A) unless treated with trypsin to cleave its activation loop, hereafter referred to as DpnA(a) (FIG. 4a, 4b). The active forms of both proteases cleave and activate BcpT (FIG. 4a, 4b). Furthermore, both proteases can cleave BcpT^{R199N} at the R65 site, which FXa does not (FIG. 4c). BcpT^{R65N} cleaved with DpnA(a) or DpnB exhibits no activity and BcpT^{R199N} cleaved with these proteases only possesses slight activity (<1% WT) (FIG. 4b), therefore, full activity requires cleavage at both LTR sites.

To determine the role of DpnA and/or DpnB to the cleavage of BcpT in vivo, the inventors deleted both dpn genes individually and together in PdCL02 and analyzed BcpT fragments in the supernatants and BcpT activity in agar overlays. There are no differences in the sizes of the BcpT fragments found in the supernatant of PdCL02 ΔdpnA versus WT (FIG. 4d), but the PdCL02 ΔdpnB mutant has an altered cleavage pattern with more of the toxin in the uncleaved form. The WT cleavage pattern is restored when dpnB is restored in trans to PdCL02 ΔdpnB. Deletion of both dpn genes reveals a BcpT cleavage pattern like that of ΔdpnB (FIG. 4d), however, enough of the activated toxin is still present to inhibit the growth of sensitive strain PvCL10 (FIG. 4e). Together, these data indicate that BcpT can be cleaved by both doripains in vitro although DpnB appears to be primarily responsible for directly or indirectly cleaving BcpT in the bacteria, and partial cleavage of BcpT occurs in the absence of both proteases.

### 6. Identification of the Toxin Receptor

To identify the BcpT receptor, transposon banks were generated in PvCL10 and enriched for resistant mutants by co-culture with the bcpT⁺ strain PdCL02. The surviving tn mutants were individually screened in overlay assays. Several transposon mutants partially resistant to the toxin were identified (discussed below), however, no mutants that were completely resistant were selected in this screen, suggesting that BcpT interacts with an essential molecule.

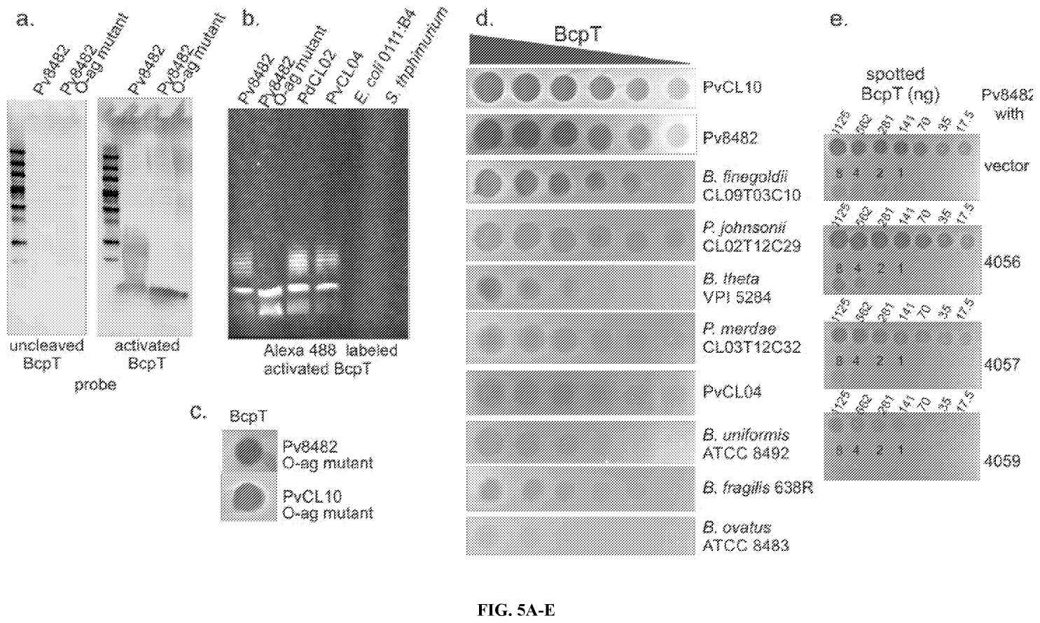

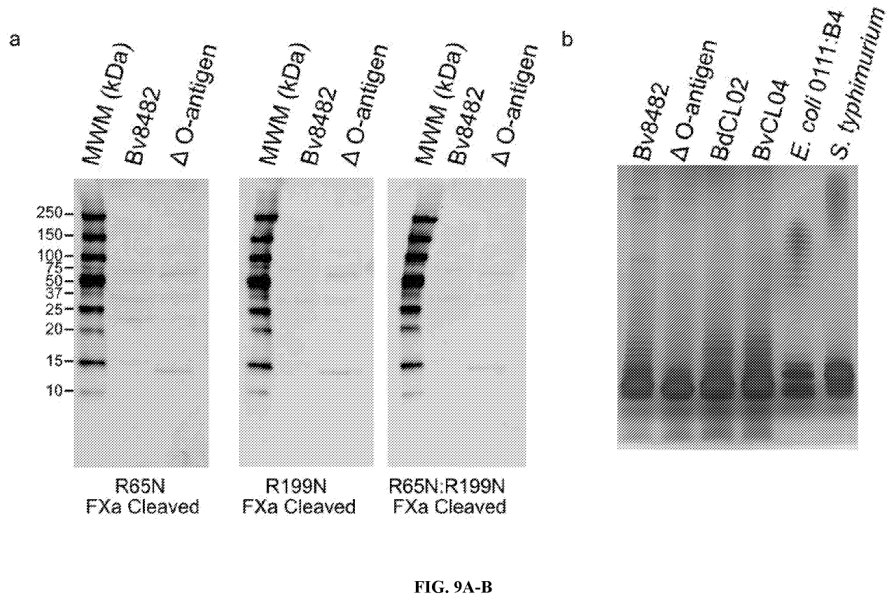

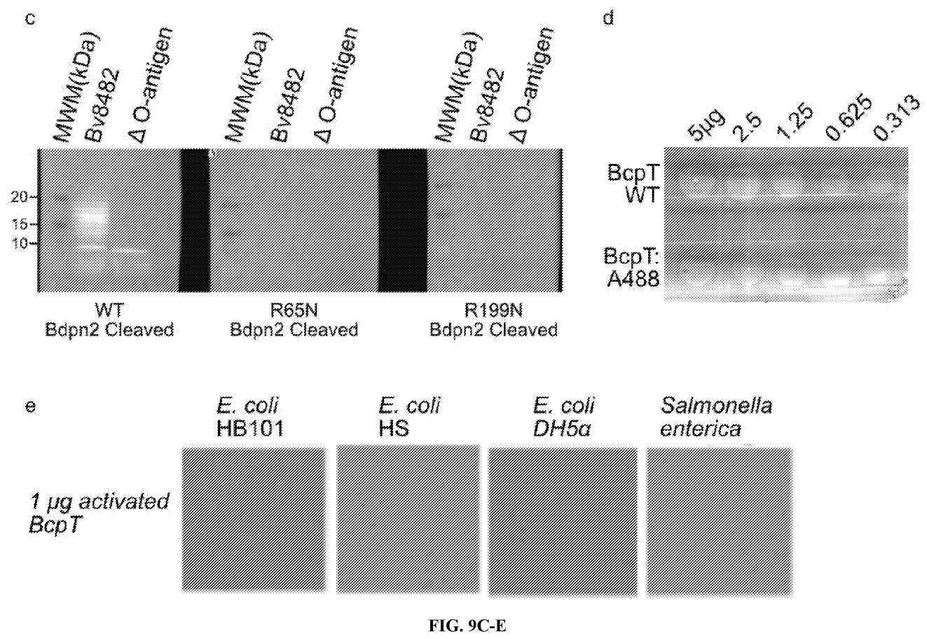

As a genetic approach failed to reveal the receptor of BcpT, the inventors used a receptor blot technique (23). The inventors probed a Western blot containing purified membranes from the sensitive Pv8482 strain and its cognate O-antigen mutant with proteolytically activated and unactivated BcpT. Binding of BcpT to membrane components was detected using affinity purified rabbit α-BcpT. Activated BcpT binds a low molecular weight molecule in the O-antigen mutant that is the size of the Lipid A-core glycan (3), and BcpT binds larger and heterogeneously sized molecules in the WT, indicative of a laddering O-antigen (FIG. 5a). Unactivated BcpT and the cleaved R65N, R199N and R65N/R199N site mutants do not exhibit any detectable binding (FIG. 5a, FIG. 9a). These data strongly suggest the receptor is the Lipid A-core glycan of the LPS and that unactivated BcpT is unable to bind. As confirmation that BcpT binds the Lipid A-core glycan, LPS and the Lipid A-core were purified from Bv8482 WT and the Bv8482 O-antigen mutant, respectively, as well as LPS from the BcpT-producing PvCL04 and PdCL02 strains (FIG. 9b). Blots containing these molecules were probed with BcpT labeled with the sulfhydryl-specific maleimide derivative of Alexa Fluor 488, which labels one or more of the 3 cysteine residues in BcpT (C270, C412 and C479) but does not affect its antibacterial activity (FIG. 9d). These analyses confirmed that activated BcpT binds the Lipid A-core glycan portion of the LPS molecule of P. vulgatus and P. dorei strains but not the LPS of E. coli or Salmonella typhimurium (FIG. 5b, FIG. 9c), which are not growth inhibited by activated BcpT (FIG. 9e). In addition, BcpT is potent against O-antigen mutants of both Pv8484 and PvCL10 (FIG. 5c). Mutations that affect portions of the core glycan are lethal in many Gram-negative bacteria, likely explaining why the genetic approach failed to identify the receptor,

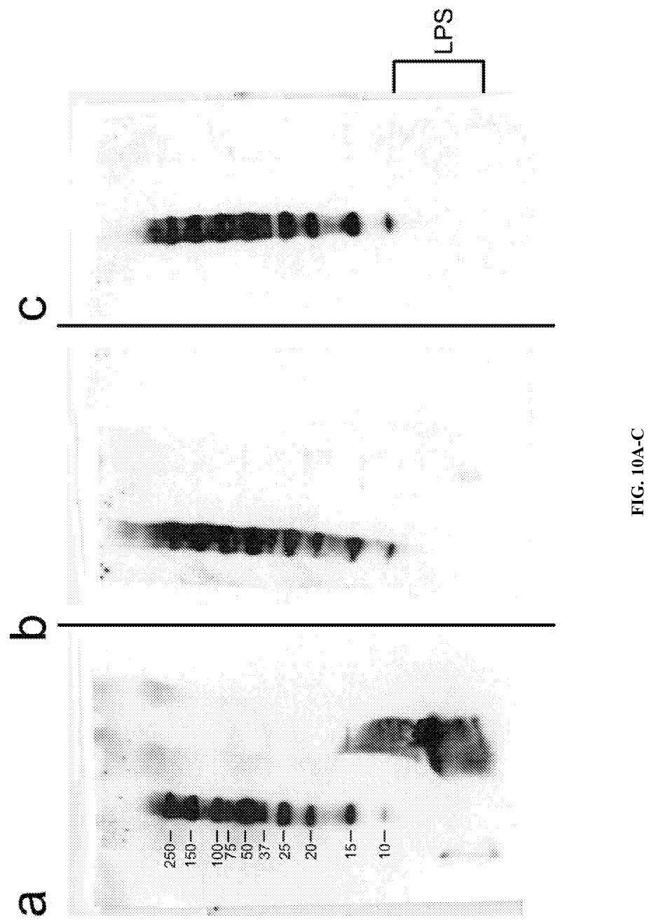

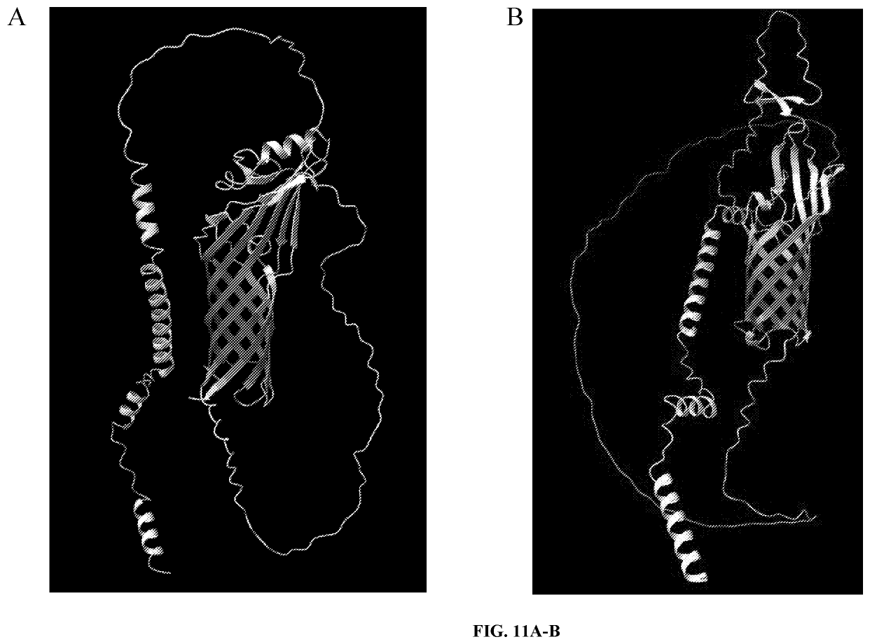

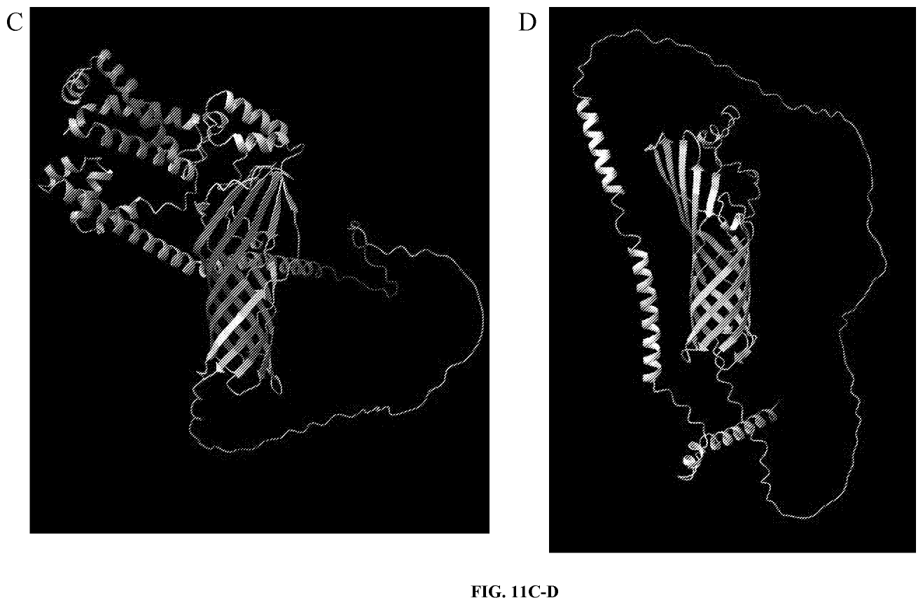

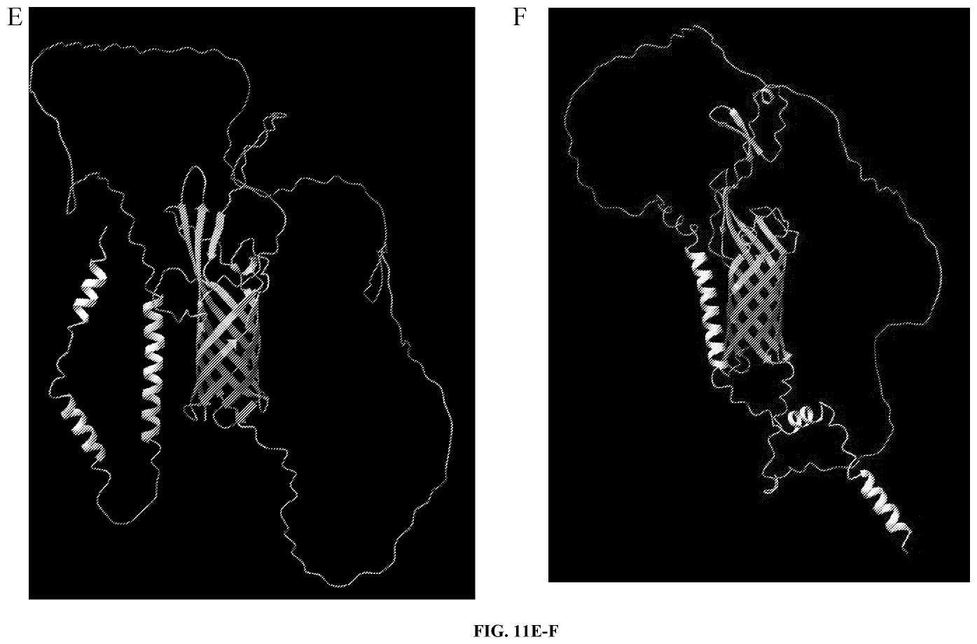

To better understand the fragments of BcpT that are involved in receptor binding, the inventors performed receptor blocking assays. The inventors found that purified recombinant amino terminal fragments 18-199 and 65-199 effectively block binding of activated BcpT to the Lipid A-core. When each fragment was mixed at a 10-molar excess to activated, purified BcpT in the blot probe buffer, incubated overnight, washed and then probed with anti-BcpT, binding of activated BcpT to the Lipid A-core was eliminated (FIG. 10). Furthermore, these blots show that neither amino terminal fragment binds to the Lipid A-core since the antibody recognizes both these fragments (FIG. 3c, g). Hence, these data show that the C-terminal 200-499 fragment binds the receptor and that the N-terminal fragment likely blocks this binding site. These data also strongly suggest that the antibacterial activity is contained within the 200-499 fragment. The inventors were unable to purify the 200-499 fragment to test its binding in the absence of the 18-199 fragment, which suggests that the amino terminal fragment is also important to the folding and/or stability of the 200-499 fragment.

As related species often share a common Lipid A-core glycan, the inventors tested the ability of purified, recombinant BcpT to inhibit the growth of seven additional Bacteroides, Phocaeicola and Parabacteroides species (FIG. 5d). BcpT sensitivity varied wherein some strains, such as B. finegoldii CL09T03C10 and P. johnsonii CL02T12C29 are inhibited at low BcpT concentrations. In contrast, B. fragilis 638R and B. thetaiotaomicron VPI-5482, are only affected by the highest BcpT concentrations and B. ovatus ATCC 8483 is the most resistant. Although the structure of the core glycan of a P. vulgatus strain has been elucidated (24), the structure of the core glycan not been determined for other strains or species, which will be necessary to determine whether the basis for the varying sensitivities is based on the structural differences. Purified BcpT also binds the LPS of the BcpT-producing strains PvCL02 and PvCL04 (FIG. 5b), yet PvCL04 is less sensitive to purified recombinant BcpT than PvCL10 and Bv8482. In addition, native BcpT produced from these strains does not potently inhibit them in overlay assays (FIG. 1), suggesting the presence of an immunity protein.

### 7. Identification of a BcpT Immunity Protein

Immunity proteins are typically encoded just downstream of the toxin gene. Of the open reading frames of the pBCPT plasmid, only three do not have obvious functions in other cellular processes CS034_4056, CS034_4057, and CS034_4059) (FIG. 2g). Strain PvCL10 also contains the pBCPT plasmid, with a 2564-bp insertion in bcpT that inactivates it (FIG. 2e) and this strain is highly sensitive to BcpT (FIG. 1). As genes CS034_4056 and CS034_4057 are upstream of the gene disruption, their expression should not be altered in strain PvCL10, however, the small gene downstream of bcpT may be transcriptionally affected by the insertion, making this the most likely immunity gene candidate. Each of these three genes was cloned into expression plasmid pFD340 and place in trans in Pv8482. Overlay assays were performed with dilutions of activated BcpT, revealing that the small 174 bp gene CS034_4059 just downstream of bcpT confers at least 8-fold protection to BcpT, whereas genes CS034_4056 and CS034_4057 confer no protection (FIG. 5e). Gene CS034_4059 encodes a protein of 57 aa (named herein BcpI), which has an SpII signal sequence suggesting it is a lipoprotein tethered to the outer membrane. BcpI lacks carboxylate containing residues just after the lipidated cysteine that would suggest it is flipped to the outer surface (19) and therefore, is likely on the periplasmic side.

### 8. BcpT Induces a Protective Stress Response

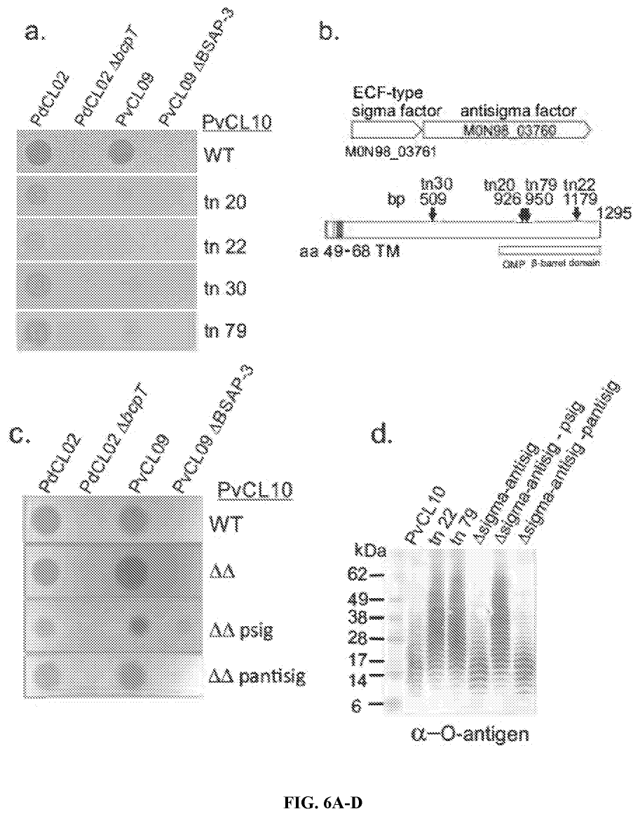

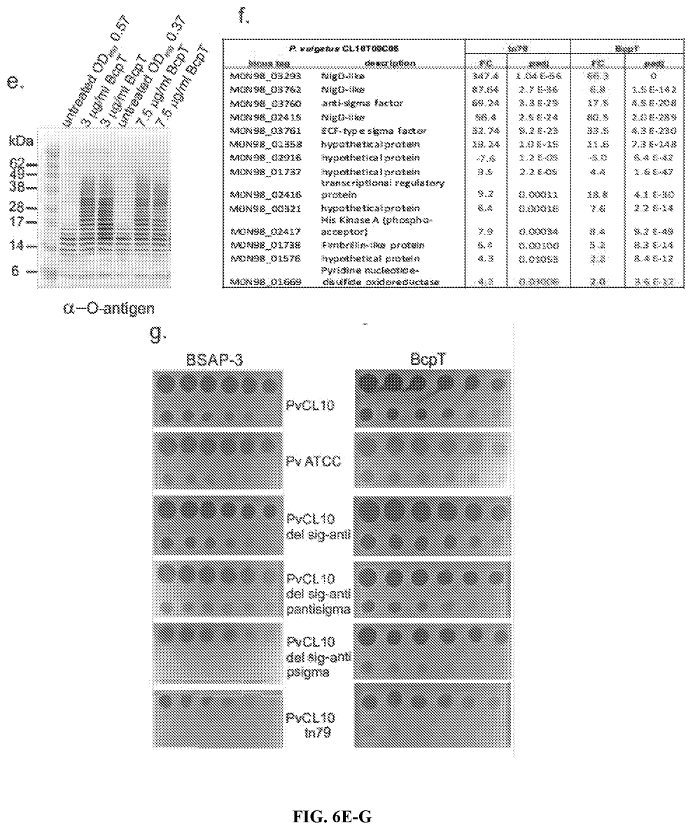

As indicated above, the PvCL10 tn mutant screen revealed four mutants partially resistant to BcpT (FIG. 6a). Unexpectedly, these mutants exhibited an increased resistance to BSAP-3 (FIG. 6a), the MACPF toxin produced by PvCL09, which uses the O-antigen of sensitive P. vulgatus and P. dorei strains as its receptor (3). These mutants all mapped to MON98_03760, a gene encoding an unusual anti-sigma factor (FIG. 6b), which is downstream of a sigma factor gene (MON98_03761). Most anti-sigma factors have cytoplasmic, transmembrane and periplasmic domains. The cytoplasmic domain sequesters the sigma factor until an extracytoplasmic signal is detected releasing the sigma factor to initiate transcription of its regulon (reviewed in (21)). The anti-sigma factor encoded by MON98_03760 is much larger and is predicted to span the periplasmic space with a β-barrel outer membrane domain at the C-terminus (FIG. 6b). A similar ECF-sigma factor/anti-sigma factor pair was described in B. fragilis and shown to respond to oxidative stress (22). Full-length orthologs of this gene pair are present in all Bacteroides/Phocaeicola species analyzed, although anti-sigma factor similarity is low between species (˜50-70% similarity). Among P. dorei and P. vulgatus strains, these proteins are greater than 98% (sigma factor) and 95% (anti-sigma factor) identical. Attempts to delete the anti-sigma factor gene from PvCL10 were not successful, but the inventors could simultaneously delete both the sigma factor and anti-sigma factor genes and then restore each gene individually in trans. The partial protection from BcpT and BSAP-3 toxicity was reproduced in strains with an active sigma factor (i.e. no functional anti-sigma factor gene), but not in strains where the anti-sigma factor is intact or when both genes are deleted (FIG. 6c). Hence, these results show that the regulon of this sigma/anti-sigma pair likely includes factors that provide partial protection to both BcpT and BSAP-3.

The receptor of BSAP-3 is the O-antigen; however, as determined above, it is not the receptor of BcpT (FIG. 5c). Strains in which the anti-sigma factor is impaired (allowing the sigma factor to transcribe its regulon) synthesize LPS molecules with substantially more O-antigen repeat units than the sensitive WT strain (FIG. 6d). These data likely explain why these mutant strains are protected from BSAP-3: longer O-antigens would place BSAP-3 too far from the membrane to insert its β-barrel pore. It is possible that longer O-antigen chains may impair BcpT access to the Lipid A-core. As the induction of the sigma/antisigma factor regulon provided partial protection to BcpT, the inventors sought to determine whether BcpT exposure induced this protective response.

The inventors first treated sensitive strain PvCL10 with 3 μg/ml or 7.5 μg/ml BcpT for 3 hours, which resulted in much longer O-antigens compared to untreated bacteria (FIG. 6e). These data suggest that BcpT induces the protective sigma factor regulon. Transcriptomic analyses were performed on untreated and BcpT-treated PvCL10 where BcpT was added at a concentration that allowed for slow, rather than arrested growth. The inventors also performed transcriptomic analysis of PvCL10 tn mutant 79, a mutant where the antisigma factor gene was interrupted, which confers partial protection to BcpT (FIG. 6a). After three hours of BcpT treatment, the MON98_03761 sigma factor gene was upregulated 33-fold (e-value of 4e-230) (Supplementary Data 1, FIG. 6f). In PvCL10 tn mutant 79, there were only 15 differentially expressed genes (DEGs) by the strict criteria used, 14 of which are upregulated compared to WT PvCL10. Importantly, all 15 of these genes are also differentially regulated in the same manner in BcpT-treated cells (Supplementary Data 1, FIG. 6f). BcpT treatment also induced numerous other genes, many of which were shown to be induced upon treatment with bacteroidetocin A (Supplementary Data 1), which binds and inhibits BamA leading to severe outer membrane defects and cell death (8). These data suggest that BcpT-treated bacteria are responding to cell envelope stress, which is likely the signal for induction of the sigma/anti-sigma regulon.

To better quantify the protective effect of the MON98_03760-61 regulon, the inventors analyzed the degree to which the various PvCL10 sigma/antisigma factor mutant strains are protected when treated with BSAP-3 or BcpT. The inventors show that the mutants in which the anti-sigma factor is interrupted (PvCL10 tn mutant 79) or missing (delta sig/anti psig) survive exposure to a 100-fold higher concentration of BSAP-3 and approximately 8-fold higher concentration of BcpT compared to WT (FIG. 6g).

9. Prevalence of bcpT in Genomes and Human Gut Metagenomes

The prevalence of bcpT and pBCPT was examined among 1148 non-redundant Bacteroidales genomes with species designations, including strains from 14 different families and 41 different genera (Supplementary Data 2). The inventors used blastn to search for the 1500-bp bcpT gene and for the 5058-bp plasmid backbone, which excludes the four genes specific to pBCPT. bcpT was present (98.8-100% identity) only in genomes of P. dorei (4/26 strains) and P. vulgatus (10/88 strains) with one exception, Parabacteroides goldsteinii 910340 isolated from a blood sample in Denmark (25). In most strains, the bcpT ortholog was contained on a contig ranging from 9.1-9.5 kb that matched the pBCPT backbone at 99-100% DNA identity. B. ovatus An161, isolated from the caecum of a chicken in China (26), was the only other Bacteroides genome that encoded a product similar to BcpT. The ortholog from this strain (B5F02_25665) is 78.4% identical to bcpT and is contained on a 9079 bp contig, but it does not align with pBCPT (Supplementary Data 2). Using blastp, the inventors found BcpT orthologs with less similarity (˜50-70% similar) encoded in three Prevotella intermedia, two Prevotella enoeca and one Prevotella shahii strains (Supplementary Data 2). These orthologs are present on large contigs, indicating they are likely not plasmid encoded. Although pBCPT is primarily restricted to P. dorei and P. vulgatus, plasmids that aligned with the backbone of pBCPT (lacking bcpT) at lower identity were identified in 15 different Bacteroidales species (Supplementary Data 2).

To determine the prevalence of bcpT in sequenced human gut metagenomes and to determine if there are geographical differences, the inventors analyzed 15 different human gut metagenomics datasets including 1767 non-longitudinal metagenomes from different geographies (Supplementary Data 3). These data show that the Japanese dataset has the highest prevalence of metagenomes with bcpT (20.7% positive), followed by a US dataset (18.5% positive). Gut metagenomics datasets from Madagascar (112 metagenomes), Ethiopia (24 adult metagenomes) and Peru (36 metagenomes), collectively have a single metagenome with bcpT. This observation is consistent with a recent study showing a higher rate of horizontal gene transfer in more industrialized populations (27). Of the 1767 human gut metagenomes analyzed, 225 contain bcpT based on the inclusion criteria. Of these 225 bcpT-positive metagenomes, analysis using MetaPhlAn (28) indicated that all but two of these metagenomes have either P. vulgatus or P. dorei present at a detectable level (Supplementary Data 3). In total, these genomic and metagenomics data indicate that pBCPT is present in numerous countries, yet absent from certain human populations, with a strong bias for P. vulgatus and P. dorei strains.

### C. Methods

### 1. Bacterial Strains and Growth Conditions

Bacterial stains used in this study are listed in Supplementary Table 3. Bacteroidales strains were grown in basal liquid medium (34) or on BHIS plates (35). Antibiotics used for selection include erythromycin (10 μg/ml), gentamicin (200 μg/ml), tetracycline (4 μg/ml) carbenicillin (100 μg/ml), and kanamycin (50 μg/ml).

### 2. Agar Spot Overlay Assay

P. vulgatus and P. dorei strains were dotted in 5 μl aliquots from broth cultures onto rectangular BHIS plates and grown overnight. Bacteria were removed using two applications of filter paper and plates were exposed to chloroform vapor for 15 minutes to kill residual bacteria (13). Strains being tested for sensitivity were grown in basal medium to an OD₆₀₀ of 0.7 and added to 6 ml top agar (BHIS 0.75% agar), which was the spread over the plate and grown anaerobically overnight. For toxin spotting assays using recombinant BcpT, strains were grown overnight, added to top agar and was poured onto BHIS plates. After hardening, dilutions of BcpT samples (5-7.5 μl) were spotted unto the top agar. Plates were incubated under anaerobic conditions at 37° C. overnight.

### 3. Transposon Mutagenesis of PvCL04 and PvCL10

Random mutagenesis of PvCL04 was performed using the transposon containing plasmid pYT646b and individual mutants were screened using the agar spot overlay assay for those that no longer inhibited the growth of PvCL10. The insertion sites of tn10 and tn12 were identified as previously described (36). Random mutagenesis of PvCL10 was performed using pSAM_BcellWH2 and the insertion sites identified by PCR as described (37).

### 4. Creation of Deletion Mutants and Complementing Clones

An internal non-polar deletion mutant of bcpT of PdCL02 (HMPREF1063_05166) was constructed by amplifying DNA upstream and downstream of the gene using the primers listed in Supplementary Table 2. PCR products were cloned by three-way ligation into pBluescript utilizing blue and white selection, and then excised using BamHI and MluI and cloned into pKnock-bla-ermGb. This plasmid was conjugally transferred from E. coli into PdCL02. Cointegrates were selected by gentamycin/erythromycin resistance. Double cross-outs were screened via PCR for mutant genotype.

Deletion of bcpT of PvCL04 (CS034_04058) was constructed by amplifying DNA upstream and downstream of the region to be deleted and cloning by three-way ligation into BamHI/MluI digested pBluescript utilizing blue and white selection. This construct was then transferred into pKnock-bla-tetQ and conjugally transferred from E. coli to PvCL04. Cointegrates were selected by gentamycin/tetracycline resistance. Double cross-out recombinants were screened via PCR for mutant genotype.

Deletion of genes dpnA (HMPREF1064_01439) and dpnB (HMPREF1064_00451) from PdCL02T12C06 was created by amplifying regions upstream and downstream of the genes and cloning using NEBuilder into BamHI-digested pLGB13 (35). The construct was transferred from E. coli into PdCL02. Cointegrates were selected on gentamycin/erythromycin and double recombination cross-outs were selected on BHIS with anhydrotetracycline (aTC, 50 ng/ml) and screened via PCR for mutant genotype.

The internal, non-polar deletion mutant of the PvCL10 sigma factor/anti-sigma factor genes (MON98_03760-61) was constructed by amplifying DNA upstream and downstream of the region to be deleted and cloned into the BamHI site of pKnock-bla-ermGb using NEBuilder, transformed into E. coli S17 λ ir, and conjugally transferred into PvCL10. Cointegrates were selected on gentamycin/erythromycin. Double cross-out recombinants were screened via PCR for mutant genotype.

Genes expressed in trans, including those used to complement the mutants described above, were PCR amplified and ligated into BamHI-digested pFD340 (38) and conjugally transferred from E. coli to Bacteroidales.

### 5. Identification of Immunity Gene

The three genes unique to pBCPT that are not contained into pBUN24 (CS034_04056, CS034_04057, and CS034_04059) (FIG. 2g) were cloned individually into expression vector pFD340 using NEBuilder. The resulting plasmids were conjugally transferred into PvCL10 and the ability of the cloned product to protect PvCL10 from growth inhibition by activated BcpT was quantified in overlay assays compared to PvCL10 with vector alone.

6. pBCPT Mobilization Assays

To determine if pBCPT could be mobilized to other Bacteroidales species, the inventors started by making the donor strain (PvCL04) erythromycin sensitive and added tetQ to pBCPT. First, ermG was deleted from PvCL04 by amplifying regions upstream and downstream of ermG (CS034_04137) and cloning them into BamHI-digested pLGB30 (35) using NEBuilder. Cointegrates were selected on tetracycline/gentamycin and double cross-outs were selected by plating on BHIS with 10 mM rhamnose. Next, tetQ was amplified from with its own promoter from strain B. caccae CL03T12C61. The strong transcriptional termination region (ttr) downstream of BF638R_1994 was PCR amplified for placement downstream of tetQ to prevent transcriptional readthrough into other pBCPT genes. The tetQ-ttr was inserted between the two pBCPT divergently transcribed genes CS034_04061-62 in a manner so as not to affect the promoters of either gene (FIG. 7b). The DNA regions flanking each side of this insertion site were PCR amplified and the four PCR products (left flank, tetQ, ttr, right flank) were cloned directionally into BamHI-digested pLGB13. The correct assembly of these pieces was confirmed by whole plasmid sequencing and the plasmid was conjugally transferred into PvCL04 ΔermG and cointegrates were selected on erythromycin/gentamycin. Double cross outs were selected using BHIS plates containing aTC.

For recipient strains, the inventors used B. ovatus D2, which is ErmR and TetS, and constructed an erythromycin resistant strain of B. fragilis 638RΔT6SS (39), which lacks the ability to kill by the GA3 T6SS. ermG was added to the intergenic region between convergently transcribed genes BF638R_0867 and 0868. For this, ermG with its upstream promoter was amplified from cloning vector pNBU2-bla-ermG. Regions flanking the BF638R_0867 and 0868 insertion site were cloned on each side of ermG in vector pLGB36 that was PCR amplified to remove its ermG gene. The correct plasmid was confirmed by whole plasmid sequencing and conjugally transferred into 638RΔT6SS with cointegrates and double cross-outs selected as described above.

Donor strain BcvCL04ΔermGpBCPT-tetQ and recipient strains 638RΔT6SSermG and B. ovatus D2 were grown in basal medium until an OD₆₀₀ of 0.7. 100 μl of donor and 20 μl of recipient (5:1 donor recipient) were mixed and 10 μl was plated on a BHIS plate for 20 hours. The co-culture spot was cut from the plate and resuspended in 1 ml basal and 10-fold serial dilutions were plated on BHIS with erythromycin (number of recipients) or erythromycin+tetracycline (transconjugants). Transconjugants were passaged twice, and PCR was performed to confirm that the pBCPT transferred to the recipient strain. The multiplex PCR is based on the 16S rRNA gene and produces different sized products for different Bacteroidales species (40).

### 7. His-DpnA, His-DpnB, His-BSAP-3, His-BcpT and Site Mutants

BcpT-encoding gene (CS034_04058) was PCR amplified removing the N-terminal signal sequence and ligated into expression vector pET16b (Novagen) to produce an N-terminal His-tag. Point mutations in this His-BcpT construct were generated via Quickchange mutagenesis (Stratagene) and confirmed by DNA sequencing. BSAP-3-encoding gene (HMPREF1058_01765) was PCR amplified removing the N-terminal signal sequence and cloned into NdeI digested pET16b. Dpn genes lacking signal peptidase II sequences with a C-terminal polyhistidine (6×) tag were codon optimized for expression in E. coli and cloned into pET21b+ via the NdeI and XhoI sites (Genscript).

Purification of polyhistidine-tagged proteins was performed as previously described (41). Briefly, an overnight culture of E. coli BL21 (DE3) cells containing the plasmid encoding the protein of interest was used to inoculate 1.7 L culture of Terrific Broth (TB, EMD Millipore) supplemented with the appropriate antibiotics. The culture was induced via 0.5 mM isopropyl β-D-1-thiogalactopyranoside (IPTG, Gold Biosciences) at an OD₆₀₀˜1.0 and incubated at 37° C. for 5 hours with the exception of Dnp2, which required expression at 18° C., possibly due to it being proteolytically active on E. coli cells upon expression. The cells were spun down and resuspended in buffer A (10 mM MES pH 6.5, 150 mM NaCl) and the cells lysed by 3 passages through and Emulsiflex C3 cell disruptor (Avestin) at 15,000 psi. Cell debris was removed via centrifugation (14,000×g for 20 min) and the clarified lysate was recirculated in a column containing 10 ml of metal-chelating sepharose (GE) charged with cobalt. The polyhistidine-tagged proteins were eluted from the column using a 0-50% buffer B gradient (10 mM MES pH 6.5, 1 M imidazole, 90 mL total gradient volume). The fractions containing the purified protein were pooled, concentrated, exchanged into Tris buffered saline (TBS), pH 8.0 with 10% glycerol, and flash frozen in liquid nitrogen prior to storage at −80° C.

### 8. Enzymatic Activation of BcpT and Proteases

Recombinant, purified BcpT was activated using Factor Xa (Millipore) following the supplier instructions. Briefly, a 1:200 Factor Xa to BcpT (w:w) ratio was added into TBS pH 8.0, 5 mM Ca²⁺⁺ and incubated for 72 hours at RT to achieve complete cleavage of BcpT, as measured by the absence of uncleaved BcpT detected via Western blot. Factor Xa was then removed from sample via supplied affinity beads. All FXa activated BcpT used as active toxin in this study was cleaved to completion using this method.

DpnA eluted as a proenzyme and presumably requires cleavage of a putative activation loop, similar to that found in fragipain (20) for activity. Trypsin was added to DpnA at a 1:50 (w:w) ratio and incubated at 37° C. for 15 minutes. Trypsin was inactivated by bringing the sample to 1 mM phenylmethylsulphonyl fluoride (PMSF). Complete cleavage and proteolytic activation of BcpT was achieved by adding activated DpnA to BcpT at a 1:25 (w:w) ratio and incubating at room temperature for 2 hours. DpnB eluted in an active form and did not require treatment with trypsin for activation. Proteolytic activation of BcpT by DpnB was achieved by adding activated DpnB to BcpT at a 1:25 ratio (w:w) and incubated at room temperature for 2 hours.

His-BSAP-3 was activated using a 5:1 BSAP-3:DpnB ratio and incubating at 37° C. for 3 hours in TBS, pH 8.0.

9. BcpT Site Mutants for Expression in PdCL02 ΔbcpT

To study the effect of mutation of R65 and R199 when expressed from P. dorei, the R65N, R199N, and double R65N-R199N site mutants of bcpT were constructed using the His-tagged constructs as template (see above) removing the His-tag and adding the native ribosome binding site and signal sequence and expressed using pFD340. The native bcpT cloned into pFD340 was used as template for the rbs and the signal sequence, and the His-tagged clones were used as template for the native and three site mutants of BcpT. These pieces were cloned directionally into BamHI-digested pFD340 using NEBuilder. All plasmids were verified by whole plasmid sequencing and conjugally transferred to PdCL02 ΔbcpT.

### 10. Antiserum Generation and Western Blot Analysis

Antiserum to BcpT was generated in rabbits at Lampire Biological Laboratories using the Expressline protocol with purified His-tagged BcpT as the immunogen. Use of rabbits for antiserum generation was approved by the Institutional Animal Care and Use Committee (IACUC), Brigham & Women's Hospital and complies with all relevant ethical regulations for animal testing and research. Antibodies specific to His-BcpT were purified by conjugating 10 mg of purified recombinant His-BcpT to 1 ml of Affi-Gel 15 beads (Bio-Rad) following the manufacturer's instructions. Rabbit anti-BcpT was recirculated through this affinity column for 30 minutes at room temperature. The beads were washed with 10 column volumes of 10 mM Tris, pH 8.0, 1 M NaCl and eluted using 50 mM glycine, pH 2.5. One ml fractions were collected into tubes containing 200 μL of 1M Tris, pH 8.0 to neutralize the fractions. The antibody was dialyzed into HBS (50 mM HEPES, 100 mM NaCl, pH 7.4) and stored at 4° C. The antiserum to the O-antigen of PvCL10 was generated previously (3).

Proteins from SDS-PAGE gels were electrophoretically transferred to nitrocellulose paper and then blocked with blocking buffer (3% skim milk in blot buffer (100 mM Tris-HCl, pH8.0, 150 mM NaCl) for 1 hour. Affinity purified α-BcpT was then added at a 1:1000 dilution and incubated overnight at 4° C. with rocking. The membrane was then washed 3 times for 10 min with 30 ml of blot buffer+0.1% Tween 20. Secondary antibody (α-rabbit IgG-HRP conjugate) was then added at a 1:500 dilution to 20 ml blot buffer+0.1% Tween 20 and incubated for I hour and then washed as above. Bands were identified by chemiluminescence using ECL Western blotting reagents (Millipore) and imaged on Blublot HS film Life Science Products).

### 11. N-Terminal Sequencing of BcpT Fragments

His-tagged BcpT was treated with Factor Xa and the fragments were separated on 12% MES gel under non-reducing conditions. After transfer of the contents of the gel to a PVDF membrane, the membrane was washed in distilled water, strained with 0.02% Coomassie Brilliant blue in 40% methanol, 5% acetic acid for 20-30 seconds, destained in 40% methanol, 5% acetic acid for 1 min, and finally rinsed in distilled water. Fragments of approximately 35 kDa, 25 kDa and 15 kDa were cut from the membrane and sent for N-terminal sequencing at Tufts Core Facility. Five N-terminal residues were identified for each fragment.

### 12. Fluorescent Labeling of BcpT

Cysteine modification of BcpT was performed as previously described (42). Briefly, BcpT was incubated with a 20-fold molar excess of Alexa Fluor 488 (Invitrogen) overnight at 4° C. Free dye was removed from the labeled sample using a G-50 (GE), and sample concentration was assessed via colorimetric assay. Absorbance of fluorophore at 495 nm was obtained and labeling efficiency was calculated using the molar extinction coefficient (73,000 cm⁻¹M⁻¹).

### 13. Membrane Purification

Cells for membrane purifications from various Bacteroidales strains were grown to OD₆₀₀=0.8 and centrifuged at 14,000×g for 30 minutes. Cells were resuspended in HBS (10 mM HEPES, pH 7.4, 150 mM NaCl) and passed through a cell disruptor (Avestin) 3 times at 15,000 psi. Cell debris was removed via centrifugation at 7,000×g for 10 minutes, and the supernatant was further centrifuged at 530,000×g. Membranes were resuspended in HBS using a Dounce homogenizer.

### 14. Purified LPS and Membrane Receptor Blots

LPS was purified by the hot phenol method previously described (43). Purified LPS from E. coli 0111:B4 and Salmonella typhimurium were purchased from List Labs. Purified membranes or LPS samples were separated on a 4-20% SDS-PAGE gradient gel prior to transfer to nitrocellulose membranes. Proteins and lipids were electrophoretically transferred to nitrocellulose, which were incubated with western blot blocking buffer prior to probing them with purified activated or unactivated BcpT (30 g total at 1.5 g/ml in 3% skim milk in blot buffer) with or without Alexa Fluor 488 label and incubated overnight at 4° C. Blots incubated with fluorescently labeled protein were washed 3 times for 10 minutes in blot buffer containing 0.1% Tween 20 and imaged immediately using a Chemidoc 10 imaging system (BioRad). Blots treated with unlabeled protein were washed as above and then incubated with affinity-purified α-BcpT for 2 hours at room temperature in blocking buffer. The blot was washed as above and then incubated with HRP conjugated secondary antibody (Invitrogen) in blot wash buffer for 1 hour at room temperature. After washing the blot as described above the blots were visualized with ECL Western blotting reagents (Millipore) per the supplier's instructions and imaged on film as described above for Western blots.

### 15. Genome Sequencing

The genomes of PvCL04 and PvCL05 were sequenced by the Biopolymers Facility, Harvard Medical School, Boston, MA, using the Illumina MiSeq platform, generating paired-end reads of 150 bp. Adapter sequences were removed, and the reads were quality trimmed using BBDuk (a component of the BBTools program suite distributed by the Department of Energy's Joint Genome Institute; found on the world wide web at jgi.doe.gov/data-and-tools/bbtools/). Reads were screened for vector contamination using NCBI's UniVecCore collection (build 10.0, with entries originating in GenBank removed) and reads returning a significant hit were discarded. De novo assembly was performed using Velvet Optimizer and Velvet. Gene calling was performed using Prodigal 2.6.3 and annotation was performed using a customized version of Prokka 1.14.6.

Long-read sequencing of PvCL10 was performed by SNPsaurus (Institute of Molecular Biology, 1318 Franklin Blvd, Room 273 Onyx Bridge, Eugene, OR) using the PacBio Seqel II HiFi platform, and short-read sequencing was performed by the Biopolymers Facility, Harvard Medical School, Boston, MA, using the Illumina MiSeq platform. Assembly of the genome was conducted using the Flye assembler (version 2.9 (44)) The genome was further polished by mapping the Illumina reads and the PacBio HiFi (css) reads to the assembled genome using Snippy (v4.60, Torsten Seemann, University of Melbourne, Australia, found on the world wide web at github.com/tseemann/snippy) in successive rounds and correcting the variations found.

### 16. Analysis of Genomic and Metagenomic Datasets

The locally curated set of 1434 Bacteroidales genomes (described in (45)) was modified by removing entries not identified to the species level to create subset database comprising 1148 genomes. This database was utilized for detection (using Blastn 2.10.0+) of homologs to various segments of the 9,177 bp pBCPT plasmid: a backbone query comprising 5,058 bp (join(1 . . . 2280, 6340 . . . 9117)), and the 1,500 bp bcpT toxin gene sequence alone (CS034_04058, 4672 . . . 6171). Blastn returns for all queries were retained if the percent ID was ≥75% over on an alignment≥500 bp (for the three gene query) or ≥1000 bp (for the backbone query).

Analyses of metagenomic datasets for the presence and geographical distribution of the bcpT gene utilized the inventors' collection of 15 publicly available human gut metagenomic datasets comprising samples collected from 1,767 individuals. This metagenomic collection and the methodology used were previously described. (45).

### 17. RNASeq Analysis

For RNASeq analysis, triplicate cultures of PvCL10 were grown to an OD₆₀₀ of 0.43-0.47 and BcpT was added at 7.5 μg/ml. Bacteria were harvested after 3 hours of BcpT treatment when their OD₆₀₀ was 0.080-0.082, compared to the untreated control which was OD₆₀₀ 1.35. Triplicate cultures of untreated bacteria were grown and harvested at OD₆₀₀ 0.80-0.84 as the untreated control samples. RNASeq of these six samples was performed by Novogene (South Plainfield, NJ) as 150 bp paired-end reads. For RNASeq analysis of PvCL10 tn79 and the WT PvCL10, bacteria were grown to an OD₆₀₀ of 0.6 and harvested. RNA sequencing was performed by the Molecular Biology Core Facilities, Dana Farber Cancer Institute as 75-bp paired-end reads, with samples and controls provided as biological duplicates.

Reads from all samples were adapter- and quality-trimmed using utilities included in the BBMap package of bioinformatics tools (v. 38.90) and mapped to PvCL10 using the Bowtie 2 short read aligner (v. 2.4.2) (46). SAMtools (v. 1.11) (47) was used to convert the Bowtie 2 output to sorted and indexed BAM files, and these were compared to General Feature Format (GFF) files of the intervals of protein-coding domains from the appropriate genome using BEDtools (v. 2.30.0) (48). Domains annotated as pseudogenes or as partial, truncated, or frameshifted genes were excluded. The read mapping results were evaluated for differential gene expression using both DESeq2 (v. 1.30.0) (49) and edgeR (v. 3.32.1) (50). The inventors considered a gene differentially expressed if the absolute value of its fold change (FC) in expression level under experimental conditions differed from the control conditions by ≥2 and if the adjusted p-value (padj for DESeq2 and FDR for edgeR) was ≤0.05, as calculated by both statistical packages. In cases where DESeq2 returned NA due to read count outlier detection, edgeR calculations were relied on exclusively for determination of differential expression.

Cross comparison of the PvCL10 RNASeq data to the published Pv8482 RNASeq data of bacteroidetocin A treated cells (8) proceeded by first finding the reciprocal best hits (RBH) between the proteomes of the two P. vulgatus isolates. RBH analysis utilized blastp (v. 2.11.0) using settings as suggested by⁵¹ (e.g. -evalue 10⁻⁶, -seg yes, -soft_masking true, and -comp_based_stats 0).

### 18. Data Availability

P. vulgatus CL04T12C01 and P. vulgatus CL05T12C02 genome sequences were deposited in GenBank under BioProject accession number PRJNA415639. The P. vulgatus CL10T00C06 genome was deposited in GenBank under BioProject accession number PRJNA830856. RNASeq data for both the BcpT exposure experiments and the transposon mutant experiments were deposited as BAM files in the SRA, also linked to BioProject PRJNA830856. DGE values with statistics from the RNASeq analyses for all PvCL10 genes from the untreated versus BcpT treated samples are provided in Supplementary Data 1.

### D. Tables

SUPPLEMENTARY TABLE 1
Comparison of proteins encoded by pBUN24 and pBCPT

| | | | | | | |
|---|---|---|---|---|---|---|
| Gene name | Product | Motifs | Start | Stop | bp | AA |

pBUN24

| | | | | | | |
|---|---|---|---|---|---|---|
| repA | repA | PF01051 | 476 | 1453 | 978 | 325 |
| CDS_9 (orf2) | Hypothetical protein | | 1467 | 1649 | 183 | 60 |
| CDS_8 (orf1) | Hypothetical protein | | 1763 | 2284 | 522 | 173 |
| CDS_7 (orf7)** | Hypothetical protein | | 2314 | 2855 | 542 | 180 |
| CDS_6 (orf6)** | HTH transcriptional | | 2878 | 3975 | 1098 | 365 |
| | regulator | | | | | |
| CDS_5 (orf5)** | Putative lipoprotein | PF08450 | 4195 | 5850 | 1656 | 551 |
| mobB | MobB | | 6405 | 7283 | 879 | 292 |
| mobA | MobA | | 7280 | 7885 | 606 | 201 |
| CDS_4 (orf4) | Hypothetical protein | | 7917 | 8183 | 267 | 88 |
| CDS_3 (tad) | TA system toxin | PF05973, | 8277 | 8624 | 348 | 115 |
| | | TIGR030070 | | | | |
| CDS_2 (ata) | TA system anti-toxin | PF15731 | 8630 | 8944 | 315 | 104 |
| CDS_1 (orf3) | Hypothetical protein | | 8938 | 263 | 270 | 89 |

pBCPT (PvCL04 gene designations)

| | | | | | | |
|---|---|---|---|---|---|---|
| CS034_04053 | repA | PF01051 | 475 | 1452 | 978 | 325 |
| (92.62% similarity | | | | | | |
| to repA*) | | | | | | |
| CS034_04054 | Hypothetical protein | | 1466 | 1657 | 192 | 63 |
| (96.36% similarity | | | | | | |
| to CDS_9*) | | | | | | |
| CS034_04055 | Hypothetical protein | | 1759 | 2280 | 522 | 173 |
| (94.80% similarity | | | | | | |
| to CDS_8*) | | | | | | |
| CS034_04056** | LuxR-type DNA-binding | PF00196 | 2301 | 3938 | 1638 | 545 |
| | HTH domain protein | | | | | |
| CS034_04057** | Predicted OMP | | 4086 | 4658 | 573 | 190 |
| CS034_04058** | BcpT (experimental | | 4672 | 6171 | 1500 | 499 |
| | evidence) | | | | | |
| CS034_04059** | BcpI (experimental | | 6228 | 6401 | 174 | 57 |
| | evidence) | | | | | |
| CS034_04060 | MobB | DUF5712, | 6579 | 7457 | 879 | 292 |
| (98.29% similarity | | PF18976 | | | | |
| to mobB*) | | | | | | |
| CS034_04061 | MobA | PF01726 | 7454 | 8059 | 606 | 201 |
| (99.50% similarity | | | | | | |
| to mobA*) | | | | | | |
| CS034_04062 | HipB-like toxin | PF05973, | 8450 | 8797 | 348 | 115 |
| (98.26% similarity | | TIGR03070 | | | | |
| to CDS_3*) | | | | | | |
| CS034_04063 | MqsA-like anti-toxin | PF15731 | 8803 | 9117 | 315 | 104 |
| (100.00% similarity | | | | | | |
| to CDS_2*) | | | | | | |

CDS names are from NCBI GenBank EU818711 file, pBUN24 names are from FIG. 1 in Shkoporov A N, et al. 2013. Analysis of a novel 8.9 kb cryptic plasmid from Bacteroides uniformis, its long-term stability and spread within human microbiota. Plasmid 69(2): 146-59.
*percent similarity to indicated gene in the pBUN24 plasmid.
**genes unique between each of the two plasmids.

SUPPLEMENTARY TABLE 2
Primers used in this Example

| | | | SEQ |
|---|---|---|---|
| | | | ID |
| | | | NO |
| Construction of | left flank | caagggatccataagaaaaaggcacatcgagaac | 17 |
| PvCL04 and PdCL02 | forward | | |
| ΔbcpT | left flank | gtcgacgcgtttgttacatgaacacaaaatagctg | 18 |
| | reverse | | |
| | right flank | atgtacgcgttttgttggtaagccagaaattgta | 19 |
| | forward | | |
| | right flank | cgaaggatccaagatcttctcctatatcgcaagc | 20 |
| | reverse | | |
| Clone bcpT into | forward | accaggatccaaacacggtataacaaaagaacca | 21 |
| pFD340 | reverse | aggaggatccactccttccctattttgtttagga | 22 |
| Screening for bcpT in | forward | ggtatatactatggagttgc | 23 |
| unsequenced strains | reverse | actccttccctattttgtttagga | 24 |
| Outward primers for | left junction | gtggatatttgttttccaacatga | 25 |
| circular plasmid | primer | | |
| confirmation | right junction | aagataaagcgtatggaagcaaag | 26 |
| | primer | | |
| Construction of His- | forward | acatcatatgaacaatgaacttcctattgaaaatgtaca | 27 |
| BcpT for expression in | reverse | aggaggatccactccttccctattttgtttagga | 28 |
| E. coli | | | |
| His-BcpT (R65N) | forward | gataatcctattttaacaaacgcaagttcatcttttg | 29 |
| | reverse | caaaagatgaacttgcgtttgttaaaataggattatc | 30 |
| His-BcpT (R199N) | forward | gggactgaaactctcacaaactccgcaagcgatggaggt | 31 |
| | reverse | acctccatcgcttgcggagtttgtgagagtttcagtccc | 32 |
| Amplify native and | forward | gtgttcatgtaacaatgaacttcctattgaaaat | 33 |
| site mutants of bcpT | reverse | attcgagctcggtacccgggactccttccctattttgtttag | 34 |
| from His-constructs | | | |
| Amplify rbs and signal | forward | aatcagaattgactctagagcacggtataacaaaagaacc | 35 |
| sequence of bcpT for | reverse | gttcattgttacatgaacacaaaatagctg | 36 |
| construction of bcpT | | | |
| site mutants in | | | |
| pFD340 | | | |
| Construction of | left flank | taagattagcattatgagtggcatctaagatagctgac | 37 |
| PdCL02 Δdpn1 | forward | | |
| (HMPREF1064_01439) | left flank | ctttataccagccacatactaataacagaaaac | 38 |
| BamHI digested | reverse | | |
| pLGB13 | right flank | agtatgtggctggtataaagatactaatacacattagag | 39 |
| | forward | | |
| | right flank | cgaattcctgcagcccgggggctaactcagatgctcgc | 40 |
| | reverse | | |
| Construction of | left flank | taagattagcattatgagtgggtaggctcgttgaacaag | 41 |
| PdCL02 Δdpn2 | forward | | |
| (HMPREF1064_00451) | left flank | cagcccatcccataagaaaaggatcactcttcc | 42 |
| BamHI digested | reverse | | |
| pLGB13 | right flank | ttttcttatgggatgggctgaaacggaatc | 43 |
| | forward | | |
| | right flank | cgaattcctgcagcccggggaagcaatgatggctctttctatc | 44 |
| | reverse | | |
| Complement PdCL02 | forward | aatcagaattgactctagagtcaagaaaccggaagagtg | 45 |
| Δdpn2 (in pFD340) | reverse | attcgagctcggtacccgggctggctgcattctccttc | 46 |
| Deletion of PvCL10 | left flank | cggtgtaagattagcattatgagtgtccgcttcatggagactc | 47 |
| M0N98_03760-61 | forward | | |
| (sigma | left flank | agcaatgacacggtgagcttgcacgtacaaac | 48 |
| factor/antisigma | reverse | | |
| factor) | right flank | acgtgcaagctcaccgtgtcattgctccttc | 49 |
| | forward | | |
| | right flank | gatatcgaattcctgcagcccgggggtttcgcggtcactcatc | 50 |
| | reverse | | |
| Clone | forward | ttctggatcccggatgcagtatttttgcagtctt | 51 |
| M0N98_03761into | reverse | ctacggatccggttccgaatagccctccatc | 52 |
| pFD340 | | | |
| Clone | forward | gatgggatcctacagagccaaaaccttattgatg | 53 |
| M0N98_03760 into | reverse | tcatggatccattttatataagaacagtgaaaaagttcat | 54 |
| pFD340 | | | |
| Clone | forward | ttctggatcccggatgcagtatttttgcagtctt | 55 |
| M0N98_03760-61 | reverse | tcatggatccattttatataagaacagtgaaaaagttcat | 56 |
| into pFD340 | | | |
| Construction of | left flank | agtggatcccccgggctgcaccaaaatagggcacgttatatc | 57 |
| Pv8482 O-ag deletion | forward | | |
| mutant Bvu_1068-69 | left flank | tctttatgattacctccagtaccggatattc | 58 |
| cloned into PstI site | reverse | | |
| pLGB13 | right flank | actggaggtaatcataaagaaatggatggaagtgatc | 59 |
| | forward | | |
| | right flank | cttgatatcgaattcctgcagtggatgatccggagctg | 60 |
| | reverse | | |
| Construction of His- | forward | taaacatatgtgtacaaatacttatgacgtttgtgaaat | 61 |
| BSAP-3 for | reverse | ccaccatatgttatggataaacatatcctaaaataccacc | 62 |
| expression in E. coli | | | |
| Construction of | left flank | taagattagcattatgagtgcgtaggcaaaaacggcatag | 63 |
| PvCL04 ermG | forward | | |
| deletion mutant into | left flank | ctttgaactacattagtaacttcttacaggtgaatac | 64 |
| BamHI site pLGB30 | reverse | | |
| | right flank | gttactaatgtagttcaaagtcgggtgg | 65 |
| | forward | | |
| | right flank | cgaattcctgcagcccggggcgacacggttatttgggatag | 66 |
| | reverse | | |
| Construction of | forward | ttcgtcgattcttgccagttgaacctac | 67 |
| pBCPT-tetQ PCR | reverse | aattctgttcccgtattgccttatagaaatttc | 68 |
| amplify | | | |
| BcCL03T12C61 tetQ | | | |
| PCR amplify | forward | ggcaatacgggaacagaattggccgaag | 69 |
| BF638R_1994 | reverse | actgccgagcccaccaattccatattcaag | 70 |
| transcriptional stop | | | |
| PCR amplify pBCPT | left flank | taagattagcattatgagtgggtcgaacttataacgggc | 71 |
| flanks for tetQ | forward | | |
| integration | left flank | aactggcaagaatcgacgaacaccctcc | 72 |
| | reverse | | |
| | right flank | gaattggtgggctcggcagtcgaaagaac | 73 |
| | forward | | |
| | right flank | cgaattcctgcagcccgggggtgacctcccttgcgctc | 74 |
| | reverse | | |
| Insertion of ermG in | forward | tcttctaaccgcttgacggaaatcaacaaattttag | 75 |
| B. fragilis | reverse | ttgcactacgcagtttgatttctcaggactttac | 76 |
| 638RΔT6SS PCR | | | |
| amplify ermG from | | | |
| pNBU2-erm | | | |
| PCR amplify flanking | left flank | gctcaacaattgcttgacgggtggagctgatgcgtttc | 77 |
| regions to insert ermG | forward | | |
| | left flank | tccgtcaagcggttagaagaattgtggaagtc | 78 |
| | reverse | | |
| | right flank | aatcaaactgcgtagtgcaaatatagcg | 79 |
| | forward | | |
| | right flank | tgcctgttctatttccgaaccgatgtagccattatcgatg | 80 |
| | reverse | | |
| amplify pLGB36 | forward | gttcggaaatagaacagg | 81 |
| removing plasmid | reverse | ccgtcaagcaattgttgag | 82 |
| ermG | | | |
| Detect pBCPT in | forward | aatcagaattgactctagagcgctccttcccttcacttattg | 83 |
| transconjugants | reverse | attcgagctcggtacccgggcattggtgatgatgctatttaagatag | 84 |
| orward multiplex | MPI forward | gtacacaccgcccgt | 85 |
| primer (for all three | | | |
| species) | | | |
| multiplex P. vulgatus | | ctttctctcttccgtatcattac | 86 |
| reverse | | | |
| Multiplex B. fragilis | MPI-Bf | gctaatcccccaatcatac | 87 |
| reverse | reverse | | |
| multiplex B. ovatus | MPI-Bo | atcaatattgcgtactcgaac | 88 |
| reverse | reverse | | |
| Clone PvCL04 | forward | aatcagaattgactctagagctgagttgtaatcaaacctattg | 89 |
| CS034_04056 into | reverse | attcgagctcggtacccggggatagttaaggtgcatcac | 90 |
| pFD340 | | | |
| Clone PvCL04 | forward | aatcagaattgactctagaggtctaactttgcaacgttc | 91 |
| CS034_04057 into | reverse | attcgagctcggtacccgggttaaaatgaatatccaaaactaatataag | 92 |
| pFD340 | | g | |
| Clone PvCL04 | forward | aatcagaattgactctagagcgctccttcccttcacttattg | 93 |
| CS034_04059 into | reverse | attcgagctcggtacccgggcattggtgatgatgctatttaagatag | 94 |
| pFD340 | | | |

SUPPLEMENTARY TABLE 3

| | | Toxin phenotype of |
|---|---|---|
| | | wild type Pv and Pd |
| Strains used in this study | Source | strains |
| Phocaeicola dorei CL02T00C15 | Comstock lab | BcpT |
| | (Zitomersky, 2011) | |
| Phocaeicola dorei CL02T00C15 | this study | |
| ΔbcpT (ΔHMPREF1063_05166) | | |
| Phocaeicola dorei CL02T00C15 | this study | |
| ΔbcpT pbcpT | | |
| Phocaeicola dorei CL02T00C15 | this study | |
| ΔbcpT pbcpT R6SN | | |
| Phocaeicola dorei CL02T00C15 | this study | |
| ΔbcpT pbcpT R199N | | |
| Phocaeicola dorei CL02T00C15 | this study | |
| ΔbcpT pbcpT R6SN R199N | | |
| Phocaeicola vulgatus CL04T12C01 | Comstock lab | BcpT |
| | (Zitomersky, 2011) | |
| Phocaeicola vulgatus CL04T12C01 | this study | |
| tn10 | | |
| Phocaeicola vulgatus CL04T12C01 | this study | |
| tn12 | | |
| Phocaeicola vulgatus CL04T12C01 | this study | |
| ΔbcpT (ΔCS034_04058) | | |
| Phocaeicola vulgatus CL04T12C01 | this study | |
| ΔbcpT pbcpT | | |
| Phocaeicola vulgatus CL04T12C01 | this study | |
| ΔermG pBCPT-tetQ-ttr | | |
| Phocaeicola dorei CL03T12C01 | Comstock lab | BSAP-3 |
| | (Zitomersky, 2011) | |
| Phocaeicola dorei 9_1_42 FAA | BEI | BSAP-3 |
| Phocaeicola dorei 5_1_36/D4 | BEI | |
| Phocaeicola dorei CL01T00C33 | Comstock lab | |
| | (Zitomersky, 2011) | |
| Phocaeicola dorei CL08T03C21 | Comstock lab | |
| | (Zitomersky, 2011) | |
| Phocaeicola dorei CL12T00C02 | Comstock lab | |
| | (Zitomersky, 2011) | |
| Phocaeicola dorei CL15T00C36 | Comstock lab | |
| | (Zitomersky, 2011) | |
| Phocaeicola vulgatus ATCC 8482 | ATCC | |
| Phocaeicola vulgatus ATCC 8482 | this study | |
| ΔBvu 1068-69 (Q-ag mutant) | | |
| Phocaeicola vulgatus CL05T12C02 | Comstock lab | BcpT |
| | (Zitomersky, 2011) | |
| Phocaeicola vulgatus CL06T12C01 | Comstock lab | |
| | (Zitomersky, 2011) | |
| Phocaeicola vulgatus CL07T06C01 | Comstock lab | |
| | (Zitomersky, 2011) | |
| Phocaeicola vulgatus CL09T03C04 | Comstock lab | |
| | (Zitomersky, 2011) | |
| Phocaeicola vulgatus CL10T00C06 | Comstock lab | BSAP-3 |
| | (Zitomersky, 2011) | |
| Phocaeicola vulgatus CL10T00C06 | Comstock lab (Laclare | |
| ΔCK234_00400-401(O-ag mutant) | McEneanv. 2018) | |
| Phocaeicola vulgatus CL10T00C06 | this study | |
| ΔCK234_00923-924 (Δsig/antisig) | | |
| Phocaeicola vulgatus CL10T00C06 | this study | |
| ΔCK234_00923-924 p00923 | | |
| Phocaeicola vulgatus CL10T00C06 | this study | |
| ΔCK234 00923-924 p00924 | | |
| Phocaeicola vulgatus CL10T00C06 | this study | |
| pBCPT gene (CS034_04056) | | |
| Phocaeicola vulgatus CL10T00C06 | this study | |
| pBCPT gene (CS034_04057) | | |
| Phocaeicola vulgatus CL10T00C06 | this study | |
| pBCPT gene (CS034_04056) | | |
| Phocaeicola vulgatus CL11T12C09 | Comstock lab | |
| | (Zitomersky, 2011) | |
| Phocaeicola vulgatus CL13T00C01 | Comstock lab | |
| | (Zitomersky, 2011) | |
| Phocaeicola vulgatus CL14T03C19 | Comstock lab | |
| | (Zitomersky, 2011) | |
| Bacteroides thetaiotamicron VPI 5482 | lab stock | |
| Bacteroides thetaiotamicron VPI 5482 | Comstock lab (Ladare | |
| pFD340 | McEneany, 2018) | |
| Bacteroides thetaiotamicron VPI 5482 | this study | |
| pbcpT | | |
| Bacteroides ovatus D2 | BEI | |
| Bacteroides ovatus D2 pBCPT-tetO-ttr | this study | |
| Phocaeicola dorei CL02T12C06 | Comstock lab | BcpT |
| | (Zitomerskv. 2011) | |
| Phocaeicola dorei CL02T12C06 | this study | |
| Δdpn1 | | |
| Phocaeicola dorei CL02T12C06 | this study | |
| Δdpn2 | | |
| Phocaeicola dorei CL02T12C06 | this study | |
| Δdpn1 Δdpn2 | | |
| Bacteroides fragilis 638R. | lab stock | |
| Bacteroides fragilis 638RΔT6SSermG | this study | |
| Bacteroides fragilis 638RΔT6SSermG | this study | |
| pBCPT-tetQ-ttr | | |
| Bacteroides ovatus ATCC 8483 | ATCC | |
| Bacteroides uniformis ATCC 8492 | ATCC | |
| Bacteroides finegoldii CL09T03C10 | Comstock lab | |
| | (Zitomersky, 2011) | |
| Parabacteroiees merdae CL03T12C32 | Comstock lab | |
| | (Zitomersky, 2011) | |
| Parabacteroiees johnsonii | Comstock lab | |
| CL03T12C29 | (Zitomersky, 2011) | |
| Parabacteroiees goldsteinii | Comstock lab | Bacteroidetocin A, |
| CL03T12C30 | (Zitomersky, 2011) | BcpT |
| E. coli DH5α | lab collection | |
| E. coli BL21/DE3 | lab collection | |
| E. coli S17 λA.llir | E. Martens | |
| E. coli HB101 | lab collection | |
| E. coli HS | lab collection | |
| E. coli BL21/DE3 pHis-bcpT | this study | |
| E. coli BL21/DE3 pHis-bcpT R65N | this study | |
| E. coli BL21/DE3 pHis-bcpT R199N | this study | |
| E. coli BL21/DE3 pHis-bcpT R65N | this study | |
| R199N | | |
| E. coli BL21/DE3 pHis-dpn1 | this study | |
| E. coli BL21/DE3 pHis-dpn2 | this study | |
| Salmonella enterica ATCC 14028 | ATCC | |

Zitomersky N L, Coyne M J, Comstock L E. Longitudinal analysis of the prevalence, maintenance, and IgA response to species of the order Bacteroidales in the human gut. Infect Immun. 2011 May; 79(5):2012-20. doi: 10.1128/IAI.01348-10. Epub 2011 Mar. 14. PMID: 21402766; PMCID: PMC3088145.
McEneany V L, Coyne M J, Chatzidaki-Livanis M, Comstock L E. Acquisition of MACPF domain-encoding genes is the main contributor to LPS glycan diversity in gut Bacteroides species. ISME J. 2018 December; 12(12):2919-2928. doi: 10.1038/s41396-018-0244-4. Epub 2018 July 31. PMID: 30065309; PMCID: PMC6246601.
Roelofs K G, Coyne M J, Gentyala R R, Chatzidaki-Livanis M, Comstock L E. Bacteroidales Secreted Antimicrobial Proteins Target Surface Molecules Necessary for Gut Colonization and Mediate Competition In Vivo. mBio. 2016 Aug. 23; 7(4):e01055-16. doi:10.1128/mBio.01055-16. PMID: 27555309; PMCID: PMC4999547.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
1 Chatzidaki-Livanis, M., Coyne, M. J. & Comstock, L. E. An antimicrobial protein of the gut symbiont Bacteroides fragilis with a MACPF domain of host immune proteins. Mol Microbiol 94, 1361-1374, doi:10.1111/mmi.12839 (2014).
2 Roelofs, K. G., Coyne, M. J., Gentyala, R. R., Chatzidaki-Livanis, M. & Comstock, L. E. Bacteroidales Secreted Antimicrobial Proteins Target Surface Molecules Necessary for Gut Colonization and Mediate Competition In Vivo. mBio 7, doi:10.1128/mBio.01055-16 (2016).
3 Laclare McEneany, V., Coyne, M. J., Chatzidaki-Livanis, M. & Comstock, L. E. Acquisition of MACPF domain-encoding genes is the main contributor to LPS glycan diversity in gut Bacteroides species. ISME J 12, 2919-2928, doi:10.1038/s41396-018-0244-4 (2018).
4 Shumaker, A. M., Laclare McEneany, V., Coyne, M. J., Silver, P. A. & Comstock, L. E. Identification of a Fifth Antibacterial Toxin Produced by a Single Bacteroides fragilis Strain. J Bacteriol 201, doi:10.1128/JB.00577-18 (2019).
5 Coyne, M. J. et al. A family of anti-Bacteroidales peptide toxins wide-spread in the human gut microbiota. Nature communications 10, 3460, doi:10.1038/s41467-019-11494-1 (2019).
6 Bao, Y. et al. A Common Pathway for Activation of Host-Targeting and Bacteria-Targeting Toxins in Human Intestinal Bacteria. mBio 12, e0065621, doi:10.1128/mBio.00656-21 (2021).
7 Chatzidaki-Livanis, M. et al. Gut Symbiont Bacteroides fragilis Secretes a Eukaryotic-Like Ubiquitin Protein That Mediates Intraspecies Antagonism. mBio 8, doi:10.1128/mBio.01902-17 (2017).
8 Matano, L. M., Coyne, M. J., Garcia-Bayona, L. & Comstock, L. E. Bacteroidetocins target the essential outer membrane protein BamA of Bacteroidales symbionts and pathogens. (submitted).
9 Garcia-Lopez, M. et al. Analysis of 1,000 Type-Strain Genomes Improves Taxonomic Classification of Bacteroidetes. Front. Microbiol. 10, 2083, doi:10.3389/fmicb.2019.02083 (2019).
10 Rigottier-Gois, L., Rochet, V., Garrec, N., Suau, A. & Dore, J. Enumeration of Bacteroides species in human faeces by fluorescent in situ hybridisation combined with flow cytometry using 16S rRNA probes. System App. Microbiol. 26, 110-118, doi:10.1078/072320203322337399 (2003).
11 Yang, J. et al. Species-Level Analysis of Human Gut Microbiota With Metataxonomics. Front. Microbiol. 11, 2029, doi:10.3389/fmicb.2020.02029 (2020).
12 Pedersen, R. M., Marmolin, E. S. & Justesen, U. S. Species differentiation of Bacteroides dorei from Bacteroides vulgatus and Bacteroides ovatus from Bacteroides xylanisolvens—back to basics. Anaerobe 24, 1-3, doi:10.1016/j.anaerobe.2013.08.004 (2013).
13 Avelar, K. E. et al. Production of bacteriocin by Bacteroides fragilis and partial characterization. Lett Appl Microbiol 29, 264-268 (1999).
14 Shkoporov, A. N. et al. Analysis of a novel 8.9 kb cryptic plasmid from Bacteroides uniformis, its long-term stability and spread within human microbiota. Plasmid 69, 146-159, doi:10.1016/j.plasmid.2012.11.002 (2013).
15 Fletcher, C. M., Coyne, M. J., Villa, O. F., Chatzidaki-Livanis, M. & Comstock, L. E. A general O-glycosylation system important to the physiology of a major human intestinal symbiont. Cell 137, 321-331 (2009).
16 Choi, V. M. et al. Activation of Bacteroides fragilis toxin by a novel bacterial protease contributes to anaerobic sepsis in mice. Nat Med 22, 563-567, doi: 10.1038/nm.4077 (2016).
17 Wu, S., Lim, K. C., Huang, J., Saidi, R. F. & Sears, C. L. Bacteroides fragilis enterotoxin cleaves the zonula adherens protein, E-cadherin. Proc Natl Acad Sci USA 95, 14979-14984, doi:10.1073/pnas.95.25.14979 (1998).
18 Pierce, J. V., Fellows, J. D., Anderson, D. E. & Bernstein, H. D. A clostripain-like protease plays a major role in generating the secretome of enterotoxigenic Bacteroides fragilis. Mol Microbiol 115, 290-304, doi:10.1111/mmi.14616 (2021).
19 Lauber, F., Cornelis, G. R. & Renzi, F. Identification of a New Lipoprotein Export Signal in Gram-Negative Bacteria. mBio 7, doi:10.1128/mBio.01232-16 (2016).
20 Herrou, J., Choi, V. M., Bubeck Wardenburg, J. & Crosson, S. Activation Mechanism of the Bacteroides fragilis Cysteine Peptidase, Fragipain. Biochemistry 55, 4077-4084, doi:10.1021/acs.biochem.6b00546 (2016).
21 Sineva, E., Savkina, M. & Ades, S. E. Themes and variations in gene regulation by extracytoplasmic function (ECF) sigma factors. Curr Opin Microbiol 36, 128-137, doi:10.1016/j.mib.2017.05.004 (2017).
22 Ndamukong, I. C., Gee, J. & Smith, C. J. The extracytoplasmic function sigma factor EcfO protects Bacteroides fragilis against oxidative stress. J Bacteriol 195, 145-155, doi:10.1128/JB.01491-12 (2013).
23 Giddings, K. S., Zhao, J., Sims, P. J. & Tweten, R. K. Human CD59 is a receptor for the cholesterol-dependent cytolysin intermedilysin. Nat Struct Mol Biol 11, 1173-1178, doi:10.1038/nsmb862 (2004).
24 Di Lorenzo, F. et al. Pairing Bacteroides vulgatus LPS Structure with Its Immunomodulatory Effects on Human Cellular Models. ACS Cent Sci 6, 1602-1616, doi:10.1021/acscentsci.0c00791 (2020).
25 Krogh, T. J., Agergaard, C. N., Moller-Jensen, J. & Justesen, U. S. Draft Genome Sequence of Parabacteroides goldsteinii with Putative Novel Metallo-beta-Lactamases Isolated from a Blood Culture from a Human Patient. Genome Announc 3, doi:10.1128/genomeA.00937-15 (2015).
26 Liu, W. et al. Unique Features of Ethnic Mongolian Gut Microbiome revealed by metagenomic analysis. Scientific Rep6, 34826, doi:10.1038/srep34826 (2016).
27 Groussin, M. et al. Elevated rates of horizontal gene transfer in the industrialized human microbiome. Cell 184, 2053-2067 e2018, doi:10.1016/j.cell.2021.02.052 (2021).
28 Segata, N. et al. Metagenomic microbial community profiling using unique clade-specific marker genes. Nature methods 9, 811-814, doi:10.1038/nmeth.2066 (2012).
29 Gordon, V. M. & Leppla, S. H. Proteolytic activation of bacterial toxins: role of bacterial and host cell proteases. Infect Immun 62, 333-340, doi:10.1128/iai.62.2.333-340.1994 (1994).
30 McCaughey, L. C. et al. Lectin-like bacteriocins from Pseudomonas spp. utilise D-rhamnose containing lipopolysaccharide as a cellular receptor. PLoS Path 10, e1003898, doi:10.1371/journal.ppat.1003898 (2014).
31 Ghequire, M. G. K., Swings, T., Michiels, J., Buchanan, S. K. & De Mot, R. Hitting with a BAM: Selective Killing by Lectin-Like Bacteriocins. mBio 9, doi:10.1128/mBio.02138-17 (2018).
32 Teanpaisan, R., Narawatthana, S. & Utarabhand, P. The gene coding for nigrescin produced by Prevotella nigrescens ATCC 25261. Lett Appl Microbiol 49, 293-298, doi:10.1111/j.1472-765X.2009.02657.x (2009).
33 Kaewsrichan, J., Douglas, C. W., Nissen-Meyer, J., Fimland, G. & Teanpaisan, R. Characterization of a bacteriocin produced by Prevotella nigrescens ATCC 25261. Lett Appl Microbiol 39, 451-458, doi:10.1111/j.1472-765X.2004.01608.x (2004).
34 Pantosti, A., Tzianabos, A. O., Onderdonk, A. B. & Kasper, D. L. Immunochemical characterization of two surface polysaccharides of Bacteroides fragilis. Infect Immun 59, 2075-2082 (1991).
35 Garcia-Bayona, L. & Comstock, L. E. Streamlined Genetic Manipulation of Diverse Bacteroides and Parabacteroides Isolates from the Human Gut Microbiota. mBio 10, doi:10.1128/mBio.01762-19 (2019).
36 Tang, Y. P. & Malamy, M. H. Isolation of Bacteroides fragilis mutants with in vivo growth defects by using Tn4400′, a modified Tn4400 transposition system, and a new screening method. Infect Immun 68, 415-419 (2000).
37 Wu, M. et al. Genetic determinants of in vivo fitness and diet responsiveness in multiple human gut Bacteroides. Science 350, aac5992, doi:10.1126/science.aac5992 (2015).
38 Smith, C. J., Parker, A. & Rogers, M. B. Plasmid transformation of Bacteroides spp. by electroporation. Plasmid 24, 100-109 (1990).
39 Chatzidaki-Livanis, M., Geva-Zatorsky, N. & Comstock, L. E. Bacteroides fragilis type VI secretion systems use novel effector and immunity proteins to antagonize human gut Bacteroidales species. Proc Natl Acad Sci USA 113, 3627-3632, doi:10.1073/pnas.1522510113 (2016).
40 Zitomersky, N. L., Coyne, M. J. & Comstock, L. E. Longitudinal analysis of the prevalence, maintenance, and IgA response to species of the order Bacteroidales in the human gut. Infect Immun 79, 2012-2020, doi:IAI.01348-10 [pii]10.1128/IAI.01348-10 (2011).
41 Shepard, L. A. et al. Identification of a membrane-spanning domain of the thiol-activated pore-forming toxin Clostridium perfringens perfringolysin O: an alpha-helical to beta-sheet transition identified by fluorescence spectroscopy. Biochemistry 37, 14563-14574, doi:10.1021/bi981452f (1998).
42 Ramachandran, R., Tweten, R. K. & Johnson, A. E. Membrane-dependent conformational changes initiate cholesterol-dependent cytolysin oligomerization and intersubunit beta-strand alignment. Nat Struct Mol Biol 11, 697-705, doi:10.1038/nsmb793 (2004).
43 Davis, M. R., Jr. & Goldberg, J. B. Purification and visualization of lipopolysaccharide from Gram-negative bacteria by hot aqueous-phenol extraction. JoVE, doi:10.3791/3916 (2012).
44 Kolmogorov, M., Yuan, J., Lin, Y. & Pevzner, P. A. Assembly of long, error-prone reads using repeat graphs. Nat. Biotechnol. 37, 540-546, doi:10.1038/s41587-019-0072-8 (2019).
45 Garcia-Bayona, L., Coyne, M. J. & Comstock, L. E. Mobile Type VI secretion system loci of the gut Bacteroidales display extensive intra-ecosystem transfer, multi-species spread and geographical clustering. PLoS Genetics 17, e1009541, doi:10.1371/journal.pgen.1009541 (2021).
46 Langmead, B. & Salzberg, S. L. Fast gapped-read alignment with Bowtie 2. Nat. Methods 9, 357-359, doi:10.1038/nmeth.1923 (2012).
47 Danecek, P. et al. Twelve years of SAMtools and BCFtools. Gigascience 10, doi:10.1093/gigascience/giab008 (2021).
48 Quinlan, A. R. & Hall, I. M. BEDTools: a flexible suite of utilities for comparing genomic features. Bioinformatics 26, 841-842, doi:10.1093/bioinformatics/btq033 (2010).
49 Love, M. I., Huber, W. & Anders, S. Moderated estimation of fold change and dispersion for RNA-seq data with DESeq2. Genome Biology 15, 550, doi:10.1186/s13059-014-0550-8 (2014).
50 Robinson, M. D., McCarthy, D. J. & Smyth, G. K. edgeR: a Bioconductor package for differential expression analysis of digital gene expression data. Bioinformatics 26, 139-140, doi:10.1093/bioinformatics/btp616 (2010).
51 Hernandez-Salmeron, J. E. & Moreno-Hagelsieb, G. Progress in quickly finding orthologs as reciprocal best hits: comparing blast, last, diamond and MMseqs2. BMC Genomics 21, 741, doi:10.1186/s12864-020-07132-6 (2020).
52. Shkoporov, A. N. et al. Analysis of a novel 8.9 kb cryptic plasmid from Bacteroides uniformis, its long-term stability and spread within human microbiota. Plasmid 69, 146-159, doi:10.1016/j.plasmid.2012.11.002 (2013).
53. Zitomersky N L, Coyne M J, Comstock L E. Longitudinal analysis of the prevalence, maintenance, and IgA response to species of the order Bacteroidales in the human gut. Infect Immun. 2011 May; 79(5):2012-20. doi: 10.1128/IAI.01348-10. Epub 2011 Mar. 14. PMID:21402766; PMCID: PMC3088145.
54. Laclare McEneany, V., Coyne, M. J., Chatzidaki-Livanis, M. & Comstock, L. E. Acquisition of MACPF domain-encoding genes is the main contributor to LPS glycan diversity in gut Bacteroides species. ISME J 12, 2919-2928, doi:10.1038/s41396-018-0244-4 (2018).
55. Roelofs, K. G., Coyne, M. J., Gentyala, R. R., Chatzidaki-Livanis, M. & Comstock, L. E. Bacteroidales Secreted Antimicrobial Proteins Target Surface Molecules Necessary for Gut Colonization and Mediate Competition In Vivo. mBio 7, doi:10.1128/mBio.01055-16 (2016).

1. A bacterial cell or population of cells that comprise at least one of:
i) a heterologous nucleic acid encoding a ECF-type sigma factor, or a functional fragment thereof;
ii) deletion of a functional ECF-type-associated anti-sigma factor; or
iii) a heterologous nucleic acid encoding a ECF-type sigma factor-induced gene and/or an O-antigen inducing gene.

2. The cell(s) of claim 1, wherein the ECF-type-associated anti-sigma factor is in the same operon as the ECF-type sigma factor.

3. The cell(s) of claim 1 or 2, wherein the anti-sigma factor comprises a beta-barrel outer membrane structure at the C-terminus, a periplasmic region, and an inner membrane domain near the N-terminus.

4. The cell(s) of any one of claims 1-3, wherein the cell comprises a deletion of a functional ECF-type-associated anti-sigma factor and deletion of a functional endogenous ECF-type sigma factor.

5. The cell(s) of any one of claims 1-4, wherein the cell(s) are further classified as Gram negative bacterial cells.

6. The cell(s) of any one of claims 1-5, wherein the bacterial cell is a Bacteroidales order.

7. The cell(s) of claim 6, wherein the bacterial cell is of the genus Prevotella, Phocaeicola, Alistipes, Parabacteroides, Odoribacter, or Bacteroides.

8. The cell(s) of any one of claims 1-7, wherein the ECF-type sigma factor, functional fragment thereof, associated anti-sigma factor, or sigma factor-induced gene is from P. vulgatus or P. dorei.

9. The cell(s) of any one of claims 1-8, wherein the cell(s) are from Phocaeicola vulgatus, and comprise a deletion of a functional ECF-type-associated anti-sigma factor of SEQ ID NO:2.

10. The cell(s) of any one of claims 1-9, wherein the heterologous nucleic acid encodes for a sigma factor comprising the amino acid sequence of one of SEQ ID NO:1 or 95-99 or an amino acid sequence with at least 70% sequence identity or similarity to one of SEQ ID NO:1 or 95-99.

11. The cell(s) of claim 10, wherein the nucleic acid encodes for a protein comprising one or more amino acid substitutions relative to one of SEQ ID NO:1 or 95-99.

12. The cell(s) of claim 11, wherein the one or more amino acid substitutions are conservative substitutions.

13. The cell(s) of any one of claims 1-12, wherein the heterologous nucleic acid is on a plasmid or is inserted into the bacteria's genome.

14. The cell(s) of any one of claims 1-13, wherein the ECF-type sigma factor comprises a sigma factor in the same operon as an anti-sigma factor comprising a beta-barrel outer-membrane structure.

15. The cell(s) of claim 14, wherein the anti-sigma factor comprises an amino acid sequence of one of SEQ ID NOS:4-8.

16. The cell(s) of any one of claims 1-15, wherein the ECF-type sigma factor-induced gene comprises a gene encoding a protein with a NigD-like domain (pfam17415 or pfam12667) or ortholog.

17. The cell(s) of any one of claims 1-16, wherein the ECF-type sigma factor-induced gene comprises a gene selected from MON98_03293, MON98_09762, MON98_02415, MON98_01358, MON98_02916, MON98_01737, MON98_02416, MON98_00321, MON98_02417, MON98_01738, MON98_01576, and/or MON98_01669 and orthologs thereof.

18. The cell(s) of any one of claims 1-17, wherein the ECF-type sigma factor-induced gene encodes for a protein of one of SEQ ID NOS:9-16 or an amino acid sequence with at least 70% sequence identity or similarity to one of SEQ ID NOS:9-16.

19. The cell(s) of claim 18, wherein the nucleic acid encodes for a protein comprising one or more amino acid substitutions relative to one of SEQ ID NOS:9-16.

20. The cell(s) of claim 19, wherein the one or more amino acid substitutions are conservative substitutions.

21. The cell(s) of any one of claims 1-20, wherein the cells further comprise a heterologous nucleic acid encoding a transgene.

22. The cell(s) of claim 21, wherein the transgene comprises an enzyme, a cytotoxic protein, or a therapeutic molecule.

23. The cell(s) of any one of claims 1-22, wherein the heterologous nucleic acid comprises DNA.

24. The cell(s) of any one of claims 1-23, wherein the cells comprise a protein or fragment thereof expressed from the heterologous nucleic acid.

25. A method comprising administering BcpT or BSAP-3 to the cell(s) of any one of claims 1-24.

26. A method comprising administering BcpT or BSAP-3 to a population of bacterial cells.

27. A method for increasing the engraftment potential in a population of bacterial cells, the method comprising administering BcpT or BSAP-3 to the population of bacterial cells.

28. The method of claim 26 or 27, wherein the cells are further classified as Gram negative bacterial cells.

29. The method of any one of claims 26-28, wherein the bacterial cells are Bacteroidales order.

30. The method of any one of claims 26-29, wherein the bacterial cells are of the genus Prevotella, Phocaeicola, Alistipes, Parabacteroides, Odoribacter, or Bacteroides.

31. The method of any one of claims 25-30, wherein the BcpT or BSAP-3 is administered at a concentration of about 1-20 μg/ml.

32. A population of bacterial cells produced by the method of any one of claims 25-31.

33. A composition comprises the cell(s) of any one of claims 1-24 or 32 and a carrier.

34. The composition of claim 33, wherein the cell(s) are further defined as live cells.

35. The composition of claim 33 or 34, wherein the composition is further formulated as a tablet, capsule, or food product.

36. A method comprising administering the composition of any one of claims 33-35 to a subject in need thereof.

37. A method for increasing bacterial populations in the gut of a subject comprising administering the composition of any one of claims 33-35 to a subject in need thereof.

38. A method for increasing the engraftment of bacterial populations in the gut of a subject comprising administering the composition of any one of claims 33-35 to a subject in need thereof.

39. The method of claim 37 or 38, wherein the bacterial population comprises Bacteroidales order.

40. The method of any one of claims 36-39, wherein the subject is a human subject.
